SEQUENCE LISTING (2 sequences)

SEQ ID NO: 1 - protein, 6 aa, Artificial Sequence
  Synthetic construct
WYRGRL

SEQ ID NO: 2 - protein, 6 aa, Artificial Sequence
  Synthetic construct
YRLGRW

(19) United States
(12) Patent Application Publication
(10) Pub. No.: US 2017/0050988 A1
(43) Pub. Date: Feb. 23, 2017

(54) DOTAM DERIVATIVES FOR THERAPEUTIC USE

(71) Applicants: SANOFI, Paris (FR); EUROPEAN MOLECULAR BIOLOGY LABORATORY, Heidelberg (DE)

(72) Inventors: Hai-Yu HU, Frankfurt (DE); Marc NAZARE, Paris (FR); Ngee HAN LIM, Oxford (GB); Danping DING-PFENNIGDORFF, Paris (FR); Oliver PLETTENBURG, Paris (FR); Olaf RITZELER, Paris (FR); Hans-Paul JURETSCHKE, Paris (FR); Joachim SAAS, Paris (FR); Eckart BARTNIK, Paris (FR); Peter FLORIAN, Paris (FR); Ulrich WENDT, Paris (FR); Carsten SCHULTZ, Heidelberg (DE); Hideaki NAGASE, Oxford (GB)

(73) Assignees: SANOFI, Paris (FR); EUROPEAN MOLECULAR BIOLOGY LABORATORY, Heidelberg (DE)

(21) Appl. No.: 15/039,068

(22) PCT Filed: Nov. 25, 2014
(86) PCT No.: PCT/IB2014/066327

§ 371 Date: May 25, 2016

(30) Foreign Application Priority Data
Nov. 25, 2013 (EP) 13306609.2

(51) Int. Cl. C07F 5/00; A61K 49/04; A61K 49/14; C07K 7/06

(52) U.S. Cl. CPC C07F 5/003; C07K 7/06; A61K 49/04; A61K 49/14; A61K 38/00

(57) ABSTRACT

A compound of formula (I), a stereoisomeric form thereof, or a pharmaceutically acceptable salt thereof, wherein the substituents are as defined herein, is provided. The compounds of formula (I) are valuable pharmacologically active compounds and are suitable for the treatment of osteoarthritis. The preparation of the compounds of formula (I), their use as pharmaceuticals, and pharmaceutical compositions including them are also provided.

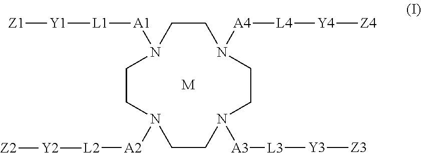

The invention relates to DOTAM derivatives for therapeutic use as drug delivery agents, diagnostic and contrast agents. More specifically the invention relates to drug delivery agents, diagnostic and contrast agents comprising targeting moieties which can bind to cartilage and allow the localized diagnosis and monitoring of degenerative joint disorders or inflammatory processes such as osteoarthritis or rheumatism.

The present invention relates to 1,4,7,10-Tetraazacyclododecane-1,4,7,10-tetraacetic acid amide derivatives of the formula (I),

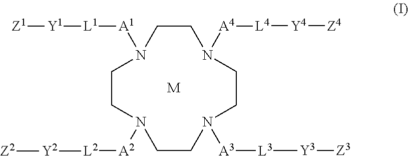

wherein Z¹⁻⁴, Y¹⁻⁴, L¹⁻⁴, A¹⁻⁴, and M is absent or present, and have the meanings indicated below for the formula (Ia) and formula (Ib).

According to a first aspect, the present invention relates to compounds of formula (Ia) as diagnostic and contrast agents. According to a second aspect, the present invention related to compounds of formula (Ib) as drug delivery agents.

The invention furthermore relates to processes for the preparation of the compounds of the formula (Ia) and (Ia), their use as pharmaceuticals, and pharmaceutical compositions comprising them.

Osteoarthritis (OA) is one of the most common degenerative joint diseases and leads in an advanced stage to a loss of joint function. During the progression of the disease articular cartilage is continuously being destructed down to the underlying bone tissue, which makes a joint replacement surgery in affected patients necessary. In addition to the destruction of the cartilage, pathological changes in subchondral bone, the synovial membrane and the ligaments can also be observed. The disease is temporarily accompanied by inflammatory processes like in rheumatoid arthritis, but differs from it. The exact causes of the disease are still unknown; however, several factors come into question, such as metabolic changes, mechanical stress, genetic disorders or joint injuries. Regardless of the original trigger, the degradation of articular cartilage occurs as a common pathological feature of OA. A key feature of the pathological condition of OA is the proteolytic cleavage of extracellular matrix components e.g. collagens and proteoglycans. Simultaneously a number of other processes occur such as chondrocyte phenotype loss, cell death and attempted anabolic repair mechanisms which fail to regenerate functional cartilage tissue. The precise molecular mechanisms underlying these processes are still poorly understood.

The healthy functioning of the adult cartilage is created by its unique biomechanical properties, providing both the resistance against high pressure as well as the necessary elasticity of the tissue. The decisive factor is the special organization of the cartilage tissue. Unlike most other tissues, the cartilage cells are not in direct contact but are embedded separately from each other in an extracellular matrix (ECM). The macromolecules of this ECM guarantee the viability of the articular cartilage and joints. The basic structure of the ECM consists of a network that is formed by fibrils of collagen types II, IX and XI. Proteoglycans, mainly aggrecan, are embedded in the ECM producing an extremely high osmotic water binding capacity. The water pressure generated in connection with the properties of the collagen backbone guarantee the specific properties of the cartilage. The early or pre-symptomatic articular pathology is characterized by the degradation and loss of glycosaminoglycan (GAG) chains which are associated with the collagen—mainly collagen II-fibers. It is believed that these GAG chains protect the collagen fibers from excessive proteolytic cleavage and that in consequence to the loss of GAG the collagen fibers are accessible to proteolytic enzymes and the degradative cleavage/processing causes then a loosening of the collagen network and decreased collagen II content. These subtle biochemical changes translate into the manifestation of soft focal lesions, where collagen II fibers are exposed and the surrounding tissue is accessible to further proteolytic cleavage and degradation.

A main feature of the pathogenesis of OA is the loss of the ECM of articular cartilage tissue. Ultimately, joint function is restricted or lost. Besides structural and functional degeneration, OA is characterized by pain.

Diagnosis and monitoring of the early stages of the disease is difficult due to the lack of specific biomarkers and due to the fact that clinical symptoms of osteoarthritis like pain and stiffness of the joint often correspond to the later, progressed stages of the disease which will then eventually be diagnosed by radiographic or Magnetic Resonance Imaging (MRI) techniques.

Treatment approaches for osteoarthritis entail cartilage matrix protecting or restoring principles, targeting bone remodeling and improvement of symptoms. Since breakdown of the ECM (Aggrecan and Collagen) belong to the earliest histopathological lesions in OA, the development of protease inhibitors against MMPs, Adamts and Cathepsins is of therapeutic relevance.

Specific Matrix metalloproteinase inhibitors, in particular MMP13-specific inhibitors were discovered and tested in preclinical models of osteoarthritis (Johnson A R et al. J Biol Chem. 2007 282(38):27781-91. Discovery and characterization of a novel inhibitor of matrix metalloprotease-13 that reduces cartilage damage in vivo without joint fibroplasia side effects; Baragi, V. M. et al. A new class of potent matrix metalloproteinase 13 inhibitors for potential treatment of osteoarthritis: Evidence of histologic and clinical efficacy without musculoskeletal toxicity in rat models. Arthritis Rheum. 60, 2008-2018 (2009).

Aggrecanase inhibitors, inhibiting Adamts-4 and -5, were developed and clinical trials with Aggrecanase inhibitor AGG-523 triggered (US National Library of Medicine. ClinicalTrials.gov [online], http://www.clinicaltrials.gov/ct2/how/NCT00427687 (2007)).

Cathepsin K inhibitors show DMOAD potential in several experimental models of osteoarthritis (Inhibition of cathepsin K reduces cartilage degeneration in the anterior cruciate ligament transection rabbit and murine models of osteoarthritis Hayami T et al. Bone. 2012 June; 50(6):1250-9; Cathepsin K inhibition reduces CTXII levels and joint pain in the guinea pig model of spontaneous osteoarthritis. McDougall J J, et al. Osteoarthritis Cartilage. 2010 (10):1355-7. Other cathepsins including cathepsin B and D came into focus due to their ability to degrade proteoglycan at neutral or acidic pH, respectively. Particularly, in vitro proteoglycan degradation cartilage explants under acidic conditions could be inhibited with pepstatin, a highly active inhibitor of Cathepsin D (Dingle, J. T., et al. Inhibition by Pepstatin of Human Cartilage Degradation Biochem. J. (1972) 127, 443 444).

Pro-anabolic growth factor BMP7 has been shown to inhibit progression of OA and has the capacity to repair cartilage in vivo. Phase I clinical studies demonstrated safety of BMP7 in patients with human OA (Hunter, D. J. et al. Phase 1 safety and tolerability study of BMP-7 in symptomatic knee osteoarthritis. BMC Musculoskelet. Disord. 11, 232 (2010).) FGF18, an anabolic growth factor, with DMOAD activity in the rat, is undergoing clinical trials (McPherson, R., Flechsenhar, K., Hellot, S. & Eckstein, F. A randomized, double blind, placebo-controlled, multicenter study of rhFGF18 administered intraarticularly using single or multiple ascending doses in patients with primary knee osteoarthritis (OA), not expected to require knee surgery within 1 year. Osteoarthritis Cartilage 19 (Suppl. 1), 35-36 (2011).

Several therapeutic approaches targeting bone and cartilage remodeling are also in the clinical test phase at different stages: Strontium Ranelate (Reginster, J. Y. et al. Efficacy and saftey of strontium ranelate in the treatment of knee osteoarthritis: results of a double-blind, randomised placebo-controlled trial. Ann. Rheum. Dis. 72, 179-186 (2013); Calcitonin (Karsdal, M. A. et al. Oral calcitonin demonstrated symptom-modifying efficacy and increased cartilage volume: results from a 2-year phase 3 trial in patients with osteoarthritis of the knee. Osteoarthritis Cartilage 19 (Suppl. 1), 35 (2011)); iNOS-inhibiton has the potential to control symptoms of OA and in addition to effect structural progression of OA (Hellio le Graverand, M. P. et al. A 2-year randomized, double-blind, placebo controlled, multicenter study of oral selective iNOS inhibitor, cindunistat (SD-6010), in patients with symptomatic osteoarthritis of the knee. Ann. Rheum. Dis. 72, 187-195 (2012).)

Depending on the characteristics of the injected drug or therapeutic principle residence times below 1-2 hours are observed. For example, NSAIDs (non steroidal anti inflammatory drugs) or dissolved corticosteroids are typically eliminated within 1-2 hours and have therefore a much too short residence time for a sustained long-term treatment via intraarticular administration.

Currently available approaches to extend/prolong the residence time of a drug rely on formulations that form a passive depot in the joint space and delaying the release the active ingredient in the synovial fluid. Such formulations are for example liposomes, hydrogels (hyaluronic acid and modified derivatives thereof), crystalline suspensions of poorly soluble drugs, microparticles and nanoparticles (e.g. poly(lactic-co-glycolic acid)/PLGA, polylactic acid/PLLA). Many of these approaches suffer from a poor control of the drug release rates of the encapsulated active ingredient showing a dependency of the release to the force load of the joint as well as insufficient prolongation of the residence time. Moreover, in particular the particle based approaches, like suspensions and microparticles can induce severe side-effects like painful inflammation (synovitis) with joint swelling, cellular infiltration and even to minor levels proteoglycan loss. Targeted drug delivery aims to increase the concentration and residence time of the drug in the tissue of interest while reducing the relative concentration in the other tissues. With a selective and differential distribution, the therapeutic index of the drug as a measure of its pharmacological response and safety is improved minimizing the overall toxicity while enhancing the therapeutic efficacy.

In contrast to the passive targeting of the articular cartilage by localized administration and deposition in the joint space, an active targeting approach aims to retain the active ingredient in joint tissue by having a distinct affinity to the cartilage tissue or tissue components thereof such as collagen or GAG.

Compared with polymer counterparts, small molecule-based carriers offer unique advantages, including well-defined molecular structure, definite molecular weight, and high purity without batch to batch variations; however, according to our knowledge, small molecule-based carriers for increasing retention in the joint have not been reported.

The local application of a diagnostic agent via intrarticular administration offers several advantages over the systemic administration and is suitable to minimize or avoid adverse side effects to the cardiovascular, renal, gastrointestinal or central-nervous system. In principle high levels at the target tissue (100% bioavailability) can be achieved which might not be reached at all by systemic application, either because of poor oral bioavailability, first pass metabolism or specific distribution characteristics and with the advantage of a significant reduced dose.

Moreover, for the vast majority of OA patients, only one joint such as knee, hand or feet joint which are accessible by intraarticular administration is affected by the disease.

The articular cartilage in the joint space as primary site of the disease is however a remote location which is due to its avascularity only connected with the systemic circulation via the synovial fluid. However, the joint space is a heavily rinsed compartment and dissolved small molecules which are present in the joint after local administration via intrarticular administration are rapidly cleared within hours by convective transport and lymphatic uptake.

It is therefore apparent that there exists a medical need for a localized, cartilage tissue selective monitoring of cartilage defects allowing diagnosis of early or pre-symptomatic articular pathology of osteoarthritis and also for an active targeting cartilage selective drug delivery to cartilage defects allowing a highly localized and sustained treatment of early or pre-symptomatic articular pathology of OA.

In addition it is also advantageous to monitor the fate of a drug carrier as of its administration instantaneously, and the fact that the drug is released such monitoring is desired to determine in advance if, when and where the drug release takes place. Such a theranostic agent can be useful for the in vivo visualization of the drug delivery of the release as well as the therapy monitoring (theranosis). In that way, different applications like drug delivery, drug release, monitoring of therapy and interventional imaging can be combined. For example the localization of a therapeutic agent can be followed by incorporation of a MRI contrast enhancing Gadolinium(III) containing agent or an near infrared label. (E. Terreno et al. J. Control. Release 161 (2012) 328-337; B. D. Smith et al. Bioconjugate Chem. 23 (2012) 1989-2006; N. Zhang et al. Biomaterials 33 (2012) 5363-5375).

Magnetic Resonance Imaging (MRI) is a major advance in the non-destructive imaging of human soft tissues like e.g. cartilage. However despite high contrast and resolution as well as the capability for multiplanar visualisation MRI often depicts only moderate to severe disease related changes of the cartilage tissue. To enhance the contrast and improve the visibility between different regions of the tissue, contrast agents such as gadopentetate (Magnevist), gadoterate (Dotarem), gadodiamide (Omniscan), gadobenate (MultiHance), gadoteridol (ProHance), gadoversetamide (OptiMARK), gadoxetate (Primovist) and gadobutrol (Gadovist) are currently employed. These low molecular weight gadolinium(III) complexes are paramagnetic complexes which shorten the longitudinal T1 relaxation time and the transversal T2 relaxation time of the protons located nearby. Gd(III) derived T1 shortening increases the signal intensity in T1-weighted imaging and causes a brightening of these images.

Other lanthanide(III) ion complexes suitable for improving the sensitivity and contrast of MRI are for example Europium(III), or Dysprosium(III).

In general, the currently available MRI CA are not selectively targeting a certain tissue. However, the possible impact and utility of a tissue selective enrichment of a CA can be demonstrated by the widespread application of the delayed gadolinium MRI of cartilage (dGMERIC) technique for the early diagnosis of OA. At present this is the only clinically accepted method suitable to assess early cartilage quality changes and has proven to be predictive for future joint degeneration. As already mentioned early OA is characterized by a decreased glycosaminoglycan (GAG) content. GAGs (e.g. chontroitinsulfate and keratinsulfate are highly negatively charged extensive polysaccharides linked to aggrecan protein backbone.

dGMERIC exploits the fact that the negatively charged gadopentetate Gd(DTPA)²⁻ is enriched/accumulating in cartilage with decreased GAG content due to the reduced electrical negative repulsion relative to normal cartilage. This results in a shorter T1 relaxation time or higher MRI signal intensity at regions of low GAG content which corresponds to regions of early biochemical changes and tissue lesions. Thus, the GAG content is directly proportional to the T1 relaxation time, which is in this setting T1 is also termed dGMERIC index. The dGMERIC index is a recognized imaging biomarker predictive for the early cartilage changes in OA. [A. Bashir et al. Magn Reson Med. 1999; 41,857; F. Eckstein et al. Osteoarthritis and Cartilage, 2006, 14, 974; T. E. McAlindon et al. Osteoarthritis and Cartilage, 2011, 19, 399.]

Another major drawback of available CA is the limited retention time and the difficulty to reach appropriate concentrations of the CA in the target tissue i.e. cartilage. Many CA tend to be cleared rapidly from the body requiring limiting the imaging procedure to a very short time after administration of such an agent. This limits the time and number of MRI images which can be recorded under similar conditions and are necessary for an unambiguous diagnosis. A highly desirable property of MRI CA is therefore to reach high and persistent concentrations at the site of investigation.

Tissue selective CA enhance the contrast in the area of diagnostic relevance and remain longer in the diseased area, ensuring sufficient time for the completion of the imaging procedure. In addition, such tissue selective agents are able to reach a higher local concentration of the CA in a given setting and thus reducing the overall amount required to provide a suitable enhanced signal.

Assessing the activity of localized disease relevant proteins in a certain tissue or cell compartment can be an important diagnostic measure for the determination of the disease state or monitoring the success of therapeutic measures.

This is in particularly important as many proteins and enzymes are regulated by a complex array of post-translational mechanisms and thus alterations in the expression levels/abundance may not correlate with the change in activity. For this purpose activity-based probes (ABPs) can be used to covalently label target proteins in their native environment and thereby providing a powerful tool assessing the disease relevant, aberrant enzyme activity at the physiological relevant site of action. To date such ABP have been developed for many enzyme classes like for example serine hydrolases (phospholipases), metalloproteinases, cysteine, serine, threonine and aspartic proteases, oxidoreducatases, phosphatases and kinases.

In principle an ABP consists of three elements: a reactive functional group (warhead), a linker which separates the reactive functional group from the tag and enables visualization and detection of the covalent protein-probe complex. Additionally, some probes may contain an additional binding group e.g. a group which resembles the native substrate or an inhibitor as recognition element which improves the specificity of the probe and enhances selectivity versus a related subset of enzymes by addressing specific residues. The reactive functional group usually consists of an electrophile which reacts with a catalytic residue of the active site of the enzyme and forms a covalent bond with the active site nucleophile. The tag of such an ABP may consist of a gamma-ray emitter for radionuclide szintigraphic tomography (e.g. ^{99m}Tc, ¹⁷⁷Lu), a positron emitter for positron-emission tomography (PET) (e.g. ⁶⁸Ga, ⁶⁴Cu), magnetic resonance imaging (MRI) (e.g. Gd, Yb, Eu, Nd) or a fluorecent label (e.g. optical imaging moieties like cyanine dyes)

In particular useful for in-vivo optical imaging is a further development of the ABPs, the fluorescently quenched ABPs (qABPs). These probes carry a reactive functional group with a leaving group which is detached/released upon the covalent reaction with the enzymes active site. By attaching a fluorescent quencher to the leaving group a dark quenched fluorogenic probe is obtained, which only emits a fluorescent signal after cleavage and covalent modification of the reacting enzyme. By that way, a real time, non-invasive, high resolution imaging with a maximum signal to background ratio in cellular systems or living organisms can be obtained. Beside the qABPs which contain a reactive functional group to covalently modify the processing enzyme dark quenched fluorogenic probes variants lacking such an element but consisting of the native substrate sequence have been introduced and proved to be useful. Such dark quenched fluorogenic probes have have been successfully used to detect the disease state and to follow disease progression in an OA rat model [Kim et al. Amino Acids (2011) 41, 1113; Kim et al. Bioconjugate Chemstry (2008), 19, 1743.

More recently bimodal MRI/optical imaging CAs were introduced using dual labeled MRI and near-infrared fluorescence reporters in one imaging agent. By administering such an bimodal CA for example pre-operative MRI imaging and intraoperative fluorescence signals can be useful to guide surgery and further histological analysis. In contrast to the administration of separate labeling agents often having distinct pharmacokinetic properties and imaging time windows a bimodal CA allows the investigation in the same time corridor/interval synchronously.

Optical imaging and MRI are complementary analytical techniques which can be used synchronously to obtain much more complete information regarding tissue and organs like e.g. cartilage because no single imaging modality can provide overall structural and functional information. MRI is characterized by imaging of tissues at unlimited depth high spatial and temporal resolution but is hampered by low sensitivity which is still too-low for cellular imaging. In contrast fluorescent signals for optical imaging has high sensitivity but suffers from a limited tissue penetration and low resolution which limits the applicability for in-vivo imaging. Among the fluorescent reporters near infrared (NIR) labels for imaging are more compatible with biological applications due to the lack of native NIR fluorescence in biological systems, the low′scattering of NIR photons and the fact that NIR photons can cross significant tissue depths for non-invasive in-vivo imaging.

Moreover intracellular imaging which to date might not be accomplished with the currently available MRI CA. However this is common practice with cell permeable optical imaging agents. Thus, a bimodal cell permeable CA would combine the advantages of both techniques and allow diagnostic imaging at a higher resolution and sensitivity.

A bimodal MRI/optical imaging agent has the further advantage of having identical pharmacokinetics and biodistribution, tissue distribution residence time to simultaneously exploit MRI and NIR optical imaging techniques and enable a more accurate interpretation of in vivo molecular imaging experiments as well as intraoperative guidance for accurate surgical removal of tissues and tissue margins. Providing an integrated approach for a combined preoperative topographical information through MRI and intraoperative optical imaging during surgery of the diseased joint.

J. Hubbell et al. reported functionalized polymer-based nanoparticles with a specific peptide sequence WYRGRL (SEQ ID NO:1) [TrpTyrArgGlyArgLeu] display cartilage binding properties through collagen II binding. As comparison, the non-binding scrambled oligopeptide sequence YRLGRW (SEQ ID NO:2) was used as a control. (J. Hubbell et al, Nature materials (2008), 7, 248).

The highly fixed negative charge density of the sulfated and carboxylated GAGs creates a highly hydrophilic environment and bind large amount of interstitial water. In that way the reversible elasticity of the tissue resisting compression and distributing load is maintained.

According to the present invention, an agent carrying multiple positive charges in this environment is able to exhibit a prolonged residence time and achieve a higher local concentration due to the electrostatic attraction with the negatively charged GAGs.

Therefore another aspect or the present invention is the combination of the cartilage retaining properties of the WYRGRL peptide sequence with the characteristics of (poly) cationic moieties that are positively charged at physiological relevant pHs (typically in the range 5-8) and are able to interact with the highly negatively charged GAGs. The cationic moieties are preferably introduced by substituted or unsubstituted amines, guanidines and amidines.

The affinity, biodistribution and residence of a targeting moiety e.g. peptides is influenced by the attached reporter group or the attached drug or prodrug. In certain cases the reporter group, the drug or prodrug might negatively impact these properties compared to the unlabelled or non-conjugated targeting moiety. This applies in principle all the more for multimodal contrast agents. By enhancing the ratio between the targeting moiety and the diagnostic label of the contrast agent (CA), or between the targeting moiety and the conjugated drug or prodrug, via multimerisation of the targeting moiety the negative influence of the diagnostic label or of the drug or prodrug, might be reduced. Moreover, an increased tissue selectivity, improved biodistribution and residence time versus the monovalent CA might be obtained. Factors like a higher statistical frequency and probability for a ligand protein interaction, superadditivity phenomena and shielding effects of the diagnostic reporter may contribute to such an improvement.

This invention describes the synthesis and application of a cartilage targeting/collagen type II targeting peptide sequence which are optionally multivalently attached to corresponding template 1,4,7,10-tetraazacyclododecane-1,4,7,10-tetraacetic acid (DOTA) and conjugated to a Magnetic Resonance Imaging (MRI) reporter, optical fluorescent reporter or both.

According to another aspect, the invention describes the synthesis and application of a cartilage targeting/collagen type II targeting peptide sequence which are optionally multivalently attached to corresponding template 1,4,7,10-tetraazacyclododecane-1,4,7,10-tetraacetic acid (DOTA) and conjugated to a therapeutically active drug and optionally a Magnetic Resonance Imaging (MRI) reporter.

The multivalently decorated DOTA derivative serves as a defined, peptide presenting, monomeric unit which can actively target and bind to cartilage and prolong the local residence time in the joint. These characteristics may be additionally supported by introducing further (poly) cationic moieties. Thus, a rapid excretion is prevented via the synovial fluid. The compounds of this invention can be therefore used for the localized administration of a therapeutic agent or a diagnostic agent selectively binding to cartilage for the treatment of osteoarthritis or rheumatoid arthritis and associated complications. The compounds described are designed to actively target and adhere to the cartilage tissue thus preventing significant systemic plasma concentrations and exposure of the whole body of these diagnostic agent and therapeutic agent, and thus serve to prevent adverse side effects.

Thus, a subject of the present invention is the compounds of the formula (Ia), in any of their stereoisomeric forms or a mixture of stereoisomeric forms in any ratio, and the pharmaceutically acceptable salts thereof,

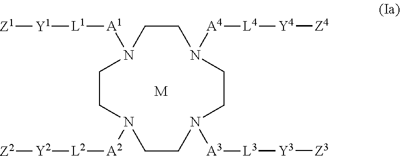

wherein

M is absent or present and a positively charged metal ion out of the group Gd, Yb, Mn, Cr, Cu, Fe, Pr, Nd, Sm, Tb, Yb, Dy, Ho, Er, Eu, Ga, ⁶⁸Ga, ⁶⁴Cu, ^{99m}Tc, ¹⁷⁷Lu, ⁶⁷Ga, ¹¹¹In, ⁹⁹Mo;

A1, A2, A3 and A4 are independently of one another, identical or different, and are independently of one another selected from a bond, —(C₀-C₄)-alkyl-C(O)—N(R1)-, —(C₀-C₄)-alkyl-P(O)ₙ—N(R1)-, —(C₀-C₄)-alkyl-S(O)ₙ—N(R1)-, —(C₀-C₄)-alkyl-N(R2)-C(O)—N(R1)- and —(C₀-C₄)-alkyl-N(R1)-C(O)—;

n is selected from 1 and 2;

L1, L2, L3 and L4 are independently of one another, identical or different, and are independently of one another selected from a bond, (C₁-C₁₈)-alkyl, —(CH₂)ₘ[—O—(CH₂)ₚ]_{q}—

m, q and p are independently of one another identical or different and are the integers zero, 1, 2, 3, 4, 5, 6, 7, 8, 9 or 10;

Y1, Y2, Y3 and Y4 are independently of one another, identical or different, and are independently of one another selected from a bond, —(C₀-C₄)-alkyl-N(R1)-, —(C₀-C₄)-alkyl-C(O)—N(R1)-, —N(R1)-C(O)—(C₀-C₆)-alkyl-, —(C₀-C₄)-alkyl-S(O)ₙ—N(R1)-, —(C₀-C₄)-alkyl-N(R2)-C(O)—N(R1)-, —(C₀-C₄)-alkyl-N(R1)-C(O)—,

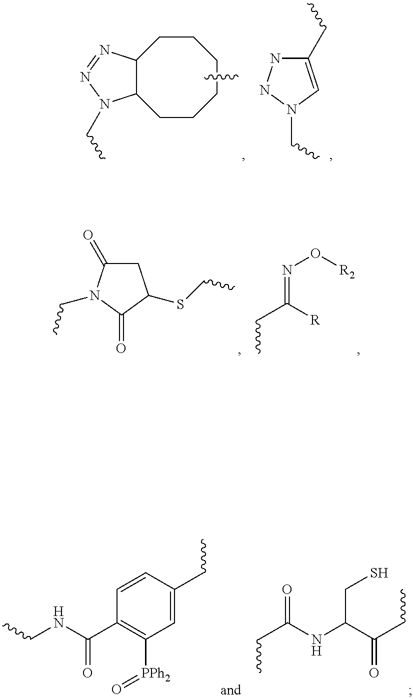

R1 and R2 are independently of one another selected from the series consisting of hydrogen, (C₁-C₄)-alkyl, (C₃-C₇)-cycloalkyl and —(C₁-C₄)-alkyl-(C₃-C₇)-cycloalkyl;

Z1, Z2, Z3 and Z4 are independently of one another, identical or different, and are independently of one another selected from:
a hydrogen atom;
a polypeptide comprising an amino acid sequence of WYRGRL-(SEQ ID NO:1) [TrpTyrArgGlyArgLeu] SEQ ID NO:I, or a conservative substitution or a repetitive sequence thereof, wherein the polypeptide specifically binds to cartilage tissue, the N-terminal moiety of the polypeptide being acetylated;
a fluorophore suitable for optical imaging;
an activity based probe (ABP) suitable to monitor the aberrant expression or activity of proteins involved in the initiation and progression of OA;
with the proviso that
at least one of Z1, Z2, Z3 and Z4 represent a polypeptide comprising an amino acid sequence of WYRGRL-(SEQ ID NO:1) [TrpTyrArgGlyArgLeu] SEQ ID NO:I, or a conservative substitution or a repetitive sequence thereof, wherein the polypeptide specifically binds to cartilage tissue, the N-terminal moiety of the polypeptide being acetylated;
at least one of Z1, Z2, Z3 and Z4 represent a fluorophore suitable for optical imaging or an activity based probe (ABP) suitable to monitor the aberrant expression or activity of proteins involved in the initiation and progression of OA;
Z1, Z2, Z3 and Z4 can not represent more than one activity based probe (ABP) suitable to monitor the aberrant expression or activity of proteins involved in the initiation and progression of OA.

Thus, a subject of the present invention is the compounds of the formula (Ib), in any of their stereoisomeric forms or a mixture of stereoisomeric forms in any ratio, and the pharmaceutically acceptable salts thereof,

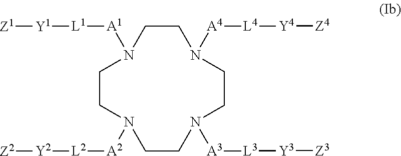

wherein

A1, A2, A3 and A4 are independently of one another, identical or different, and are independently of one another selected from a bond, —(C₀-C₄)-alkyl-C(O)—N(R1)-, —(C₀-C₄)-alkyl-P(O)ₙ—N(R1)-, —(C₀-C₄)-alkyl-S(O)ₙ—N(R1)-, —(C₀-C₄)-alkyl-N(R2)-C(O)—N(R1)- and —(C₀-C₄)-alkyl-N(R1)-C(O)—;

n is selected from 1 and 2;

L1, L2, L3 and L4 are independently of one another, identical or different, and are independently of one another selected from a bond, (C₁-C₁₈)-alkyl, —(CH₂)ₘ[—O—(CH₂)ₚ]_{q}—

m, q and p are independently of one another identical or different and are the integers zero, 1, 2, 3, 4, 5, 6, 7, 8, 9 or 10;

Y1, Y2, Y3 and Y4 are independently of one another, identical or different, and are independently of one another selected from a bond, a cleavable linker, —Y5-(Z5)ᵣ, —(C₀-C₄)-alkyl-N(R1)-, —(C₀-C₄)-alkyl-C(O)—N(R1)-, —N(R1)-C(O)—(C₀-C₆)-alkyl-, —(C₀-C₄)-alkyl-S(O)ₙ—N(R1)-, —(C₀-C₄)-alkyl-N(R2)-C(O)—N(R1)-, —(C₀-C₄)-alkyl-N(R1)-C(O)—,

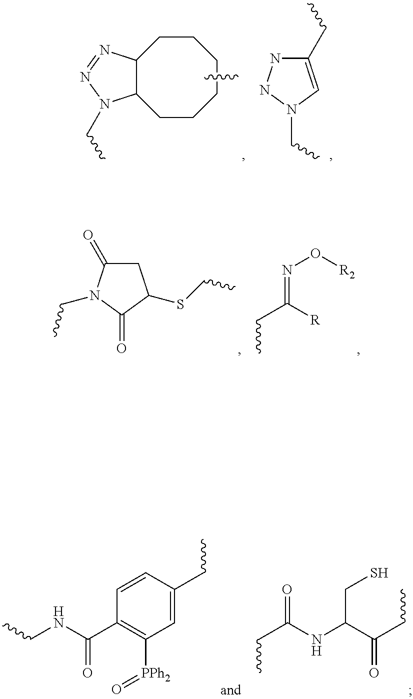

wherein is r is selected from 1 to 3, Y5 is selected from a cleavable linker, (C₀-C₄)-alkyl-N(R1), (C₀-C₄)-alkyl-C(O)—N(R1), N(R1)-C(O)—(C₀-C₆)-alkyl, (C₀-C₄)-alkyl-S(O)ₙ—N(R1), (C₀-C₄)-alkyl-N(R2)-C(O)—N(R1), (C₀-C₄)-alkyl-N(R1)-C(O), and Z5 is as defined for Z1, Z2, Z3 and Z4;

R1 and R2 are independently of one another selected from the series consisting of hydrogen, (C₁-C₄)-alkyl, (C₃-C₇)-cycloalkyl and —(C₁-C₄)-alkyl-(C₃-C₇)-cycloalkyl;

Z1, Z2, Z3 and Z4 are independently of one another, identical or different, and are independently of one another selected from:
a hydrogen atom;
a polypeptide comprising an amino acid sequence of WYRGRL-(SEQ ID NO:1) [TrpTyrArgGlyArgLeu] SEQ ID NO:I, or a conservative substitution or a repetitive sequence thereof, wherein the polypeptide specifically binds to cartilage tissue, the N-terminal moiety of the polypeptide being acetylated;
a compound useful for treatment of osteoarthritis, complications related to osteoarthritis or cartilage related disorders;
with the proviso that
at least one of Z1, Z2, Z3 and Z4 represent a polypeptide comprising an amino acid sequence of WYRGRL-(SEQ ID NO:1) [TrpTyrArgGlyArgLeu] SEQ ID NO:I, or a conservative substitution or a repetitive sequence thereof, wherein the polypeptide specifically binds to cartilage tissue, the N-terminal moiety of the polypeptide being acetylated;
at least one of Z1, Z2, Z3 and Z4 represent a compound useful for treatment of osteoarthritis, complications related to osteoarthritis or cartilage related disorders as defined above.

According to another aspect, the present invention relates to a compound of the formula (Ia), in any of their stereoisomeric forms or a mixture of stereoisomeric forms in any ratio, and the pharmaceutically acceptable salts thereof,

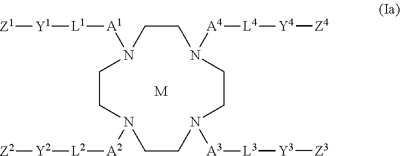

wherein

M is absent or present and a positively charged metal ion out of the group Gd, Yb, Mn, Cr, Cu, Fe, Pr, Nd, Sm, Tb, Yb, Dy, Ho, Er, Eu, Ga, ⁶⁸Ga, ⁶⁴Cu, ^{99m}TC, ¹⁷⁷Lu, ⁶⁷Ga, ¹¹¹In, ⁹⁹Mo;

A1, A2, A3 and A4 are independently of one another, identical or different, and are independently of one another selected from a bond, —(C₀-C₄)-alkyl-C(O)—N(R1)-, —(C₀-C₄)-alkyl-P(O)ₙ—N(R1)-, —(C₀-C₄)-alkyl-S(O)ₙ—N(R1)-, —(C₀-C₄)-alkyl-N(R2)-C(O)—N(R1)- and —(C₀-C₄)-alkyl-N(R1)-C(O)—;

n is selected from 1 and 2;

L1, L2, L3 and L4 are independently of one another, identical or different, and are independently of one another selected from a bond, (C₁-C₁₈)-alkyl, —(CH₂)ₘ[—O—(CH₂)ₚ]_{q}—

m, q and p are independently of one another identical or different and are the integers zero, 1, 2, 3, 4, 5, 6, 7, 8, 9 or 10;

Y1, Y2, Y3 and Y4 are independently of one another, identical or different, and are independently of one another selected from a bond, —(C₀-C₄)-alkyl-N(R1)-, —(C₀-C₄)-alkyl-C(O)—N(R1)-, —N(R1)-C(O)—(C₀-C₆)-alkyl-, —(C₀-C₄)-alkyl-S(O)ₙ—N(R1)-, —(C₀-C₄)-alkyl-N(R2)-C(O)—N(R1)-, —(C₀-C₄)-alkyl-N(R1)-C(O)—,

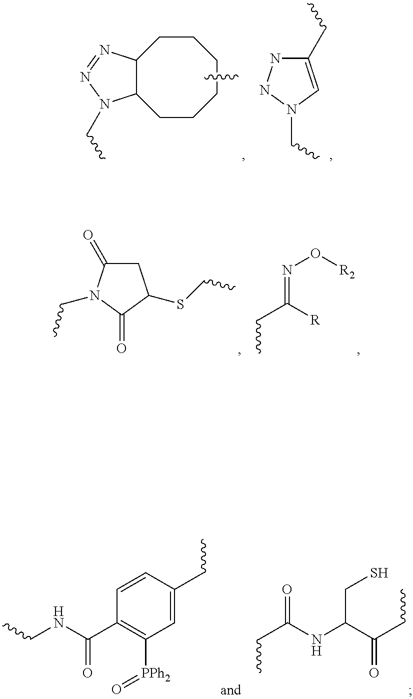

R1 and R2 are independently of one another selected from the series consisting of hydrogen, (C₁-C₄)-alkyl, (C₃-C₇)-cycloalkyl and —(C₁-C₄)-alkyl-(C₃-C₇)-cycloalkyl;

Z1, Z2, Z3 and Z4 are independently of one another, identical or different, and are independently of one another selected from:
a hydrogen atom;
a polypeptide comprising an amino acid sequence of WYRGRL-(SEQ ID NO:1) [TrpTyrArgGlyArgLeu] SEQ ID NO:I, or a conservative substitution or a repetitive sequence thereof, wherein the polypeptide specifically binds to cartilage tissue, the N-terminal moiety of the polypeptide being acetylated;
a fluorophore;
an activity based probe (ABP);
with the proviso that
at least one of Z1, Z2, Z3 and Z4 represent a polypeptide comprising an amino acid sequence of WYRGRL-(SEQ ID NO:1) [TrpTyrArgGlyArgLeu] SEQ ID NO:I, or a conservative substitution or a repetitive sequence thereof, wherein the polypeptide specifically binds to cartilage tissue, the N-terminal moiety of the polypeptide being acetylated;
at least one of Z1, Z2, Z3 and Z4 represent a fluorophore or an activity based probe (ABP);
Z1, Z2, Z3 and Z4 can not represent more than one activity based probe (ABP).

According to a further aspect, the present invention relates to a compound of the formula (Ib) as defined above, wherein the compound useful for treatment of osteoarthritis, complications related to osteoarthritis or cartilage related disorders is selected from an inhibitor of MMP13, recombinant metalloproteinase inhibitor-3, ADAMTS4/5, Cathepsin D, Cathepsin K inhibitors, inhibitors of Cathepsin S or B, serine protease inhibitors against uPA, tPA, HTRA1, PACE4, inhibitors of the complement system, inhibitors against C1s, C1r, C3, C5 or Membrane attack complex, inhibitors againts proinflammtory cytokines, interleukin 1 converting enzyme, recombinant Interleukin 1 receptor antagonist, inhibitors against proinflammatory intracellular signaling pathways or p38-pathways, inhibitors of FAK-signaling, inhibitors against Toll-like receptors, doxicycline, glucosamin-hydrochloride, chondroitin sulfate, inhibitors of canonical WNT signaling, inhibitors of Frizzled receptors, modulators of GSK3β, inhibitors of SGK-1, recombinant Wif1, recombinant SFRP, recombinant DKK-1, inhibitors of LRP5 or LRP6, recombinant Sost, inhibitors of chondrocyte hypertrophic differentiation, HDAC-4 modulators, FGFR3 agonists, recombinant PTHrP, chondrocyte anabolic molecules, activators of TGFβ-Smad2-/3 signaling, activators of BMP-/Samd1,-5-,-8 signaling, modulators of Notch signaling, tyrpsone kinase inhibitors, Syndecan-4 inhibitors, inhibitors of leptin or leptin signaling, therapeutic principles with primary symptomatic treatment, inhibitors of TRPV1, inhibitors of P2X7, inhibitors of Nav1.7, inhibitors of PGE2-formation, non-steroidal anti-inflammatory drugs, corticosteroids, inhibitors of TrkA and COX2 inhibitors.

The compound useful for treatment of osteoarthritis, complications related to osteoarthritis or cartilage related disorders can be a protein, peptide, stapled peptide, antibody, nanobody, llama antibody, aptamer, small molecule, prodrug of a small molecule or an alternative format.

The compound useful for treatment of osteoarthritis, complications related to osteoarthritis or cartilage related disorders is selected from an inhibitor of MMP13 (such as PG-116800), recombinant metalloproteinase inhibitor-3 (TIMP-3), ADAMTS4/5 (such as AGG-523, US Patent Application Publication No. 2007/0043066), Cathepsin D (such as pepstatin A), Cathepsin K inhibitors (like ONO-5334 or odanacatib), inhibitors of Cathepsin S or B (such as Ac-Leu-Val-lysinal, CA-074, VBY-376), serine protease inhibitors against uPA, tPA, HTRA1, PACE4, inhibitors of the complement system (such as recombinant C1-inhibitor protein), inhibitors against C1s, C1r, C3, C5 or Membrane attack complex (MAC including C5b-9), inhibitors againts proinflammtory cytokines (such as IL1β or TNFalpha), interleukin 1 converting enzyme (ICE), recombinant Interleukin 1 receptor antagonist, inhibitors against proinflammatory intracellular signaling pathways (such as NfkappaB) or p38-pathways, inhibitors of FAK-signaling, inhibitors against Toll-like receptors (TLR-2 or TLR-4), doxicycline, glucosamin-hydrochloride, chondroitin sulfate, inhibitors of canonical WNT signaling (WNT-β-catenin) (such as inhibitors of WNT-molecules (e.g. Wnt3a, Wnt10b)), inhibitors of Frizzled receptors, modulators of GSK3β, inhibitors of SGK-1, recombinant Win, recombinant SFRP (FrzB), recombinant DKK-1, inhibitors of LRP5 or LRP6, recombinant Sost, inhibitors of chondrocyte hypertrophic differentiation (such as inhibitors of Hif2alpha, Runx2, Indian hedgehog, sirtuin-1, CXCR4, CXCR7, or GPER), HDAC-4 modulators, FGFR3 agonists, recombinant PTHrP, chondrocyte anabolic molecules (such as recombinant FGF18, recombinant FGF2, BMP-2 BMP-4, BMP-6, BMP-7, GDF5, TGFβ1, -2, -3), activators of TGFβ-Smad2-/3 signaling, activators of BMP-/Samd1, -5-, -8 signaling, modulators of Notch signaling, tyrpsone kinase inhibitors (such as DDR-1 or DDR2), Syndecan-4 inhibitors, inhibitors of leptin or leptin signaling, therapeutic principles with primary symptomatic treatment (amelioration of pain), inhibitors of TRPV1 (such as capsaicin), inhibitors of P2X7, inhibitors of Nav1.7, inhibitors of PGE2-formation, NSAIDs (non-steroidal anti-inflammatory drugs) (such as acetaminophen diclofenac, ibuprofen, fentaynl, ibuprofen, indomethacin), corticosteroids such as triamcinolone, inhibitors of TrkA and COX2 inhibitors (such as Celecoxib or Rofecoxib).

In some embodiments, the compound useful for treatment of osteoarthritis, complications related to osteoarthritis or cartilage related disorders is selected from corticosteroids, COX2 inhibitors, such as Celecoxib or Rofecoxib, or other nonsteroidal anti-inflammatory drugs, MMP-13 inhibitors, ADAMTS4/5 inhibitors such as Agg-523, cathepsin-B-inhibitors such as Ac-Leu-Val-lysinal, CA-074, VBY-376 or Cathepsin D inhibitors such as Pepstatin, Cathepsin K inhibitors such as ONO-5334 or odanacatib, inhibitors of PGE₂ synthesis.

A conservative substitution of the polypeptide means that at least one amino acid is replaced with another one without compromising the function of the polypeptide, which is to specifically bind to cartilage tissue.

The term cleavable linkers describes linker systems, being cleaved either by enzymatic processing e.g. by the action of proteases on said linker or nucleophilic attack by sulfur or oxygen containing residues or hydrolysis due to changes in pH or by irradiation, thereby liberating higher concentration of drug at the site of interest. Examples for cleavable linkers are known by the skilled man in the art and are described in the literature, e.g. G. Leriche et al., Bioorg. Med. Chem. 20 (2012) 571-582 and references cited therein.

Cleavable linker can also be selected from linker groups as indicated in the table 1 below.

TABLE 1

| Cleavage conditions | Cleavable groups |
|---|---|
| Enzymes | Residues cleavable by TEV, trypsin, thrombin, cathepsin B, |
| | cathespin D, cathepsin K, caspase 1, matrix |
| | metalloproteinasesequences, phosphodiester, phospholipid, ester, |
| | b-galactose2 |
| Nucleophilic/ | Dialkyl dialkoxysilane, cyanoethyl group, sulfone, ethylene |
| basic reagents | glycolyl disuccinate, 2-N-acyl nitrobenzenesulfonamide, a- |
| | thiophenylester, unsaturated vinyl sulfide, sulfonamide after |
| | activation, malondialdehyde (MDA)-indole derivative, levulinoyl |
| | ester, hydrazone, acylhydrazone, alkyl thioester |
| Reducing reagents | Disulfide bridges, azo compounds |
| Photo-irradiation | 2-Nitrobenzyl derivatives, phenacyl ester, 8-quinolinyl |
| | benzenesulfonate, coumarin, phosphotriester, bis-arylhydrazone, |
| | bimane bi-thiopropionic acid derivative |
| Electrophilic/ | Paramethoxybenzyl derivative, tert-butylcarbamate analogue, |
| acidic reagents | dialkyl or diaryl dialkoxysilane, orthoester, acetal, aconityl, |
| | hydrazone, b-thiopropionate, phosphoramidate, imine, trityl, vinyl |
| | ether, polyketal, alkyl 2-(diphenylphosphino)benzoate derivatives |
| Organometallic | Allyl ester, 8-hydroxyquinoline ester, picolinate ester |
| and metal | |
| catalyst | |
| Oxidizing | Vicinal diols, selenium compounds |
| reagents | |

Cleavable linker can also be selected from acid-lable linker systems such as tert-butyloxycarbonyl, paramethoxybenzyl, dialkyl or diaryldialkoxysylane, orthoester, acetal, b-thioproponate, ketal, phosphoamidate, hydrazone, vinyl ether, imine, aconityl, trityl, polyketal and such as linker exemplified in scheme 8 of Leriche et al., Bioorg. Med. Chem. 20 (2012) 571-582.

Cleavable linker can also be selected from photocleavable systems such as ortho-nitrobenzyl derivatives, phenacyl ester derivatives, and others photocleavable linkers such as ortho-notribenzyl based linker, phenacyl ester based linker, 8-quinolinyl benzenesulfonate linker, dicoumarin linker, 6-bromo-7-alkoxycoumarin-4-ylmethoxycarbonyl, bimane based linker, bis-arylhydrazone based linker such as linker exemplified in scheme 8 of Leriche et al., Bioorg. Med. Chem. 20 (2012) 571-582.

Cleavable linker can be selected from P. J. Jaun et al, Angew Chem Int Ed Engl. 2013 Jan. 28; 52(5):1404-9.

The term fluorophores describes compounds out of the group dimethylaminocoumarin derivative, preferably 7-dimethylaminocoumarin-4-acetic acid succinimidyl ester, Dansyl, 5/6-carboxyfluorescein and tetramethylrhodamine, BODIPY-493/503, BODIPY-FL, BODIPY-TMR, BODIPY-TMR-X, BODIPY-TR-X, BODIPY630/550-X, BODIPY-650/665-X, Alexa 350, Alexa 488, Alexa 532, Alexa 546, Alexa 555, Alexa 635, Alexa 647, Cyanine 3 (Cy 3), Cyanine 3B (Cy 3B), Cyanine 5 (Cy 5), Cyanine 5.5 (Cy 5.5), Cyanine 7 (Cy 7), Cyanine 7.5 (Cy 7.5), ATTO 488, ATTO 532, ATTO 600, ATTO 655, DY-505, DY-547, DY-632, DY-647; Fluorescent proteins, such as green fluorescent protein (GFP) and modifications of GFP that have different absorption/emission properties are also useful. Complexes of certain rare earth metals (e.g., europium, samarium, terbium or dysprosium) are used in certain contexts, as are fluorescent nanocrystals (quantum dots). Most preferred is a fluorophore selected from the group consisting of a dimethylaminocoumarin derivative, preferably 7-dimethyl aminocoumarin-4-acetic acid succinimidyl ester, Dansyl, 5/6-carboxyfluorescein and tetramethylrhodamine, BODIPY-493/503, BODIPY-FL, BODIPY-TMR, BODIPY-TMR-X, BODIPY-TR-X, BODIPY630/550-X, BODIPY-650/665-X, Alexa 350, Alexa 488, Alexa 532, Alexa 546, Alexa 555, Alexa 635, Afexa 647, Cyanine 3 (Cy 3), Cyanine 3B (Cy 3B), Cyanine 5 (Cy 5), Cyanine 5.5 (Cy 5.5), Cyanine 7 (Cy 7), Cyanine 7.5 (Cy 7.5), ATTO 488, ATTO 532, ATTO 600, ATTO 655, DY-505, DY-547, DY-632, DY-647] Preferred examples of optical imaging moieties are the cyanine dyes out of the group Carbacyanines, Oxacyanines, Thiacyanines and Azacyanines. Cyanine dyes are compounds defined by a polyene chain containing an odd number of carbon atoms linked by alternating single and multiple, preferably double, carbon-carbon bonds, terminated at either end by an amino group, one of which is quatemised. The cyanine and analogues aryl-linker-aryl chromophores optionally carry pendant or fused ring substituents. The cyanine dyes are particularly useful due to the wide range of spectral properties and structural variations available. A range of cyanine dyes are well known and tested, they have low toxicity, and are commercially available. The cyanine dyes are a single family of highly intense dyes with good aqueous solubility. They are pH insensitive between pH 3-10, exhibit low non-specific binding, and are more photostable than fluorescein.

The term ABP (activity-based probe) refers to a residue containing two suitable fluorophores attached in a distance suitable for fluorescence energy resonance transfer (FRET) and linked by a ABP cleavable linker. The ABP cleavable linker is a peptide consisting of two to 12 amino acids and containing cleavage sequences recognized by therapeutically relevant proteases like MMP-13, ADAMTS4/5, CathD, or other relvant proteins and may be substituted by or attached via C₁-C₁₆-alkyl, poly-ethyleneglycol chains, ethers, alkylamines or other residues.

According to another embodiment when Z1 is a fluorophore, the ABP may consist of a second fluorophore attached in a distance allowing FRET and linked by a ABP cleavable linker as defined above, such as upon cleavage of said linker system fluorescence intensity of the system changes.

Alternatively, the ABP may contain a reactive capture group like AOMK (AcylOxyMethylKetone), an epoxide, fluoroketone or similar known to the one skilled in the art that can react with the active center of for example a protease of interest e.g. with a reactive cycteine to form a new covalent bond, thereby labeling said enzyme and attaching one part of the molecule (either containing Z or the DOTA) to the target protein.

As an example, when Y1, Y2, Y3 and Y4 represent a group —(C₀-C₄)-alkyl-C(O)—N(R1)-, such as —CO—NH—, it should be understood that the groups are linked respectively to L1, L2, L3 and L4 and Z1, Z2, Z3 and Z4 as follows:

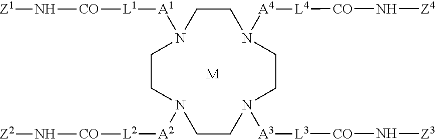

The same apply to all other definitions of all other substituents.

As another example, when Z1, Z2, Z3 and Z4 represent a polypeptide WYRGRL-, it should be understood that the groups are linked respectively to Y1, Y2, Y3 and Y4 as follows:

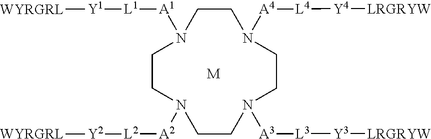

The present invention comprises all stereoisomeric forms of the compounds of the formula (I), (Ia) and (Ib) for example all enantiomers and diastereomers including cis/trans isomers. The invention likewise comprises mixtures of two or more stereoisomeric forms, for example mixtures of enantiomers and/or diastereomers including cis/trans isomers, in all ratios. Asymmetric centers contained in the compounds of the formula (I), (Ia) and (Ib) can all independently of each other have S configuration or R configuration. The invention relates to enantiomers, both the levorotatory and the dextrorotatory antipode, in enantiomerically pure form and essentially enantiomerically pure form, and in the form of their racemate, i.e. a mixture of the two enantiomers in molar ratio of 1:1, and in the form of mixtures of the two enantiomers in all ratios. The invention likewise relates to diastereomers in the form of pure and essentially pure diastereomers and in the form of mixtures of two or more diastereomers in all ratios. The invention also comprises all cis/trans isomers of the compounds of the formula (I), (Ia) and (Ib) in pure form and essentially pure form, and in the form of mixtures of the cis isomer and the trans isomer in all ratios. Cis/trans isomerism can occur in substituted rings. The preparation of individual stereoisomers, if desired, can be carried out by resolution of a mixture according to customary methods, for example, by chromatography or crystallization, or by use of stereochemically uniform starting compounds in the synthesis, or by stereoselective reactions. Optionally, before a separation of stereoisomers a derivatization can be carried out. The separation of a mixture of stereoisomers can be carried out at the stage of the compound of the formula (I), (Ia) and (Ib) or at the stage of an intermediate in the course of the synthesis. For example, in the case of a compound of the formula (I), (Ia) and (Ib) containing an asymmetric center the individual enantiomers can be prepared by preparing the racemate of the compound of the formula (I), (Ia) and (Ib) and resolving it into the enantiomers by high pressure liquid chromatography on a chiral phase according to standard procedures, or resolving the racemate of any intermediate in the course of its synthesis by such chromatography or by crystallization of a salt thereof with an optically active amine or acid and converting the enantiomers of the intermediate into the enantiomeric forms of the final compound of the formula (I), (Ia) and (Ib), or by performing an enantioselective reaction in the course of the synthesis. The invention also comprises all tautomeric forms of the compounds of the formula (I), (Ia) and (Ib).

Besides the free compounds of the formula (I), (Ia) and (Ib), i.e. compounds in which acidic and basic groups are not present in the form of a salt, the present invention comprises also the physiologically or toxicologically acceptable salts of the compounds of the formula (I), (Ia) and (Ib), especially their pharmaceutically acceptable salts, which can be formed on one or more acidic or basic groups in the compounds of the formula (I), (Ia) and (Ib), for example on basic heterocyclic moieties. The compounds of the formula (I), (Ia) and (Ib) may thus be deprotonated on an acidic group by an inorganic or organic base and be used, for example, in the form of the alkali metal salts. Compounds of the formula (I), (Ia) and (Ib) comprising at least one basic group may also be prepared and used in the form of their acid addition salts, for example in the form of pharmaceutically acceptable salts with inorganic acids and organic acids, such as salts with hydrochloric acid and thus be present in the form of the hydrochlorides, for example. Salts can in general be prepared from acidic and basic compounds of the formula (I), (Ia) and (Ib) by reaction with an acid or base in a solvent or diluent according to customary procedures. If the compounds of the formula (I), (Ia) and (Ib) simultaneously contain an acidic and a basic group in the molecule, the invention also includes internal salts (betaines, zwitterions) in addition to the salt forms mentioned. The present invention also comprises all salts of the compounds of the formula (I), (Ia) and (Ib) which, because of low physiological tolerability, are not directly suitable for use as a pharmaceutical, but are suitable as intermediates for chemical reactions or for the preparation of physiologically acceptable salts, for example by means of anion exchange or cation exchange.

The present invention also comprises different protonic states of the compounds of the formula (I), (Ia) and (Ib) existing under e.g. physiological conditions.

Another subject of the present invention is the compounds of the formula (Ia), in any of their stereoisomeric forms or a mixture of stereoisomeric forms in any ratio, and the pharmaceutically acceptable salts thereof,

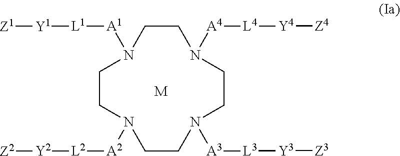

wherein

M is absent or present and a positively charged Gd metal ion;

A1, A2, A3 and A4 are independently of one another, identical or different, and are —(C₀-C₄)-alkyl-C(O)—N(R1)-;

L1, L2, L3 and L4 are independently of one another, identical or different, and are —(CH₂)ₘ[—O—(CH₂)ₚ]_{q}—, wherein m, q and p are independently of one another identical or different and are the integers zero, 1, 2, 3, 4 and 5;

Y1, Y2, Y3 and Y4 are independently of one another, identical or different, and are independently of one another selected from —(C₀-C₄)-alkyl-N(R1)-, —N(R1)-C(O)—(C₀-C₆)-alkyl-;

R1 is independently of one another selected from hydrogen and (C₁-C₄)-alkyl;

Z1, Z2, Z3 and Z4 are independently of one another, identical or different, and are independently of one another selected from:
a hydrogen atom;
a polypeptide comprising an amino acid sequence of WYRGRL-(SEQ ID NO:1) [TrpTyrArgGlyArgLeu] SEQ ID NO:I, or a conservative substitution or a repetitive sequence thereof, wherein the polypeptide specifically binds to cartilage tissue, the N-terminal moiety of the polypeptide being acetylated;
a fluorophore suitable for optical imaging;
an activity based probe (ABP) suitable to monitor the aberrant expression or activity of proteins involved in the initiation and progression of OA;
with the proviso that
at least one of Z1, Z2, Z3 and Z4 represent a polypeptide comprising an amino acid sequence of WYRGRL-(SEQ ID NO:1) [TrpTyrArgGlyArgLeu] SEQ ID NO:I, or a conservative substitution or a repetitive sequence thereof, wherein the polypeptide specifically binds to cartilage tissue, the N-terminal moiety of the polypeptide being acetylated;
at least one of Z1, Z2, Z3 and Z4 represent a fluorophore suitable for optical imaging or an activity based probe (ABP) suitable to monitor the aberrant expression or activity of proteins involved in the initiation and progression of OA;
Z1, Z2, Z3 and Z4 cannot represent more than one activity based probe (ABP) suitable to monitor the aberrant expression or activity of proteins involved in the initiation and progression of OA.

Another subject of the present invention is the compounds of the formula (Ib), in any of their stereoisomeric forms or a mixture of stereoisomeric forms in any ratio, and the pharmaceutically acceptable salts thereof.

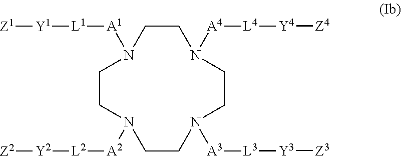

wherein

A1, A2, A3 and A4 are independently of one another, identical or different, and are —(C₀-C₄)-alkyl-C(O)—N(R1)-;

L1, L2, L3 and L4 are independently of one another, identical or different, and are —(CH₂)ₘ[—O—(CH₂)ₚ]_{q}—, wherein m, q and p are independently of one another identical or different and are the integers zero, 1, 2, 3, 4 and 5;

Y1, Y2, Y3 and Y4 are independently of one another, identical or different, and are independently of one another selected from —(C₀-C₄)-alkyl-N(R1)-, —N(R1)-C(O)—(C₀-C₆)-alkyl-;

R1 is independently of one another selected from hydrogen and (C₁-C₄)-alkyl;

Z1, Z2, Z3 and Z4 are independently of one another, identical or different, and are independently of one another selected from:
a hydrogen atom;
a polypeptide comprising an amino acid sequence of WYRGRL-(SEQ ID NO:1) [TrpTyrArgGlyArgLeu] SEQ ID NO:I, or a conservative substitution or a repetitive sequence thereof, wherein the polypeptide specifically binds to cartilage tissue, the N-terminal moiety of the polypeptide being acetylated;
a compound useful for treatment of osteoarthritis, complications related to osteoarthritis or cartilage related disorders;
with the proviso that
at least one of Z1, Z2, Z3 and Z4 represent a polypeptide comprising an amino acid sequence of WYRGRL-(SEQ ID NO:1) [TrpTyrArgGlyArgLeu] SEQ ID NO:I, or a conservative substitution or a repetitive sequence thereof, wherein the polypeptide specifically binds to cartilage tissue, the N-terminal moiety of the polypeptide being acetylated;
at least one of Z1, Z2, Z3 and Z4 represent a compound useful for treatment of osteoarthritis, complications related to osteoarthritis or cartilage related disorders as defined above.

Another subject of the present invention is the compounds of the formula (Ib), in any of their stereoisomeric forms or a mixture of stereoisomeric forms in any ratio, and the pharmaceutically acceptable salts thereof, as described above wherein the compound useful for treatment of osteoarthritis, complications related to osteoarthritis or cartilage related disorders is selected from corticosteroids, COX2 inhibitors, nonsteroidal anti-inflammatory drugs, MMP-13 inhibitors, ADAMTS4/5 inhibitors, cathepsin-B-inhibitors, Cathepsin D inhibitors, Cathepsin K inhibitors and inhibitors of PGE₂ synthesis.

Another subject of the present invention is the compounds of the formula (Ia), in any of their stereoisomeric forms or a mixture of stereoisomeric forms in any ratio, and the pharmaceutically acceptable salts thereof,

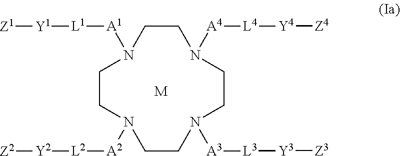

wherein

M is absent or present and a positively charged Gd metal ion;

A1, A2, A3 and A4 are identical and are —CH₂—C(O)—NH—;

L1, L2, L3 and L4 are identical and are —(CH₂)ₘ[—O—(CH₂)ₚ]_{q}—, wherein m, q and p are identical and are 2;

Y1, Y2, Y3 and Y4 are independently of one another, identical or different, and are independently of one another selected from —NH— and —NH—CO—(CH₂)₅—;

Z1, Z2, Z3 and Z4 are independently of one another, identical or different, and are independently of one another selected from:
a hydrogen atom;
a polypeptide comprising an amino acid sequence of WYRGRL-(SEQ ID NO:1) [TrpTyrArgGlyArgLeu] SEQ ID NO:I, or a conservative substitution or a repetitive sequence thereof, wherein the polypeptide specifically binds to cartilage tissue, the N-terminal moiety of the polypeptide being acetylated;
a fluorophore suitable for optical imaging;
an activity based probe (ABP) suitable to monitor the aberrant expression or activity of proteins involved in the initiation and progression of OA;
with the proviso that
at least one of Z1, Z2, Z3 and Z4 represent a polypeptide comprising an amino acid sequence of WYRGRL-(SEQ ID NO:1) [TrpTyrArgGlyArgLeu] SEQ ID NO:I, or a conservative substitution or a repetitive sequence thereof, wherein the polypeptide specifically binds to cartilage tissue, the N-terminal moiety of the polypeptide being acetylated;
at least one of Z1, Z2, Z3 and Z4 represent a fluorophore suitable for optical imaging or an activity based probe (ABP) suitable to monitor the aberrant expression or activity of proteins involved in the initiation and progression of OA;
Z1, Z2, Z3 and Z4 cannot represent more than one activity based probe (ABP) suitable to monitor the aberrant expression or activity of proteins involved in the initiation and progression of OA.

Another subject of the present invention is the compounds of the formula (Ib), in any of their stereoisomeric forms or a mixture of stereoisomeric forms in any ratio, and the pharmaceutically acceptable salts thereof,

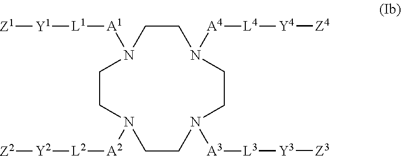

wherein

A1, A2, A3 and A4 are identical and are —CH₂—C(O)—NH—;

L1, L2, L3 and L4 are identical and are —(CH₂)ₘ[—O—(CH₂)ₚ]_{q}—, wherein m, q and p are identical and are 2;

Y1, Y2, Y3 and Y4 are independently of one another, identical or different, and are independently of one another selected from —NH— and —NH—CO—(CH₂)₅—;

Z1, Z2, Z3 and Z4 are independently of one another, identical or different, and are independently of one another selected from:
a hydrogen atom;
a polypeptide comprising an amino acid sequence of WYRGRL-(SEQ ID NO:1) [TrpTyrArgGlyArgLeu] SEQ ID NO:I, or a conservative substitution or a repetitive sequence thereof, wherein the polypeptide specifically binds to cartilage tissue, the N-terminal moiety of the polypeptide being acetylated;
a compound useful for treatment of osteoarthritis, complications related to osteoarthritis or cartilage related disorders;
with the proviso that
at least one of Z1, Z2, Z3 and Z4 represent a polypeptide comprising an amino acid sequence of WYRGRL-(SEQ ID NO:1) [TrpTyrArgGlyArgLeu] SEQ ID NO:I, or a conservative substitution or a repetitive sequence thereof, wherein the polypeptide specifically binds to cartilage tissue, the N-terminal moiety of the polypeptide being acetylated;
at least one of Z1, Z2, Z3 and Z4 represent a compound useful for treatment of osteoarthritis, complications related to osteoarthritis or cartilage related disorders as defined above.

Another subject of the present invention is the compounds of the formula (Ib), in any of their stereoisomeric forms or a mixture of stereoisomeric forms in any ratio, and the pharmaceutically acceptable salts thereof, as described above wherein the compound useful for treatment of osteoarthritis, complications related to osteoarthritis or cartilage related disorders is selected from corticosteroids, COX2 inhibitors, nonsteroidal anti-inflammatory drugs, MMP-13 inhibitors, ADAMTS4/5 inhibitors, cathepsin-B-inhibitors, Cathepsin D inhibitors, Cathepsin K inhibitors and inhibitors of PGE₂ synthesis.

Another subject of the present invention is the compounds of the formula (Ia), in any of their stereoisomeric forms or a mixture of stereoisomeric forms in any ratio, and the pharmaceutically acceptable salts thereof,

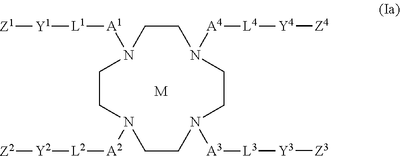

wherein

M is absent or present and a positively charged Gd metal ion;

A1, A2, A3 and A4 are identical and are —CH₂—C(O)—NH—;

L1, L2, L3 and L4 are identical and are —(CH₂)ₘ[—O—(CH₂)ₚ]_{q}—, wherein m, q and p are identical and are 2;

Y1, Y2, Y3 and Y4 are independently of one another, identical or different, and are independently of one another selected from —NH— and —NH—CO—(CH₂)₅—;

Z1, Z2, Z3 and Z4 are independently of one another, identical or different, and are independently of one another selected from:
a hydrogen atom,
a polypeptide sequence of WYRGRL-(SEQ ID NO:1) [TrpTyrArgGlyArgLeu] SEQ ID NO:I, the N-terminal moiety of the polypeptide being acetylated;
a fluorophore suitable for optical imaging;

with the proviso that
at least one of Z1, Z2, Z3 and Z4 represent a polypeptide comprising an amino acid sequence of WYRGRL-(SEQ ID NO:1) [TrpTyrArgGlyArgLeu] SEQ ID NO:I, the N-terminal moiety of the polypeptide being acetylated;
at least one of Z1, Z2, Z3 and Z4 represent a fluorophore suitable for optical imaging.

Another subject of the present invention is the compounds of the formula (Ia) as described above wherein fluorophore suitable for optical imaging is Cyanine 5.5.

Another subject of the present invention is the compounds of the formula (Ib), in any of their stereoisomeric forms or a mixture of stereoisomeric forms in any ratio, and the pharmaceutically acceptable salts thereof,

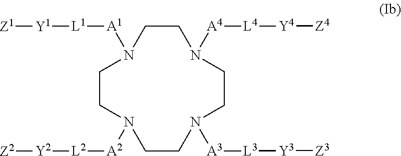

wherein

A1, A2, A3 and A4 are identical and are —CH₂—C(O)—NH—;

L1, L2, L3 and L4 are identical and are —(CH₂)ₘ[—O—(CH₂)ₚ]_{q}—, wherein m, q and p are identical and are 2;

Y1, Y2, Y3 and Y4 are independently of one another, identical or different, and are independently of one another selected from —NH— and —NH—CO—(CH₂)₅—;

Z1, Z2, Z3 and Z4 are independently of one another, identical or different, and are independently of one another selected from:
a hydrogen atom,
a polypeptide sequence of WYRGRL-(SEQ ID NO:1) [TrpTyrArgGlyArgLeu] SEQ ID NO:I, the N-terminal moiety of the polypeptide being acetylated;
an inhibitor of Cathepsin D.

with the proviso that:
at least one of Z1, Z2, Z3 and Z4 represent a polypeptide comprising an amino acid sequence of WYRGRL-(SEQ ID NO:1) [TrpTyrArgGlyArgLeu] SEQ ID NO:I, the N-terminal moiety of the polypeptide being acetylated;
at least one of Z1, Z2, Z3 and Z4 represent an inhibitor of Cathepsin D.

Another subject of the present invention is the compounds of the formula (Ib) as described above wherein the inhibitor of Cathepsin D is Pepstatin A.

Other subjects of the present invention are processes for the preparation of the compounds of the formula I which are outlined below and by which the compounds of the formula I and intermediates and occurring in the course of their synthesis, and salts thereof, are obtainable. The compounds of the formula I can be prepared by utilizing procedures and techniques which per se are known to a person skilled in the art.

Peptide Synthesis

The skilled person is aware of a variety of different methods to prepare peptides that are described in this invention. These methods include but are not limited to synthetic approaches and recombinant gene expression. Thus, one way of preparing these peptides is the synthesis in solution or on a solid support and subsequent isolation and purification. A different way of preparing the peptides is gene expression in a host cell in which a DNA sequence encoding the peptide has been introduced. Alternatively, the gene expression can be achieved without utilizing a cell system. The methods described above may also be combined in any way.

A preferred way to prepare the peptides of the present invention is solid phase synthesis on a suitable resin. Solid phase peptide synthesis is a well established methodology (see for example: Stewart and Young, Solid Phase Peptide Synthesis, Pierce Chemical Co., Rockford, Ill., 1984; E. Atherton and R. C. Sheppard, Solid Phase Peptide Synthesis. A Practical Approach, Oxford-IRL Press, New York, 1989). Solid phase synthesis is initiated by attaching an N-terminally protected amino acid with its carboxy terminus to an inert solid support carrying a cleavable linker. This solid support can be any polymer that allows coupling of the initial amino acid, e.g. a trityl resin, a chlorotrityl resin, a Wang resin or a Rink resin in which the linkage of the carboxy group (or carboxamide for Rink resin) to the resin is sensitive to acid (when Fmoc strategy is used). The polymer support must be stable under the conditions used to deprotect the a-amino group during the peptide synthesis.

After the first amino acid has been coupled to the solid support, the a-amino protecting group of this amino acid is removed. The remaining protected amino acids are then coupled one after the other in the order represented by the peptide sequence using appropriate amide coupling reagents, for example BOP (benzotriazol-1-yl-oxy-tris-(dimethylamino)-phosphonium), HBTU (2-(1H-benzotriazol-1-yl)-1,1,3,3-tetramethyl-uronium), HATU (O-(7-azabenztriazol-1-yl-oxy-tris-(dimethylamino)-phosphonium) or DIC (N,N′-diisopropylcarbodiimide)/HOBt (1-hydroxybenzotriazol), wherein BOP, HBTU and HATU are used with tertiary amine bases. Alternatively, the liberated N-terminus can be functionalized with groups other than amino acids, for example carboxylic acids, etc.

Usually, reactive side-chain groups of the amino acids are protected with suitable blocking groups. These protecting groups are removed after the desired peptides have been assembled. They are removed concomitantly with the cleavage of the desired product from the resin under the same conditions. Protecting groups and the procedures to introduce protecting groups can be found in Protective Groups in Organic Synthesis, 3d ed., Greene, T. W. and Wuts, P. G. M., Wiley & Sons (New York: 1999).

In some cases it might be desirable to have side-chain protecting groups that can selectively be removed while other side-chain protecting groups remain intact. In this case the liberated functionality can be selectively functionalized. For example, a arginine may be protected with an Pmc protecting group which is labile to acidic conditions, for example trifluoroacetic acid in dicloromethane. Thus, if the N-terminal amino group and all side-chain functionalities are protected with nuclephilic base labile protecting groups, the Pmc 2,2,5,7,8-pentamethylchroman-6-sulfonyl) group can be selectively removed using trifluoroacetic acid in dicloromethane and the corresponding free guanidino group can then be further modified, e.g. by acylation. Finally the peptide is cleaved from the resin. This can be achieved by using King's cocktail (D. S. King, C. G. Fields, G. B. Fields, Int. J. Peptide Protein Res. 36, 1990, 255-266). The raw material can then be purified by chromatography, e.g. preparative RP-HPLC, if necessary.

Examples of protective groups which may be mentioned, are benzyl protective groups, for example benzyl ethers of hydroxy compounds and benzyl esters of carboxylic acids, from which the benzyl group can be removed by catalytic hydrogenation in the presence of a palladium catalyst, tert-butyl protective groups, for example tert-butyl esters of carboxylic acids, from which the tert-butyl group can be removed by treatment with trifluoroacetic acid, acyl protective groups, for example ester and amides of hydroxy compounds and amino compounds, which can be cleaved again by acidic or basic hydrolysis, or alkoxycarbonyl protective groups, for example tert-butoxycarbonyl derivatives of amino compounds, which can be cleaved again by treatment with trifluoroacetic acid. Compounds of the formula I can also be prepared by solid phase techniques. In such a synthetic approach, the solid phase may also be regarded as having the meaning of a protecting group, and cleavage from the solid phase as removal of the protective group. The use of such techniques is known to a person skilled in the art (cf. Burgess K (Ed.), Solid Phase Organic Synthesis, New York, Wiley, 2000). For example, a phenolic hydroxy group can be attached to a trityl-polystyrene resin, which serves as a protecting group, and the molecule cleaved from the resin by treatment with trifluoroacetic acid or another acid at a later stage of the synthesis.

As is usual and applies to all reactions performed in the course of the synthesis of a compound of the formula (I), (Ia) and (Ib), appropriate details of the conditions applied in a specific preparation process, including the solvent, a base or acid, the temperature, the order of addition, the molar ratios and other parameters, are routinely chosen by the skilled person in view of the characteristics of the starting compounds and the target compound and the other particularities of the specific case. As is also known by the skilled person, not all processes described herein will in the same way be suitable for the preparation of all compounds of the formula (I), (Ia) and (Ib) and their intermediates, and adaptations have to be made. In all processes for the preparation of the compounds of the formula (I), (Ia) and (Ib), workup of the reaction mixture and the purification of the product is performed according to customary methods known to the skilled person which include, for example, quenching of a reaction mixture with water, adjustment of a certain pH, precipitation, extraction, drying, concentration, crystallization, distillation and chromatography. As further examples of methods applicable in the synthesis of the compounds of the formula (I), (Ia) and (Ib), microwave assistance for speeding-up, facilitating or enabling reactions, as described by P. Lidstrom, J. Tierney, B. Wathey, J. Westman, Tetrahedron, 57(2001), 9225, for example, may be mentioned, and modern separation techniques like preparative high pressure liquid chromatography (HPLC), which can be used for separating mixtures of positional isomers which may occur in any reactions. Also for the characterization of the products customary methods are used such as NMR, IR and mass spectroscopy.

Another subject of the present invention are the novel starting compounds and intermediates occurring in the synthesis of the compounds of the formula (I), (Ia) and (Ib), defined as above, in any of their stereoisomeric forms or a mixture of stereoisomeric forms in any ratio, and their salts, and their use as synthetic intermediates or starting compounds. All general explanations, specifications of embodiments and definitions of numbers and groups given above with respect to the compounds of the formula (I), (Ia) and (Ib) apply correspondingly to the said intermediates and starting compounds. A subject of the invention are in particular the novel specific starting compounds and intermediates described herein. Independently thereof whether they are described as a free compound and/or as a specific salt, they are a subject of the invention both in the form of the free compounds and in the form of their salts, and if a specific salt is described, additionally in the form of this specific salt.

The compounds of the formula (I), (Ia) and (Ib) and their pharmaceutically acceptable salts can therefore be used in animals, in particular in mammals and specifically in humans, as a pharmaceutical or medicament on their own, in mixtures with one another, or in the form of pharmaceutical compositions. A subject of the present invention also is the compounds of the formula (I), (Ia) and (Ib) and their pharmaceutically acceptable salts for use as a pharmaceutical. A subject of the present invention also are pharmaceutical compositions and medicaments which comprise at least one compound of the formula (I), (Ia) and (Ib) and/or a pharmaceutically acceptable salt thereof as an active ingredient, in an effective dose for the desired use, and a pharmaceutically acceptable carrier, i.e. one or more pharmaceutically innocuous, or nonhazardous, vehicles and/or excipients, and optionally one or more other pharmaceutical active compounds.

Pharmaceutical formulations adapted for transdermal administration can be administered as plasters for extended, close contact with the epidermis of the recipient. For topical administration, formulations such as ointments, creams, suspensions, lotions, powders, solutions, pastes, gels, sprays, aerosols or oils can be used. For the treatment of the eye or other external tissue, for example mouth and skin, suitable formulations are topical ointments or creams, for example. In the case of ointments, the active ingredient can be employed either with a paraffinic or a water-miscible cream base. Alternatively, the active ingredient can be formulated to give a cream with an oil-in-water cream base or a water-in-oil base. Pharmaceutical formulations adapted for topical application to the eye include eye drops, in which the active ingredient is dissolved or suspended in a suitable carrier, in particular an aqueous solvent.

The pharmaceutical compositions according to the invention are prepared in a manner known per se and familiar to the person skilled in the art by admixing one ore more pharmaceutically acceptable inert inorganic and/or organic vehicles and excipients with one or more compounds of the formula (I), (Ia) and (Ib) and/or pharmaceutically acceptable salts thereof, and bringing them into a suitable form for dosage and administration, which can then be used in human medicine or veterinary medicine. For the production of pills, tablets, coated tablets and hard gelatin capsules it is possible to use, for example, lactose, cornstarch or derivatives thereof, talc, stearic acid or its salts. For the production of gelatin capsules and suppositories fats, waxes, semisolid and liquid polyols, natural or hardened oils, for example, can be used. For the production of solutions, for example injection solutions, or of emulsions or syrups water, saline, alcohols, glycerol, polyols, sucrose, invert sugar, glucose, vegetable oils, for example, can be used, and for the production of microcapsules, implants or rods copolymers of glycolic acid and lactic acid, for example, can be used. The pharmaceutical compositions normally contain from about 0.5% to 90% by weight of the compounds of the formula (I), (Ia) and (Ib) and/or their pharmaceutically acceptable salts. The amount of the active ingredient of the formula (I), (Ia) and (Ib) and/or its pharmaceutically acceptable salts in the pharmaceutical compositions normally is from about 0.5 mg to about 1000 mg, preferably from about 1 mg to about 500 mg per unit dose. Depending on the kind of the pharmaceutical composition and other particulars of the specific case, the amount may deviate from the indicated ones.

In addition to the active ingredients of the formula (I), (Ia) and (Ib) and/or their pharmaceutically acceptable salts and to vehicles, or carrier substances, the pharmaceutical compositions can contain excipients, or auxiliaries or additives, such as, for example, fillers, disintegrants, binders, lubricants, wetting agents, stabilizers, emulsifiers, preservatives, sweeteners, colorants, flavorings, aromatizers, thickeners, diluents, buffer substances, solvents, solubilizers, agents for achieving a depot effect, salts for altering the osmotic pressure, coating agents or antioxidants. They can also contain two or more compounds of the formula (I), (Ia) and (Ib), and/or their pharmaceutically acceptable salts. In case a pharmaceutical composition contains two or more compounds of the formula (I), (Ia) and (Ib), the selection of the individual compounds can aim at a specific overall pharmacological profile of the pharmaceutical composition. For example, a highly potent compound with a shorter duration of action may be combined with a long-acting compound of lower potency. The flexibility permitted with respect to the choice of substituents in the compounds of the formula (I), (Ia) and (Ib) allows a great deal of control over the biological and physico-chemical properties of the compounds and thus allows the selection of such desired compounds.

When using the compounds of the formula (I), (Ia) and (Ib), the dose can vary within wide limits and, as is customary and is known to the physician, is to be suited to the individual conditions in each individual case. It depends, for example, on the specific compound employed, on the nature and severity of the disease to be treated, on the mode and the schedule of administration, or on whether an acute or chronic condition is treated or whether prophylaxis is carried out. An appropriate dosage can be established using clinical approaches known to the person skilled in the art. In general, the daily dose for achieving the desired results in an adult weighing about 75 kg is from about 0.01 mg/kg to about 100 mg/kg, preferably from about 0.1 mg/kg to about 50 mg/kg, in particular from about 0.1 mg/kg to about 10 mg/kg, in each case in mg per kg of body weight. The daily dose can be divided, in particular in the case of the administration of relatively large amounts, into several, for example 2, 3 or 4, part administrations. As usual, depending on individual behavior it may be necessary to deviate upwards or downwards from the daily dose indicated.

The work leading to this invention has received funding from the European Union Seventh Framework Programme FP7-HEALTH-2009 under grant agreement number 241919.

The following examples illustrate the present invention.

When in the final step of the synthesis of an example compound an acid such as trifluoroacetic acid or acetic acid was used, for example when trifluoroacetic acid was employed to remove an acid-labile protecting group containing a tert-butyl group, or when a compound was purified by chromatography using an eluent which contained such an acid, in some cases, depending on the work-up procedure, for example the details of a freeze-drying process, the compound was obtained partially or completely in the form of a salt of the acid used, for example in the form of the salt with acetic acid salt or trifluoroacetic acid salt. In the names of the example compounds and the structural formulae such contained trifluoroacetic acid or acetic acid is not specified.

### ABBREVIATIONS

arom.=aromatic

Boc=tert-butyloxycarbonyl

DIPEA=diisopropyl-ethyl amine

DMF=dimethylformamide

DCM=dichloromethane

ESI-MS=electrospray ionisation mass spectrometry

equiv.=equivalents

Fmoc=9-fluorenylmethoxycarbonyl

HFIP=1,1,1,3,3,3-hexafluoro-2-propanol

HATU=2-(7-Aza-1H-benzotriazole-1-yl)-1,1,3,3-tetramethyluronium hexafluorophosphate.

HPLC=high performance liquid chromatography

LC-MS=liquid chromatography mass spectrometry

NMR=nuclear magnetic resonance

Pmc=2,2,5,7,8-pentamethylchroman-6-sulfonyl

TFA=trifluoro-acetic acid

### GENERAL METHODS

Unless otherwise noted, all reagents were purchased from commercial suppliers and used without, further purification. All solvents used were of HPLC grade. Reactions were analyzed LC-MS. Reverse-phase HPLC was performed on a C18 column Sun Fire 50×100 mm, Waters or XBridgeTMPrep C18, 5 μm (10×100 mm; Waters). LC/MS data were acquired using the Waters or HP-Agilent 1100 MSD system. NMR-data were recorded on a Bruker DRX-400 system in d₆-DMSO. Fluorescence assays was measured with a Tecan SAFIRE II spectrometer.

The prepared compounds were in general characterized by spectroscopic data and chromatographic data, in particular mass spectra (MS) and/or nuclear magnetic resonance (NMR) spectra. In the NMR characterization, the chemical shift δ (in ppm), the number of hydrogen atoms (H), the coupling constant J (in Hz) and the multiplicity (s: singlet, d: doublet, dd: double doublet, t: triplet, m: multiplet; br: broad) of the peaks are given. In the MS characterization, the mass number (m/e) of the peak of the molecular ion (M) or of a related ion such as the ion (M+1), i.e. the protonated molecular ion (M+H), or the ion (M−1), which was formed depending on the ionization method used, is given. Generally, the ionization method was electrospray ionization (ES+ or ES−).

General Procedures for Solid-Phase Peptide Synthesis

The peptides WYRGRL and YRLGRW were synthesized on solid resin using an automated peptide synthesizer (CEM Microwave Peptide Synthesizer) with standard F-moc chemistry. Both of the peptides were acetylated at the N terminus with a large excess of acetic anhydride and DIPEA. The fully protected peptide was cleaved from the resin using 30% HFIP in DCM and characterized by LC-MS.

Fmoc protected natural amino acids were purchased from Protein Technologies Inc., Senn Chemicals, Merck Biosciences, Novabiochem, Iris Biotech or Bachem. The following standard amino acids were used throughout the syntheses: Fmoc-Gly-OH, Fmoc-L-Trp(Boc)-OH, Fmoc-L-Tyr(tBu)-OH, Fmoc-L-Arg(Pmc)-OH, Fmoc-L-Leu-OH.

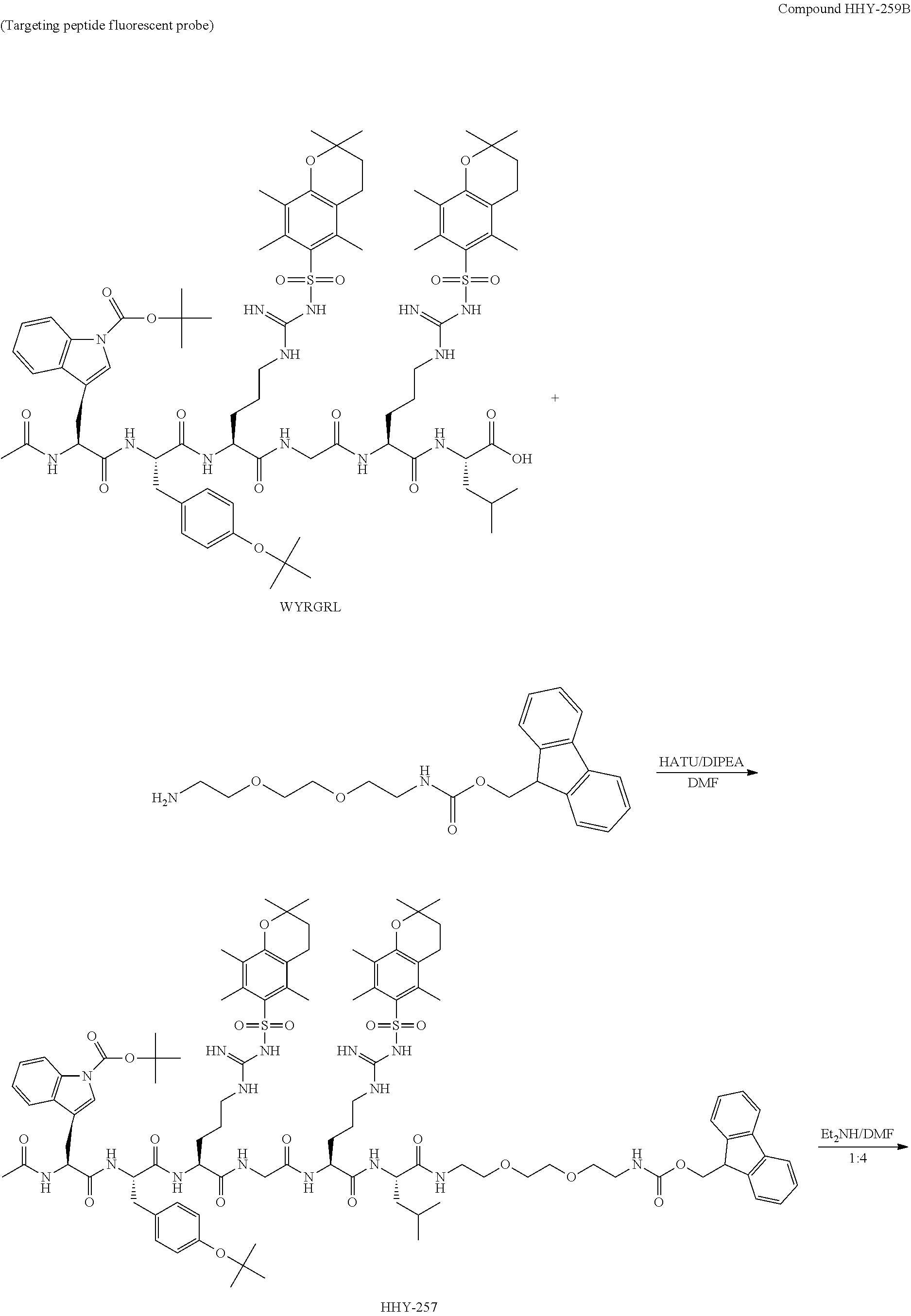

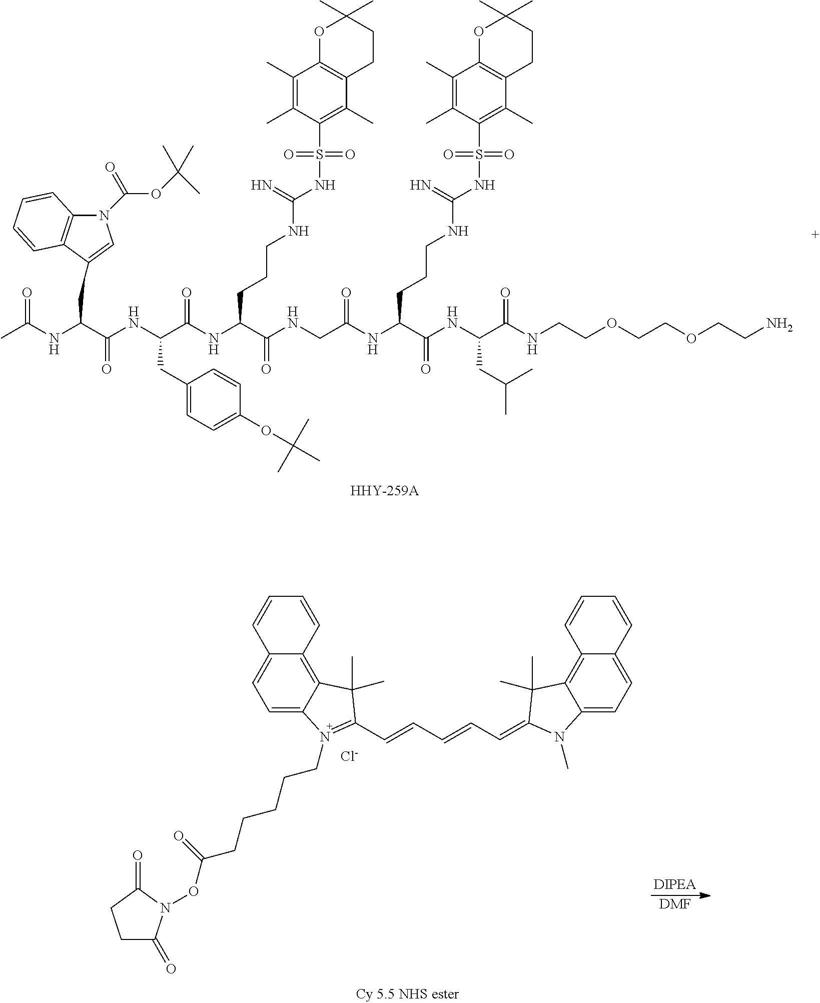

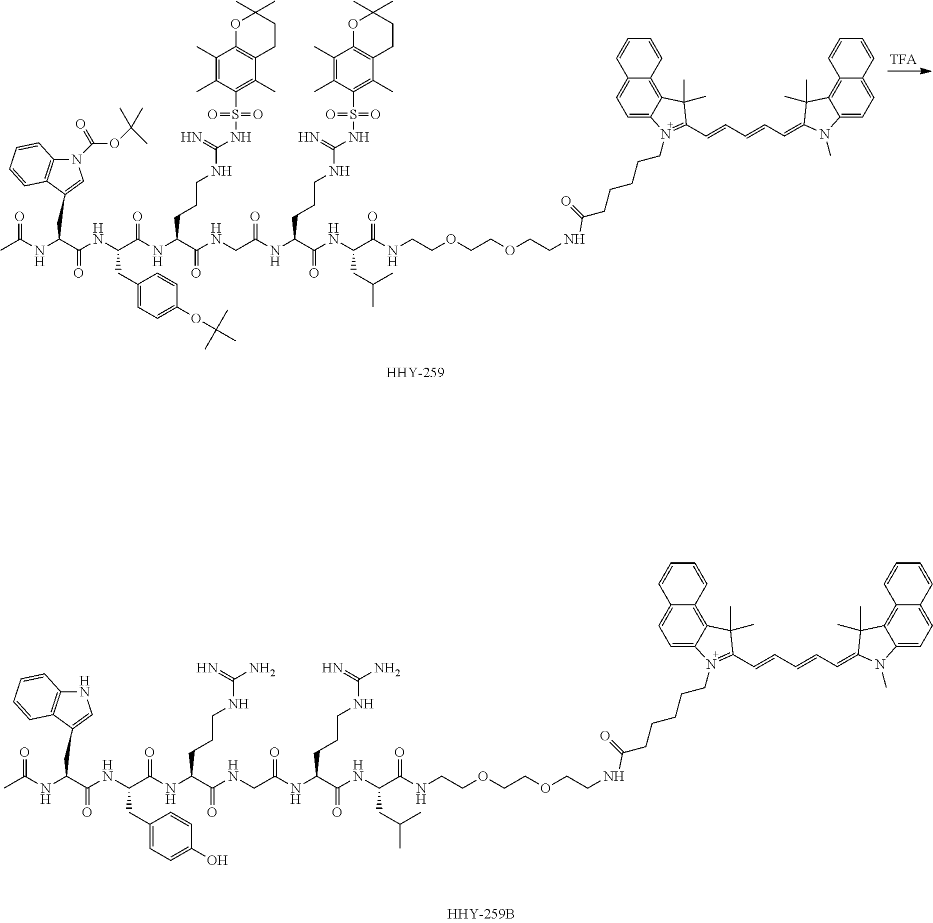

WYRGRL (32 mg, 20 μmol), 1 equiv. HATU (7.6 mg, 20 μmol) and 2 equiv. DIPEA (11 μl, 60 μmol) were solved in 2 ml DMF and stirred for 10 min. at room temperature. Subsequently 1 equiv. (8 mg, 20 μmol) 1-(9-Fluorenylmethyloxycarbonyl-amino)-3,6-dioxa-8-octaneamine hydrochloride was added to the reaction mixture, which was stirred under argon at room temperature for 1 h (LC-MS determination). The solution was diluted with EtOAc, and washed sequentially with 1N HCl, saturated NaHCO₃, and brine. The organic layer was dried over Na₂SO₄. After removal of the solvent in vacuo, the crude product HHY-257 was obtained as a pale yellow solid and was used in the next step without further purification. ESI-MS found: [M+2H]²⁺=967.5.

For removal of the Fmoc-group, HHY-257 (20 μmol) was dissolved in 2 ml Et₂NH/DMF (1/4) and the reaction mixture was stirred for 10 min. at room temperature. The solvent was removed under reduced pressure. The crude product HHY-259A was used without further purification in the next step. ESI-MS found: [M+2H]²⁺=856.6.

HHY-259A (20 μmol) was dissolved in 1.5 ml DMF, followed by the addition of 3 equiv. DIPEA (11 μl, 60 μmol) and 0.5 equiv. Cy5.5 NHS ester (7 mg, 10 μmol). The reaction mixture was stirred under argon at room temperature for 1 h (LC-MS determination). DMF was then removed under reduced pressure on a rotary evaporator. The crude product HHY-259 was used without further purification in the next step. ESI-MS found: [M+H]²⁺=1139.4.

For removal of all of the protection groups, the crude HHY-259 was dissolved in 2 ml 95% TFA/H₂O and the reaction mixture was stirred for 3 hours under argon at room temperature (LC-MS determination). The solvent was 3 times co-evaporated with toluene, purified by HPLC to give HHY-259B as a blue powder.

ESI-MS found: [M+H]²⁺=794.5.

Compound HHY-258B (Scrambled Peptide Fluorescent Probe)

The preparation of HEY-258B was performed under similar conditions as those described for probe HHY-259B using scrambled peptide sequence YRLGRW.

YRLGRW (32 mg, 20 μmol, 1 equiv. HATU (7.6 mg, 20 μmol) and 2 equiv. DIPEA (11 μl, 60 μmol) were solved in 2 ml DMF and stirred for 10 min. at room temperature. Subsequently 1 equiv. (8 mg, 20 μmol) 1-(9-Fluorenylmethyloxycarbonyl-amino)-3,6-dioxa-8-octaneamine hydrochloride was added to the reaction mixture, which was stirred under argon at room temperature for 1 h (LC-MS determination). The solution was diluted with EtOAc, and washed sequentially with 1N HCl, saturated NaHCO₃, and brine. The organic layer was dried over Na₂SO₄. After removal of the solvent in vacuo, the crude product HHY-256 was obtained as a pale yellow solid and was used in the next step without further purification. ESI-MS found: [M+2H]²⁺=967.3.

For removal of the Fmoc-group, HHY-256 (20 μmol) was dissolved in 2 ml Et₂NH/DMF (1/4) and the reaction mixture was stirred for 10 min. at room temperature. The solvent was removed under reduced pressure. The crude product HHY-258A was used without further purification in the next step.

ESI-MS found: [M+2H]²⁺=856.6.

HHY-258A (20 μmol) was dissolved in 1.5 ml DMF, followed by the addition of 3 equiv. DIPEA (11 μl, 60 μmol) and 0.5 equiv. Cy5.5 NHS ester (7 mg, 10 μmol). The reaction mixture was stirred under argon at room temperature for 1 h (LC-MS determination). DMF was then removed under reduced pressure on a rotary evaporator. The crude product HHY-258 was used without further purification in the next step. ESI-MS found: [M+H]²⁺=1138.9.

For removal of all of the protection groups, the crude HHY-258 was dissolved in 2 ml 95% TFA/H₂O and the reaction mixture was stirred for 3 hours under argon at room temperature (LC-MS determination). The solvent was 3 times co-evaporated with toluene, purified by HPLC to give HHY-258B as a blue powder.

ESI-MS found: [M+H]²⁺=794.2.

Synthesis of Key Intermediate for One-Peptide Conjugates

Compound HHY-343

Symmetrical di-protection of cyclen was performed by the reported procedure⁷. In brief, 2.0 g (11.6 mmol) of cyclen and 5.8 g (23.2 mmol) of Cbz-OSu was dissolved in CHCl₃ (40 mL), and the solution was stirred at room temperature for 2 days. After evaporation, the residue was suspended in 1 M NaOH, and the aqueous suspension was extracted by CH₂Cl₂ (40 mL×5). The organic layer was washed with brine, dried over K₂CO₃, and then evaporated to yield di-protected cyclen (5.1 g, 100%). This compound was used without further purification. ESI-MS calcd. for C₂₄H₃₂N₄O₄: 440.5. found: [M+H]⁺=441.3.

Compound HHY-344

To a mixture of compound HHY-343 (5.1 g, 11.6 mmol) and potassium carbonate (4.0 g, 29.0 mmol) in MeCN (60 mL) was added tert-butyl bromoacetate (4.12 g, 23.2 mmol). After refluxing overnight, the mixture was filtered through a pad of celite, and then evaporated. The residue was purified by flash chromatography (MeOH—CH₂Cl₂, 0-5%) to give compound HHY-344 (5.1 g, 64%) as a colorless gum. ESI-MS calcd. for C₃₆H₅₂N₄O₈: 668.8. found: [M+H]⁺=669.4.

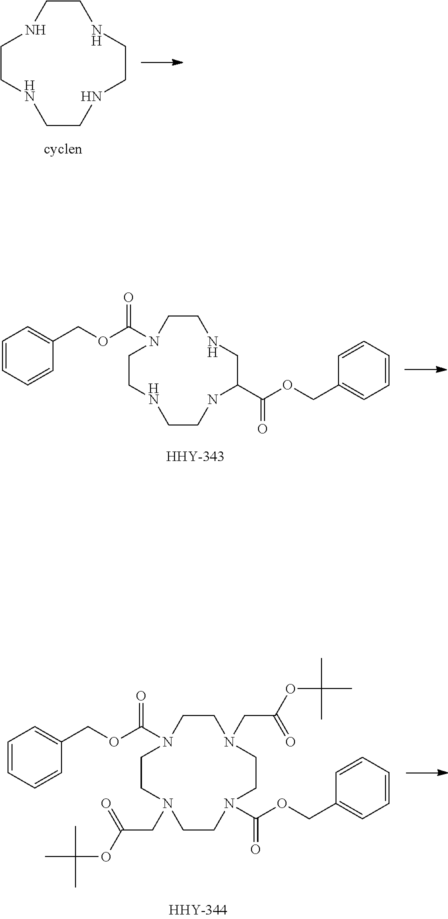 [SMILES: C1CNCCNCCNCCN1 ; O=C(OCc1ccccc1)C1CNCCN(C(=O)OCc2ccccc2)CCNCCN1 ; CC(C)(C)OC(=O)CN1CCN(C(=O)OCc2ccccc2)CCN(CC(=O)OC(C)(C)C)CCN(C(=O)OCc2ccccc2)CC1]

Compound HHY-345

A mixture of compound HHY-344 (5.1 g, 7.4 mmol) and 10% Pd/C (100 mg) in MeOH (40 mL) was stirred under hydrogen atmosphere at room temperature overnight. After filtration through a pad of celite, evaporation of the solvent gave compound HHY-345 (3.0 g, 100%) as a white solid. This compound was used without further purification. ESI-MS calcd. for C₂₀H₄₀N₄O₄: 400.5. found: [M+H]⁺=401.3.

Compound HHY-346

To a mixture of compound HHY-345 (660 mg, 1.5 mmol) and potassium carbonate (0.46 g, 3.4 mmol) in MeCN (30 mL) was added benzyl bromoacetate (0.69 g, 3 mmol). After refluxing overnight, the mixture was filtered through a pad of celite, and then evaporated. The residue was purified by flash chromatography (MeOH—CH₂Cl₂, 0-5%) to give compound HHY-346 (0.5 g, 50%) as a colorless gum. ESI-MS calcd. for C₃₈H₅₆N₄O₈: 696.9. found: [M+H]⁺=697.4.

Compound HHY-347

A mixture of compound HHY-346 (0.98 g, 1.4 mmol) and 10% Pd/C (50 mg) in MeOH (40 mL) was stirred under hydrogen atmosphere at room temperature overnight. After filtration through a pad of celite, evaporation of the solvent gave compound HHY-347 (0.73 g, 100%) as a white solid. This compound was used without further purification. ESI-MS calcd. for C₂₄H₄₄N₄O₈: 516.6. found: [M+H]⁺=517.4³.

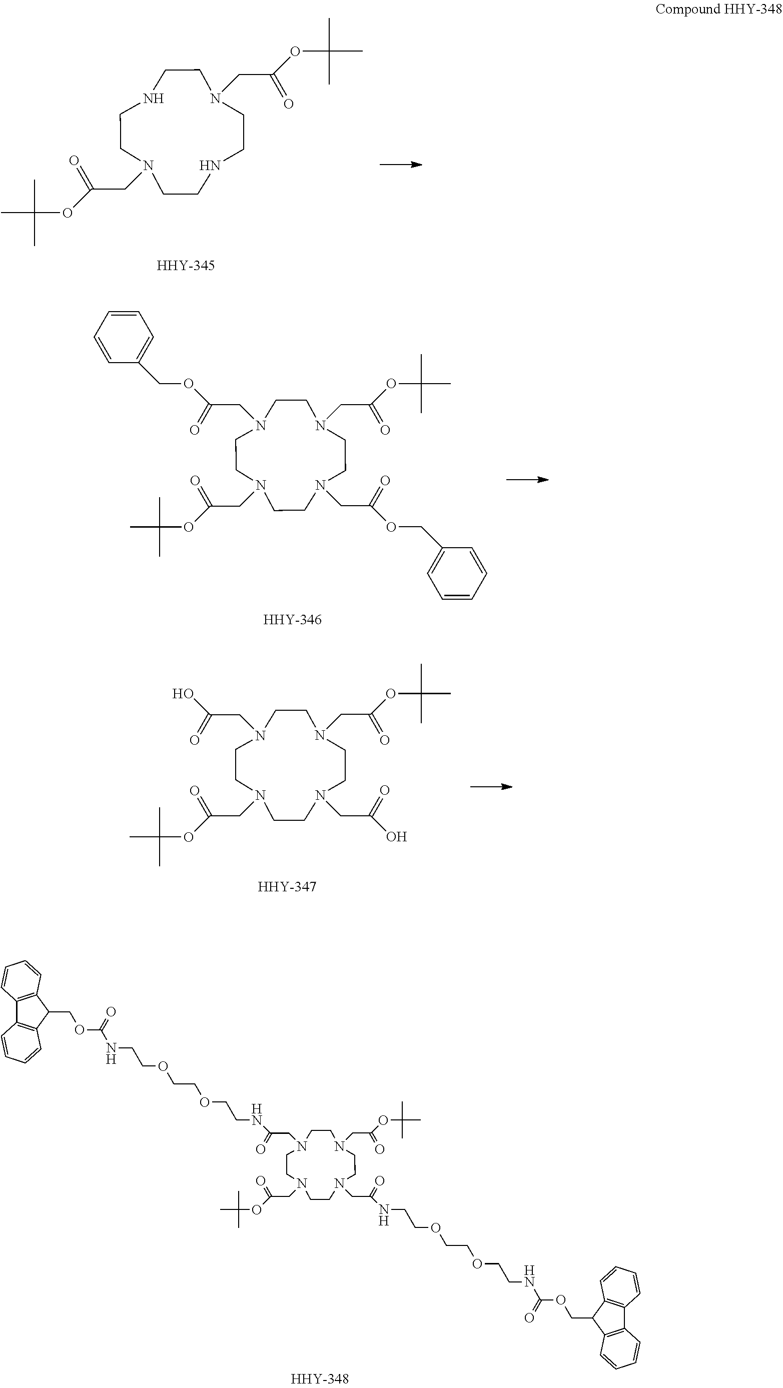 [SMILES: CC(C)(C)OC(=O)CN1CCNCCN(CC(=O)OC(C)(C)C)CCNCC1 ; CC(C)(C)OC(=O)CN1CCN(CC(=O)OCc2ccccc2)CCN(CC(=O)OC(C)(C)C)CCN(CC(=O)OCc2ccccc2)CC1 ; CC(C)(C)OC(=O)CN1CCN(CC(=O)O)CCN(CC(=O)OC(C)(C)C)CCN(CC(=O)O)CC1 ; CC(C)(C)OC(=O)CN1CCN(CC(=O)NCCOCCOCCNC(=O)OCC2c3ccccc3-c3ccccc32)CCN(CC(=O)OC(C)(C)C)CCN(CC(=O)NCCOCCOCCNC(=O)OCC2c3ccccc3-c3ccccc32)CC1]

1 equiv. compound HHY-347 (517 mg, 1 mmol), 2 equiv. HATU (760 mg, 1 mmol) and 8 equiv. DIPEA (1.4 mL, 8 mmol) were dissolved in 10 mL DMF. After 10 min, 1, equiv. 1-(9-Fluorenylmethyloxycarbonyl-amino)-3, 6-dioxa-8-octaneamine hydrochloride (814 mg, 1 mmol) was added to the reaction mixture and stirred 10 min. under argon at room temperature. The solution was diluted with EtOAc (50 mL), and washed with water 2×50 mL. The organic layer was dried over MgSO₄. Removal of the volatiles in vacuo provided pale yellow oil, which was purified by silica gel chromatography with MeOH—CH₂Cl₂ (0-20%). Yield: 610 mg, 50%, ¹H NMR (d₆-DMSO, 400 MHz): δ 8.20 (bs, 2H, —NH), 7.89 (d, J=7.6 Hz, 4H, —Ar), 7.84 (d, J=7.6 Hz, 4H, —Ar), 7.41 (t, J=6.8 Hz, 4H, —Ar), 7.35 (t, J=6.8 Hz, 4H, —Ar), 6.44 (bs, 2H, —NH), 3.6-3.4 (m, 6H, —CH₂O—, —CHAr—), 2.72-2.58 (m, 48H, —CH₂—), 1.40 (s, 18H, —CH₃). ¹³C NMR (d₆-DMSO, 100 MHz): δ 170.7, 170.3, 139.5, 137.5, 128.9, 127.3, 121.3, 120.0, 109.7, 80.12, 80.05, 69.5, 69.2, 59.8, 58.3, 55.6, 53.8, 53.2, 52.0, 51.7, 38.7, 38.3, 31.3, 27.9. ESI-MS calcd. for C₅₆H₉₂N₈O₁₄: 1221.5. found: [M+H]⁺=1221.8.

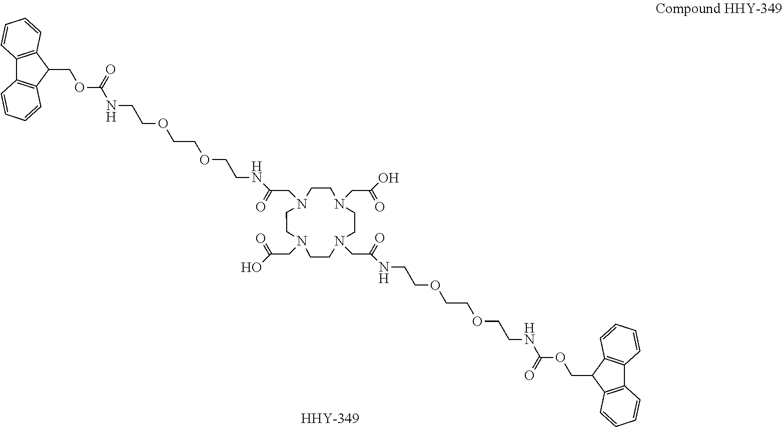 [SMILES: O=C(O)CN1CCN(CC(=O)NCCOCCOCCNC(=O)OCC2c3ccccc3-c3ccccc32)CCN(CC(=O)O)CCN(CC(=O)NCCOCCOCCNC(=O)OCC2c3ccccc3-c3ccccc32)CC1]

For removal of the tBu-group, compound HHY-348 (610 mg, 0.5 mmol) was dissolved in 10 mL 95% TFA/H₂O and the reaction mixture was stirred for 2 hours under argon at room temperature. The reaction was monitored by LC-MS. After the reaction was complete, the solvent was 3 times co-evaporated with toluene. The crude product was used without further purification in the next step. ESI-MS calcd. for C₅₈H₇₆N₈O₁₄: 1109.3. found: [M+H]⁺=1110.65.

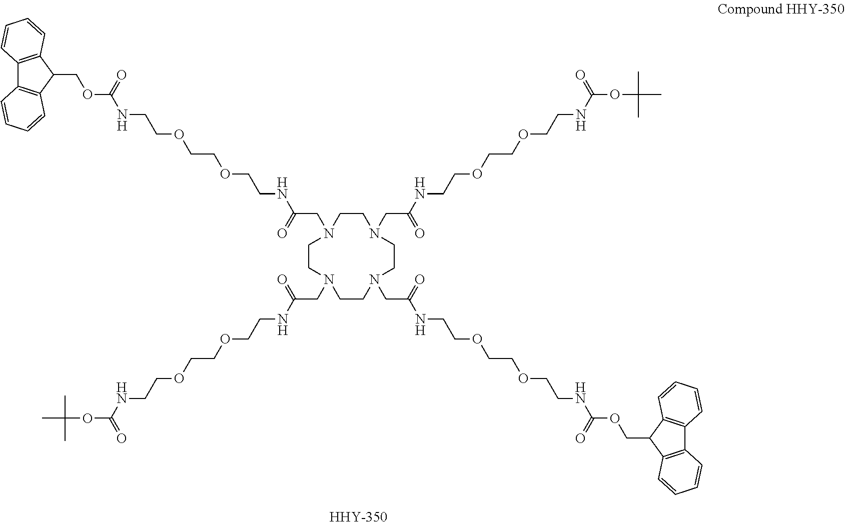 [SMILES: CC(C)(C)OC(=O)NCCOCCOCCNC(=O)CN1CCN(CC(=O)NCCOCCOCCNC(=O)OCC2c3ccccc3-c3ccccc32)CCN(CC(=O)NCCOCCOCCNC(=O)OC(C)(C)C)CCN(CC(=O)NCCOCCOCCNC(=O)OCC2c3ccccc3-c3ccccc32)CC1]

1 equiv. compound HHY-349 (0.5 mmol), 3 equiv. HATU (570 mg, 1.5 mmol) and 12 equiv. DIPEA (1.05 mL, 6 mmol) were dissolved in 30 mL DMF. After 10 min, 3 equiv. Boc-1-amino-3,6-dioxa-8-octanediamine (373 mg, 1.5 mmol) was added to the reaction mixture and stirred 30 min. under argon at room temperature. The reaction was monitored by LC-MS. After the reaction was complete, the reaction solution was directly purified by HPLC to give compound HHY-350 (565 mg, yield: 72%) as a white powder. ¹H NMR (d₆-DMSO, 400 MHz): δ 8.29 (bs, 2H, —NH), 7.94 (bs, 2H, —NH), 7.89 (d, J=7.6 Hz, 4H, —Ar), 7.68 (d, J=7.6 Hz, 4H, —Ar), 7.42 (t, J=6.8 Hz, 4H, —Ar), 7.32 (t, J=6.8 Hz, 4H, —Ar), 6.73 (bs, 2H, —NH), 6.27 (bs, 2H, —NH), 3.51-2.50 (m, 78H, —CH₂O—, —CHAr—, —CH₂—), 1.37 (bs, 18H, —CH₃). ¹³C NMR (d₆-DMSO, 100 MHz): δ 142.5, 139.4, 137.5, 128.9, 127.3, 109.7, 77.7, 69.6, 69.5, 69.4, 69.3, 69.2, 68.9, 67.5, 66.6, 57.8, 52.8, 38.3, 38.2, 28.2. ESI-MS calcd. for C₈₀H₁₂₀N₁₂O₂₀: 1569.9. found: [M+2H]²⁺=785.7.

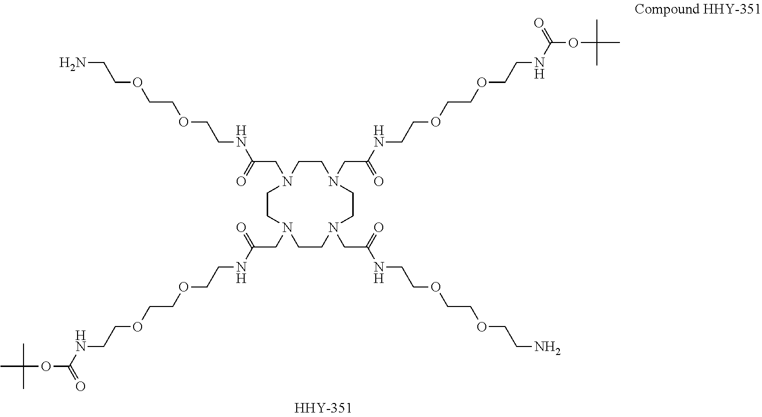 [SMILES: CC(C)(C)OC(=O)NCCOCCOCCNC(=O)CN1CCN(CC(=O)NCCOCCOCCN)CCN(CC(=O)NCCOCCOCCNC(=O)OC(C)(C)C)CCN(CC(=O)NCCOCCOCCN)CC1]

For removal of the Fmoc-group compound HHY-350 (471 mg, 0.3 mmol) was dissolved in 2 mL Et₂NH/DMF (1/4) and the reaction mixture was stirred for 30 min. at room temperature. The reaction was monitored by LC-MS. After the reaction was complete, the solvent was removed under reduced pressure. The crude product was used without further purification in the next step. ESI-MS calcd. for C₅₀H₁₀₀N₁₂O₁₆: 1125.4. found: [M+2H]²⁺=563.4.

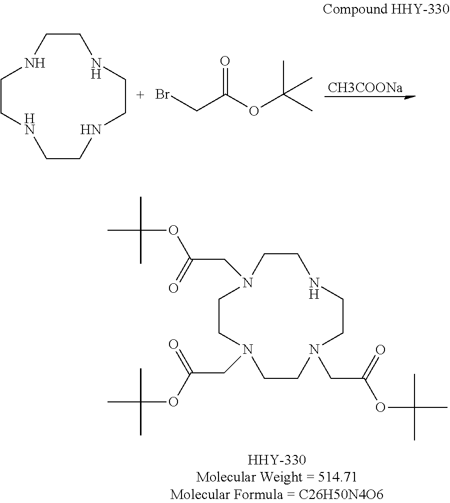

### 1,4,7-Tris(tert-butoxycarbonylmethyl)-1,4,7,10-tetraazacyclododecane hydrobromide (HHY-330)

To a suspension of cyclen (5.00 g, 29 mmol) and sodium acetate (7.86 g, 96 mmol) in N,N-dimethylacetamide (DMA, 60 mL) at −20° C. was added a solution of t-butyl bromoacetate (18.7 g, 14.1 mL, 96 mmol) in DMA (20 mL) dropwise over a period of 0.5 h. The temperature was maintained at −20° C. during the addition, after which the reaction mixture was allowed to come to room temperature. After 24 h of vigorous stirring, the reaction mixture was poured into water (300 mL) to give a clear solution. Solid KHCO₃ (15 g, 150 mmol) was added portion wise, and 4 precipitated as a white solid. The precipitate was collected by filtration and dissolved in CHCl₃ (250 mL). The solution was washed with water (100 mL), dried (MgSO4), filtered, and concentrated to about 20-30 mL. Ether (250 mL) was added, after which HHY-330 crystallized as a white fluffy solid. Yield: 12.5 g (73%). ESI-MS found: [M+H]⁺=515.5 (Moore, D. A. Org. Synth. 2008, 85, 10-14).

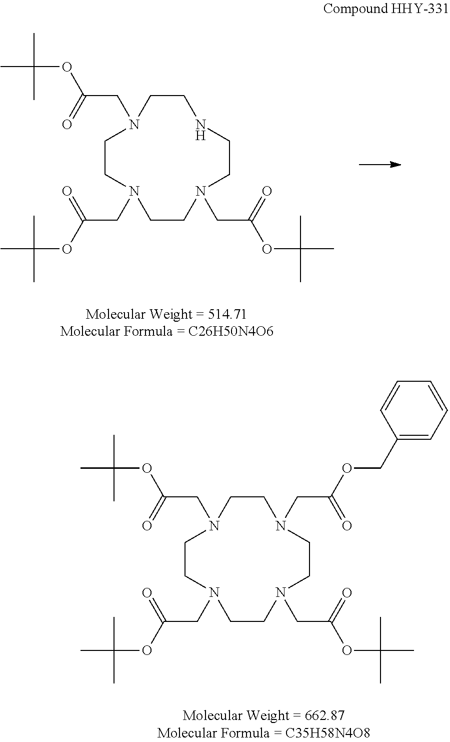

### Tri-tert-butyl 2,2′,2″-(10-(2-(benzyloxy)-2-oxoethyl)-1,4,7,10-tetraazacyclododecane-1,4,7-triyl)triacetate (HHY-331)

To a suspension of HHY-330 (12.5 g, 24 mmol) in acetonitrile, K₂CO₃ powder (5.0 g, 36 mmol, 1.5 eq.) and subsequently benzyl bromoacetate (5.6 g, 28.8 mmol, 1.2 eq.) were added. Reaction was stirred at room temperature for 3 hrs. Reaction process was monitored by TLC (CHCl₃-EtOH (9:1), HHY-331: Rf=0.8). The precipitated solids were removed by filtration and the filtrate was concentrated to give the crude product, which was purified by silica gel column chromatography using CH₂Cl₂-MeOH (100:0->90:10) to give colorless solids HHY-331 (Yield: 12.0 g, 75%). ESI-MS found: [M+H]⁺=663.5 (Strauch, R. C.; Mastarone, D. J.; Sukerkar, P. A.; Song, Y.; Ipsaro, J. J.; Meade, T. J. J. Am. Chem. Soc. 2011, 133, 16346-16349).

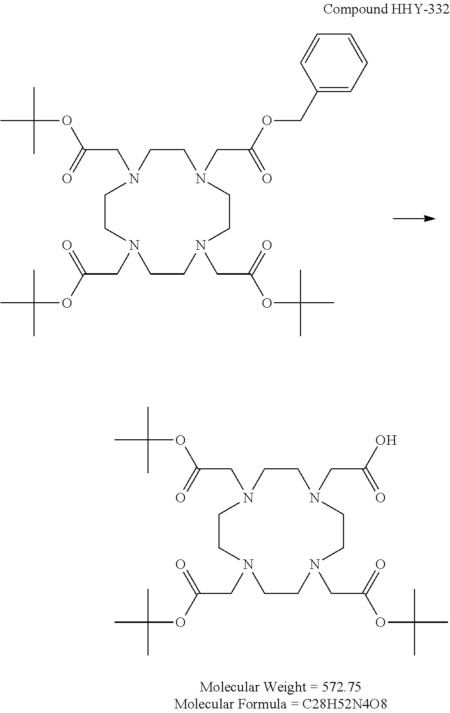 [SMILES: CC(C)(C)OC(=O)CN1CCN(CC(=O)OCc2ccccc2)CCN(CC(=O)OC(C)(C)C)CCN(CC(=O)OC(C)(C)C)CC1 ; CC(C)(C)OC(=O)CN1CCN(CC(=O)O)CCN(CC(=O)OC(C)(C)C)CCN(CC(=O)OC(C)(C)C)CC1]

### (4,7,10-Tris-tert-butoxycarbonylmethyl-1,4,7,10tetraaza-cyclododec-1-yl)-acetic acid

HHY-331 (1.2 g, 18 mmol) was hydrogenolyzed over 10% Pd on carbon (180 mg) in MeOH (100 mL) for 12 h. The Pd/C was removed by filtration and the MeOH removed by evaporation. Compounds were analyzed by LC MS and used without further purification. ESI-MS found: [M+H]⁺=573.4.

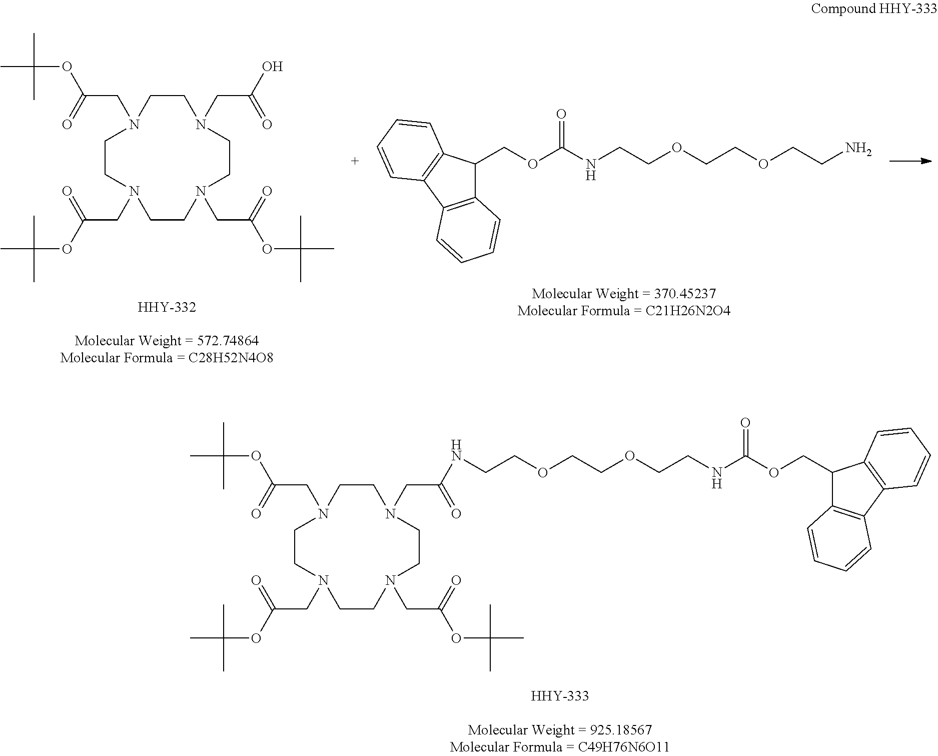 [SMILES: CC(C)(C)OC(=O)CN1CCN(CC(=O)O)CCN(CC(=O)OC(C)(C)C)CCN(CC(=O)OC(C)(C)C)CC1 ; NCCOCCOCCNC(=O)OCC1c2ccccc2-c2ccccc21 ; CC(C)(C)OC(=O)CN1CCN(CC(=O)NCCOCCOCCNC(=O)OCC2c3ccccc3-c3ccccc32)CCN(CC(=O)OC(C)(C)C)CCN(CC(=O)OC(C)(C)C)CC1]

1 equiv. HHY-332 (572 mg, 1 mmol), 1 equiv. HATU (380 mg, 1 mmol) and 4 equiv. DIPEA (0.7 ml, 4 mmol) were dissolved in 10 ml DMF. After 10 min, 1 equiv. 1-(9-Fluorenylmethyloxycarbonyl-amino)-3,6-dioxa-8-octaneamine hydrochloride (407 mg, 1 mmol) was added to the reaction mixture and stirred 10 min. under argon at room temperature. The solution was diluted with EtOAc (50 mL), and washed with water 2×50 mL. The organic layer was dried over MgSO₄. Removal of the volatiles in vacuo provided pale yellow oil, which was purified by silica gel chromatography with MeOH—CH₂Cl₂ (0-10%). Yield: 570 mg, 62%, ESI-MS found: [M+H]⁺=926.7

¹H NMR (d₆-DMSO, 400 MHz): δ 8.20 (t, J=4.8 Hz, 1H, —NH), 7.89 (d, J=7.6 Hz, 2H, —Ar), 7.68 (d, J=7.6 Hz, 2H, —Ar), 7.41 (t, J=6.8 Hz, 2H, —Ar), 7.32 (t, J=6.8 Hz, 2H, —Ar), 7.29 (t, J=5.7 Hz, 1H, —NH), 4.29 (d, J=6.8 Hz, 2H, —CH₂O—), 4.20 (t, J=6.8 Hz, 1H, —CHAr—), 3.51-2.49 (m, 36H, —CH₂—), 1.43-1.41 (m, 27H, —CH₃).

¹³C NMR (d₆-DMSO, 100 MHz): δ 172.5, 171.6, 162.3, 143.9, 140.7, 127.6, 127.0, 125.1, 120.1, 81.1, 80.9, 69.4, 68.9, 40.2, 38.9, 38.2, 35.8, 30.8, 27.8, 27.6

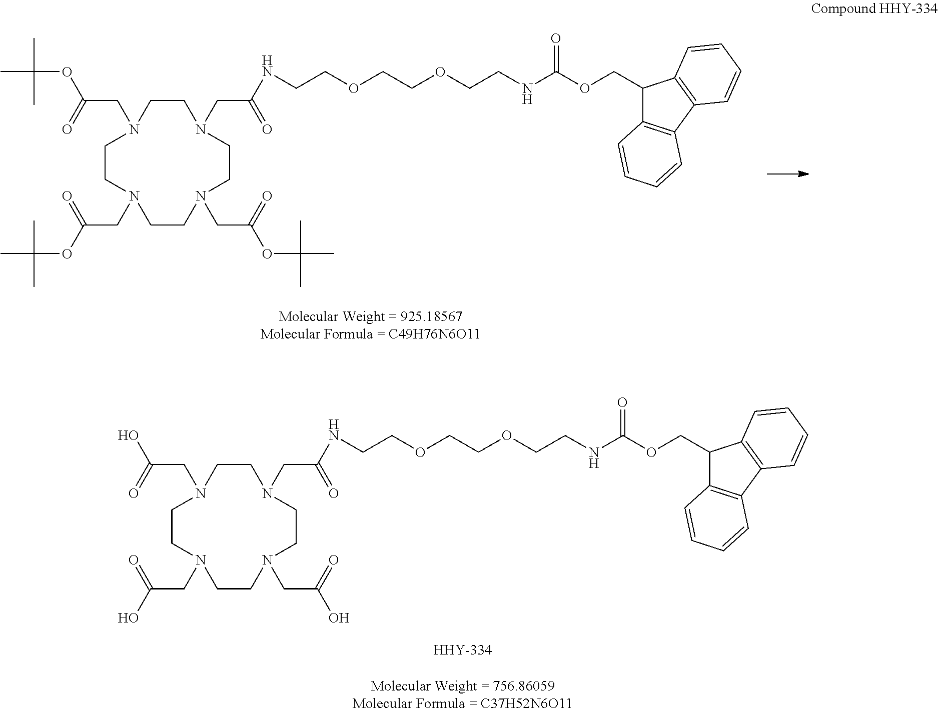 [SMILES: CC(C)(C)OC(=O)CN1CCN(CC(=O)NCCOCCOCCNC(=O)OCC2c3ccccc3-c3ccccc32)CCN(CC(=O)OC(C)(C)C)CCN(CC(=O)OC(C)(C)C)CC1 ; O=C(O)CN1CCN(CC(=O)O)CCN(CC(=O)NCCOCCOCCNC(=O)OCC2c3ccccc3-c3ccccc32)CCN(CC(=O)O)CC1]

For removal of the tBu-group, compound HHY-333 (925 mg, 1 mmol) was dissolved in 10 ml 95% TFA/H₂O and the reaction mixture was stirred for 2 hours under argon at room temperature. The reaction was monitored by LC-MS. After the reaction was complete, the solvent was 3 times co-evaporated with toluene. The crude product was used without further purification in the next step. ESI-MS found: [M+H]⁺=758.4.

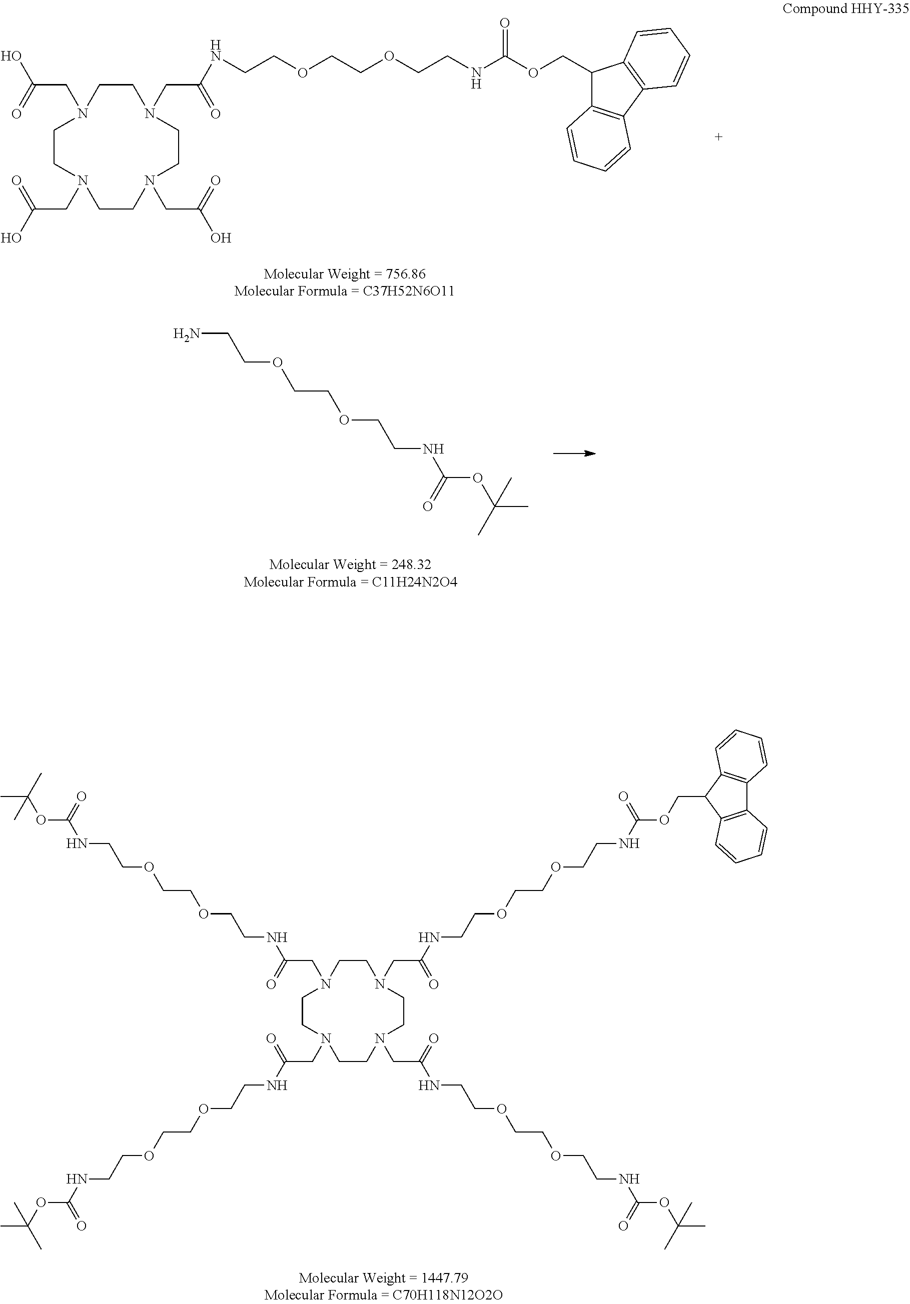 [SMILES: O=C(O)CN1CCN(CC(=O)O)CCN(CC(=O)NCCOCCOCCNC(=O)OCC2c3ccccc3-c3ccccc32)CCN(CC(=O)O)CC1 ; CC(C)(C)OC(=O)NCCOCCOCCN ; CC(C)(C)OC(=O)NCCOCCOCCNC(=O)CN1CCN(CC(=O)NCCOCCOCCNC(=O)OCC2c3ccccc3-c3ccccc32)CCN(CC(=O)NCCOCCOCCNC(=O)OC(C)(C)C)CCN(CC(=O)NCCOCCOCCNC(=O)OC(C)(C)C)CC1]

1 equiv. HHY-334 (1 mmol), 3 equiv. HATU (1140 mg, 3 mmol) and 12 equiv. DIPEA (2100 μl, 12 mmol) were dissolved in 30 ml DMF. After 10 min, 3 equiv. Boc-1-amino-3,6-dioxa-8-octanediamine (745 mg, 3 mmol) was added to the reaction mixture and stirred 30 min. under argon at room temperature. The reaction was monitored by LC-MS. After the reaction was complete, the reaction solution was directly purified by HPLC to give HHY-335 (800 mg, yield: 55%) as a white powder.

¹H NMR (d₆-DMSO, 400 MHz): δ 8.34 (bs, 3H, —NH), 7.94 (bs, 1H, —NH), 7.89 (d, J=7.6 Hz, 2H, —Ar), 7.68 (d, J=7.6 Hz, 2H, —Ar), 7.42 (t, J=6.8 Hz, 2H, —Ar), 7.32 (t, =6.8 Hz, 2H, —Ar), 7.29 (t, J=5.1 Hz, 1H, —NH), 6.74 (t, J=4.78 Hz, 3H, —NH), 4.29 (d, J=6.8 Hz, 2H, —CH₂O—), 4.21 (t, J=6.8 Hz, 1H, —CHAr—), 3.62-3.04 (m, 72H, —CH₂—), 1.37 (bs, 27H, —CH₃).

¹³C NMR (d₆-DMSO, 100 MHz): δ 158.12, 158.10, 157.82, 158.78, 156.1, 155.6, 143.9, 140.7, 127.6, 127.0, 125.1, 120.9, 118.0, 115.1 77.6, 69.52, 69.47, 69.4, 69.2, 69.1, 68.8, 65.3, 54.5, 49.6, 46.7, 40.2, 40.0, 38.9, 28.2.

ESI-MS calcd. for C₇₀H₁₁₈N₁₂O₂₀: 1447.79. found: [M+2H]²⁺=724.6.

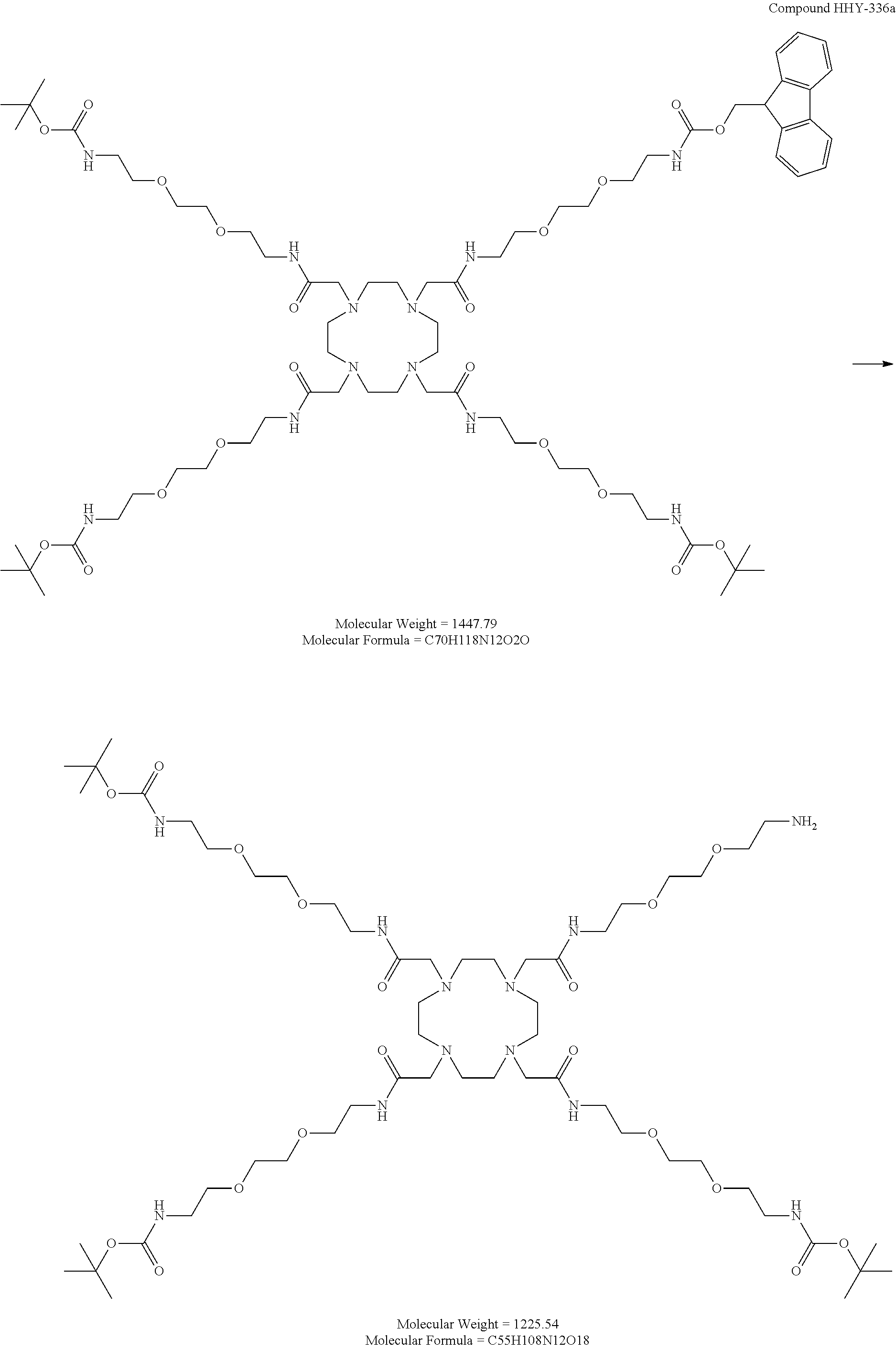 [SMILES: CC(C)(C)OC(=O)NCCOCCOCCNC(=O)CN1CCN(CC(=O)NCCOCCOCCNC(=O)OCC2c3ccccc3-c3ccccc32)CCN(CC(=O)NCCOCCOCCNC(=O)OC(C)(C)C)CCN(CC(=O)NCCOCCOCCNC(=O)OC(C)(C)C)CC1 ; CC(C)(C)OC(=O)NCCOCCOCCNC(=O)CN1CCN(CC(=O)NCCOCCOCCN)CCN(CC(=O)NCCOCCOCCNC(=O)OC(C)(C)C)CCN(CC(=O)NCCOCCOCCNC(=O)OC(C)(C)C)CC1]

For removal of the Fmoc-group compound HHY-335 (724 mg, 0.5 mmol) was dissolved in 2 ml Et₂NH/DMF (1/4) and the reaction mixture was stirred for 30 min. at room temperature. The reaction was monitored by LC-MS. After the reaction was complete, the solvent was removed under reduced pressure. The crude product was used without further purification in the next step. ESI-MS found: [M+2H]²⁺=613.5

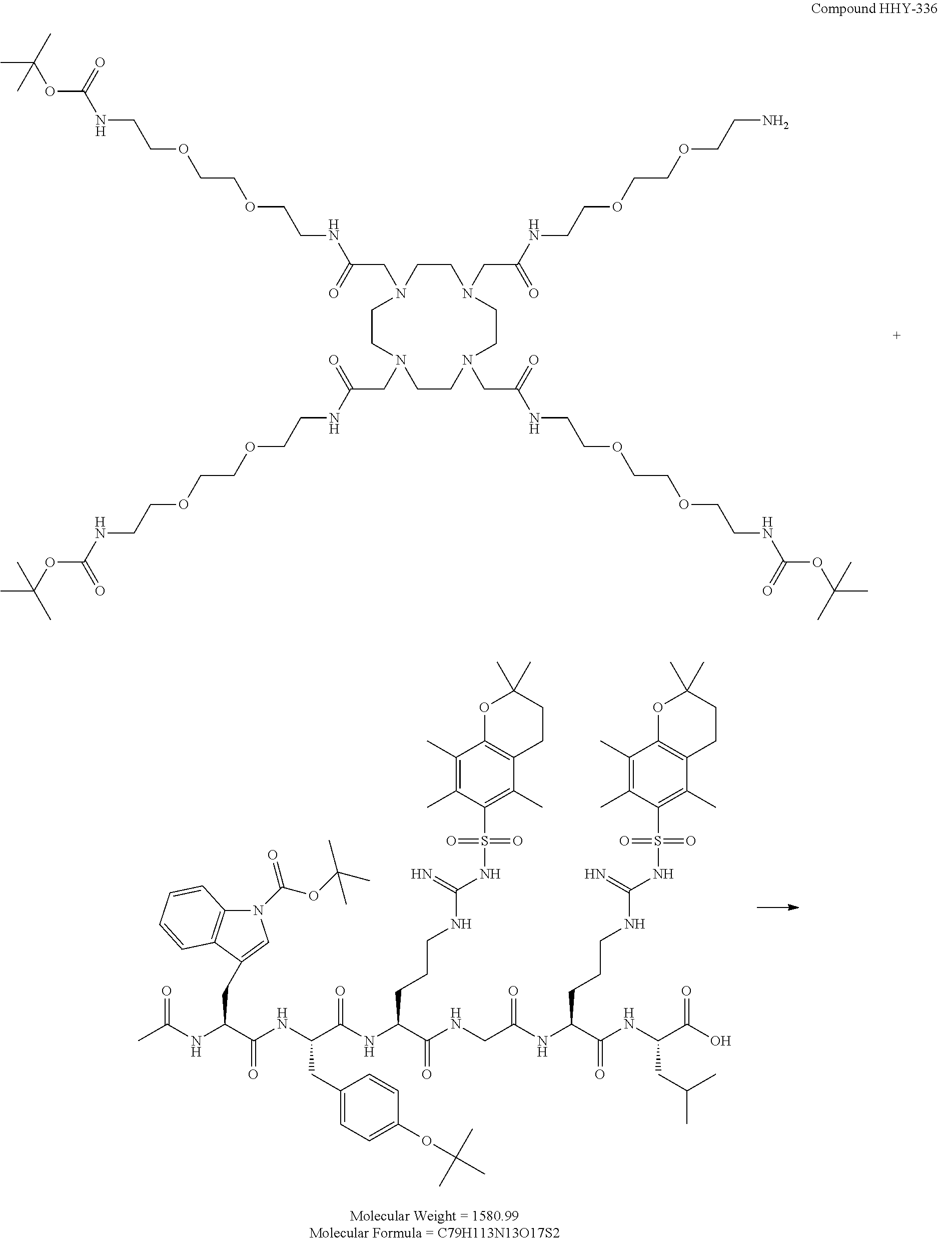

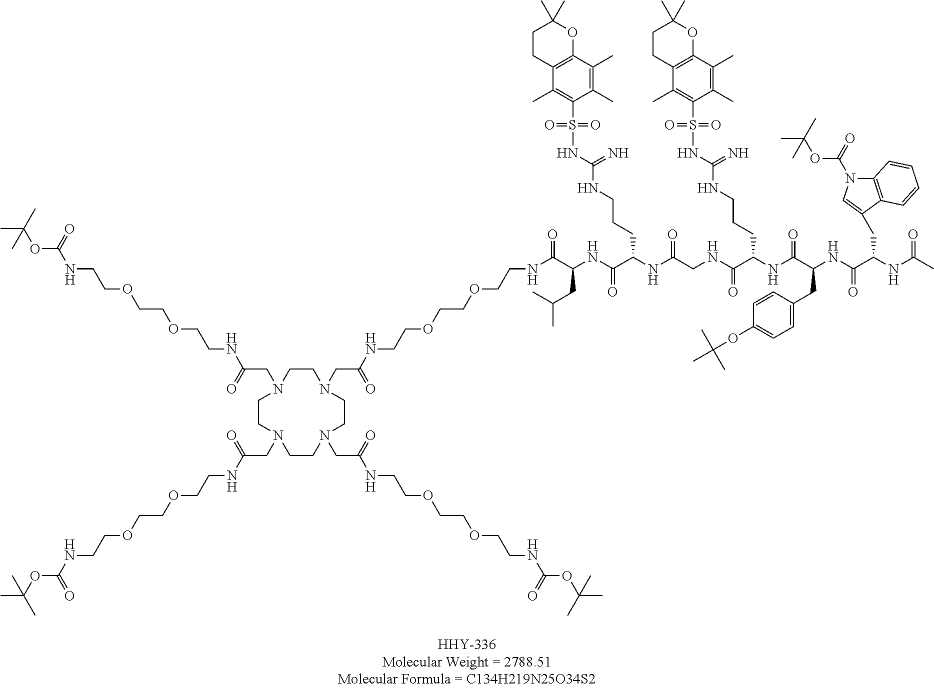 [SMILES: CC(=O)N[C@@H](Cc1cn(C(=O)OC(C)(C)C)c2ccccc12)C(=O)N[C@@H](Cc1ccc(OC(C)(C)C)cc1)C(=O)N[C@@H](CCCNC(=N)NS(=O)(=O)c1c(C)c(C)c2c(c1C)CCC(C)(C)O2)C(=O)NCC(=O)N[C@@H](CCCNC(=N)NS(=O)(=O)c1c(C)c(C)c2c(c1C)CCC(C)(C)O2)C(=O)N[C@@H](CC(C)C)C(=O)NCCOCCOCCNC(=O)CN1CCN(CC(=O)NCCOCCOCCNC(=O)OC(C)(C)C)CCN(CC(=O)NCCOCCOCCNC(=O)OC(C)(C)C)CCN(CC(=O)NCCOCCOCCNC(=O)OC(C)(C)C)CC1]

1 equiv. AcWYRGRL (295 mg, 0.25 mmol), 1 equiv. HATU (95 mg, 0.25 mmol) and 4 equiv. 2, 4, 6-collidine (132 μl, 1 mmol) were dissolved in 20 ml DMF. After 10 min, 1 equiv. 1 equiv. HHY-336a (306 mg, 0.25 mmol) was added to the reaction mixture and stirred 30 min. under argon at room temperature. The reaction solution was directly purified by HPLC to give HHY-336 (400 mg, yield: 57%) as a white powder. ESI-MS found: [M+3H]³⁺=930.5

ESI-HRMS calcd. for C₁₃₄H₂₁₉N25O₃₄S₂: 2786.56174. found: 2786.56164 ([M+2H]²⁺=1394.28810).

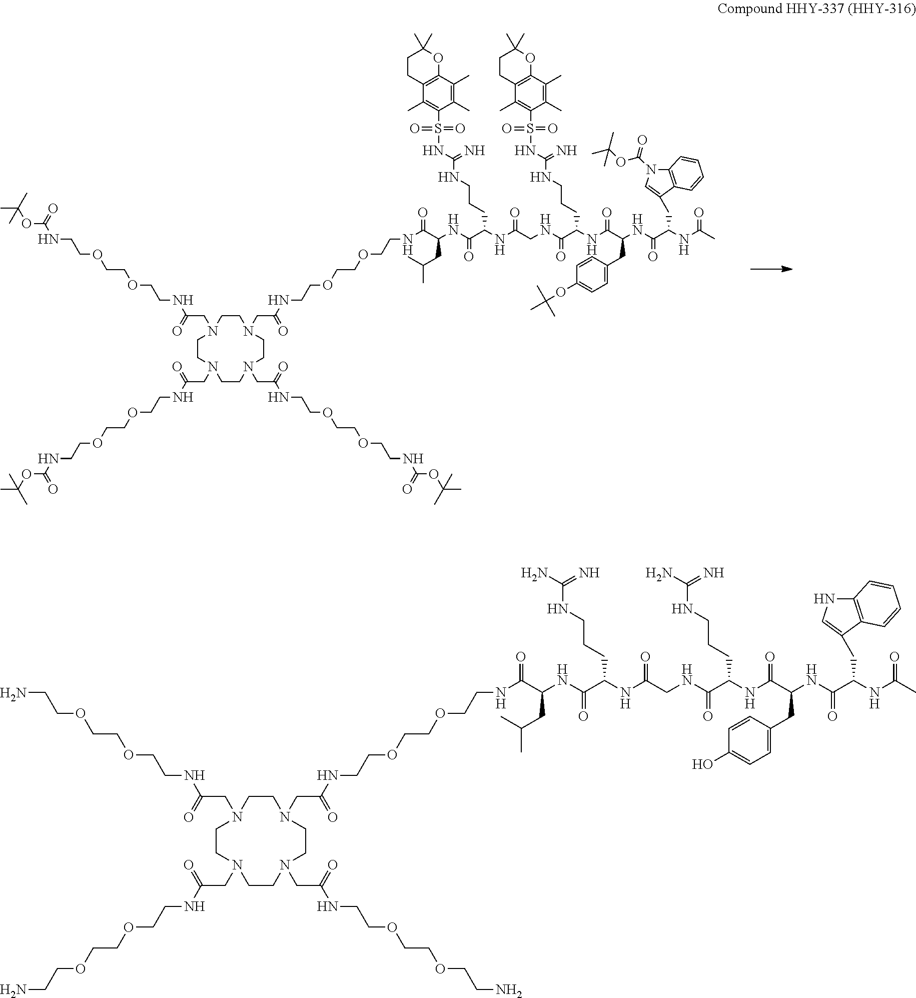

For removal of all of the protection groups, the crude HHY-336 was dissolved in 10 ml 95% TFA/H₂O and the reaction mixture was stirred for 3 hours under argon at room temperature. The reaction was monitored by LC-MS. After the reaction was complete, the solvent was 3 times co-evaporated with toluene. The crude product was purified by HPLC to give HHY-337 (yield: 65%) as a white powder.

ESI-MS found: [M+2H]²⁺=900.3

HRMS calcd. for C₈₂H₁₄₃N₂₅O₂₀: 1798.09412. found: 1798.09428 ([M+4H]⁴⁺=450.53085).

Synthesis of Pepstatine A control probe HHY325

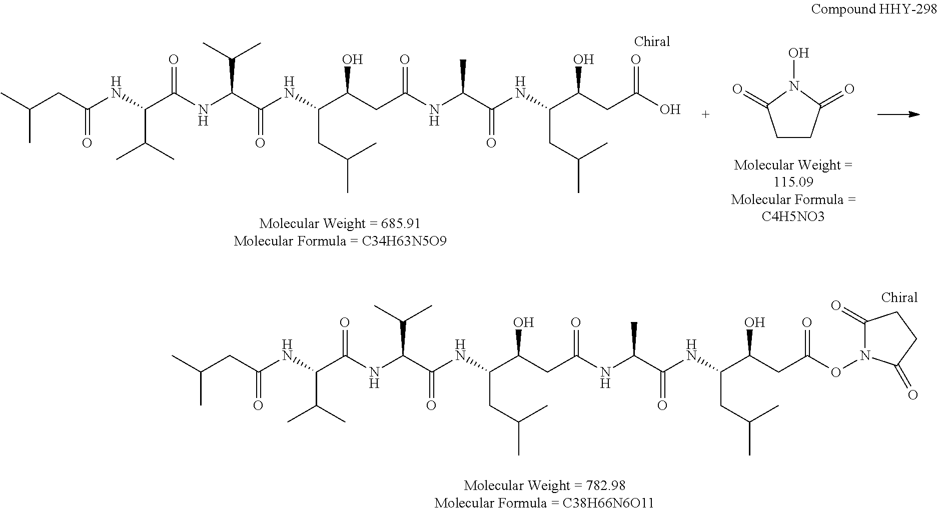 [SMILES: CC(C)CC(=O)N[C@H](C(=O)N[C@H](C(=O)N[C@@H](CC(C)C)[C@@H](O)CC(=O)N[C@@H](C)C(=O)N[C@@H](CC(C)C)[C@@H](O)CC(=O)O)C(C)C)C(C)C ; O=C1CCC(=O)N1O ; CC(C)CC(=O)N[C@H](C(=O)N[C@H](C(=O)N[C@@H](CC(C)C)[C@@H](O)CC(=O)N[C@@H](C)C(=O)N[C@@H](CC(C)C)[C@@H](O)CC(=O)ON1C(=O)CCC1=O)C(C)C)C(C)C]

Pepstatin A (20 mg, 30 μmol), EDCI (58 mg, 0.3 mmol, 10 eq.) and NHS (35 mg, 0.3 mmol, 10 eq.) were placed in a flask. To this was added anhydrous DMF (2 ml) and the reaction stirred overnight. The DMF was removed in high vacuo. The solid was loosened from the vessel walls, washed with water (10 ml) and collected by filtration. The solid was further washed with water (50 ml) and then with diethyl ether (5 ml). The wet solid was then dried for 24 h under vacuum over anhydrous P₂O₅ (Org. Biomol. Chem., 2006, 4, 1817-1830).

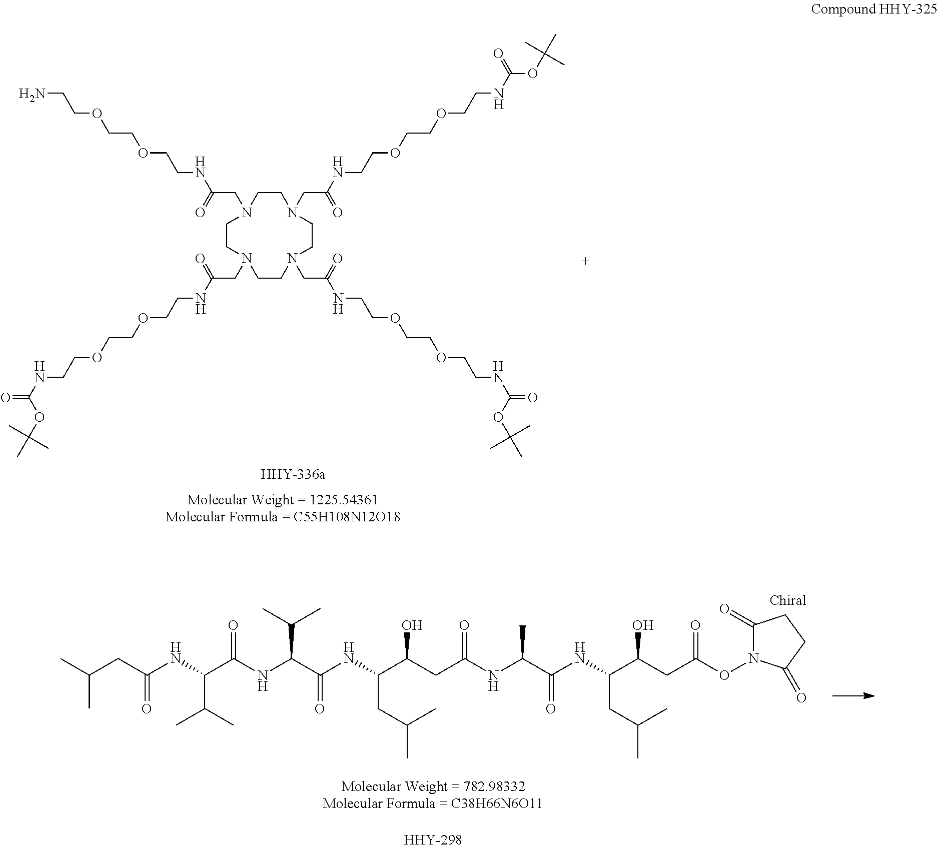

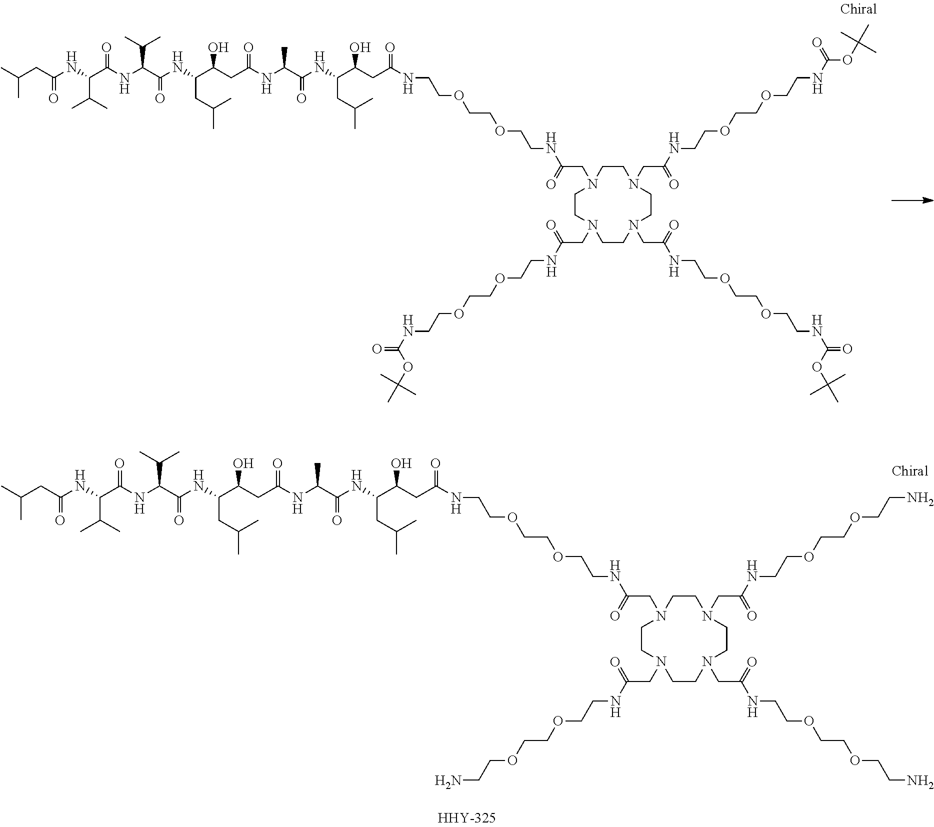

HHY-336a (12 mg, 10 plop was dissolved in 1.5 ml DMF, followed by the addition of 1 equiv. pepstatin A NHS ester HHY-298 (8 mg, 10 μmol) and 5 equiv. DIPEA (10 μl, 50 gimp. The reaction mixture was stirred under argon at room temperature for 24 h. After the reaction was complete, the solvent was removed by evaporation and the residue was dissolved in 10 ml 50% TFA/CH₂Cl₂. Removal of Boc group was monitored by LC-MS. After 30 min, the solvent was co-evaporated with toluene and the crude product was directly purified by HPLC to give to give HHY-325 (6 mg, Yield 38%) as a white powder.

ESI-MS found: [M+2H]²⁺=797.3.

HRMS calcd. for C₇₄H₁₄₅N₁₇O₂₀: 1592.08518. found: 1592.08514 ([M+2H]²⁺=797.04985).

### EXAMPLE 1

### Compound HHY-312

Targeting Peptide Fluorescent Conjugate AcWYRGRL-DOTA-Cy5.5

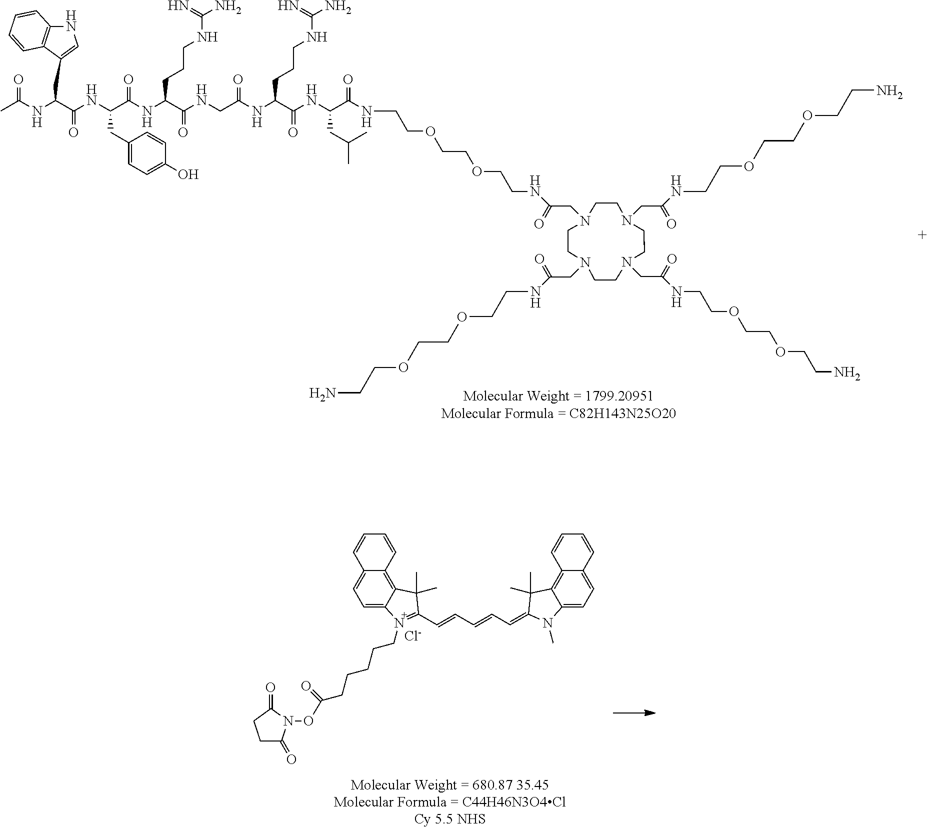

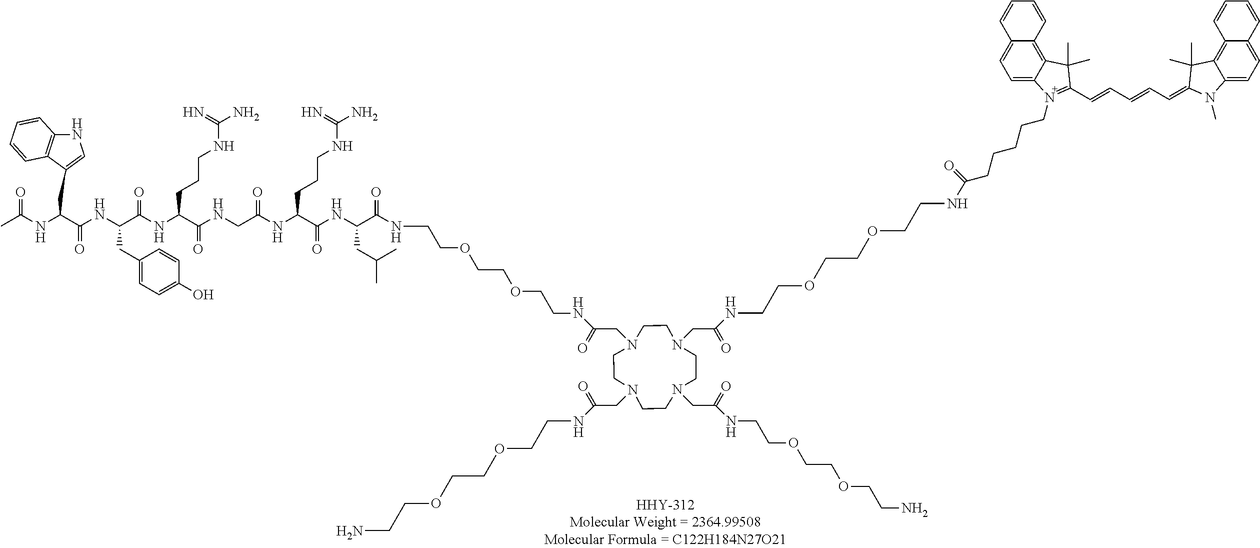

HHY-316 (18 mg, 10 μmol) was dissolved in 1.5 ml DMF, followed by the addition of 1 equiv. Cy5.5 NHS ester (7 mg, 10 μmol) and 5 equiv. DIPEA (10 μl, 50 μmol). The reaction mixture was stirred under argon at room temperature for 12 h. After the reaction was complete, the solvent was removed under reduced pressure. After the reaction was complete, the reaction solution was directly purified by HPLC to give to give HHY-312 (15 mg, Yield 63%) as a blue powder.

ESI-MS found: [M+2H]³⁺=788.9.

HRMS calcd. for C₁₂₂H₁₈₄N₂₇O₂₁: 2363.41543. found: 2363.41518 ([M+4H]⁵⁺=473.48886).

### EXAMPLE 2

### Compound HHY-316Gd

Targeting Peptide MRI Imaging Conjugate AcWYRGRL-DOTA-[Gd3+]

Gadolinium Complex Formation:

Complexes were prepared by adding a GdCl₃ stock solution to a DOTA-peptide ligand solution, in stoichiometric amounts (1:1). The pH was adjusted to 6 using 1 N—NaOH and stirred for 48 h. and then centrifuged to remove any precipitated Gd(OH)₃. The presence of free Gd³⁺ was evaluated by colorimetry using xylenol orange as an indicator. Resultant peptide complexes were further purified by HPLC. The purified complex solution was lyophilized to give the gadolinium complex as a powder solid.

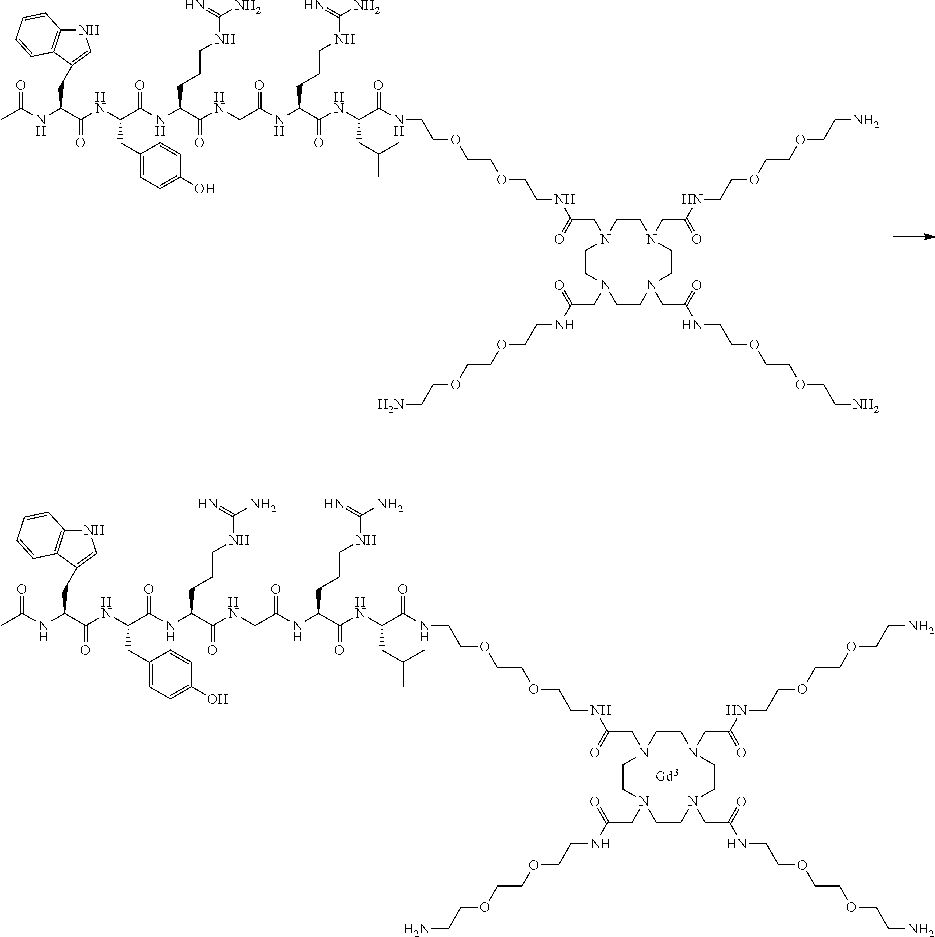

ESI-MS found: [M]³⁺=652.1.

HRMS calcd. for C₈₂H₁₄₃N₂₅O₂₀Gd: 1956.01656. found: 1956.01628 ([M+H]⁴⁺=489.25589).

### EXAMPLE 3

### Compound HHY-338

Targeting Peptide Bimodal Imaging Conjugate AcWYRGRL-DOTA-Cy5.5-[Gd3+]

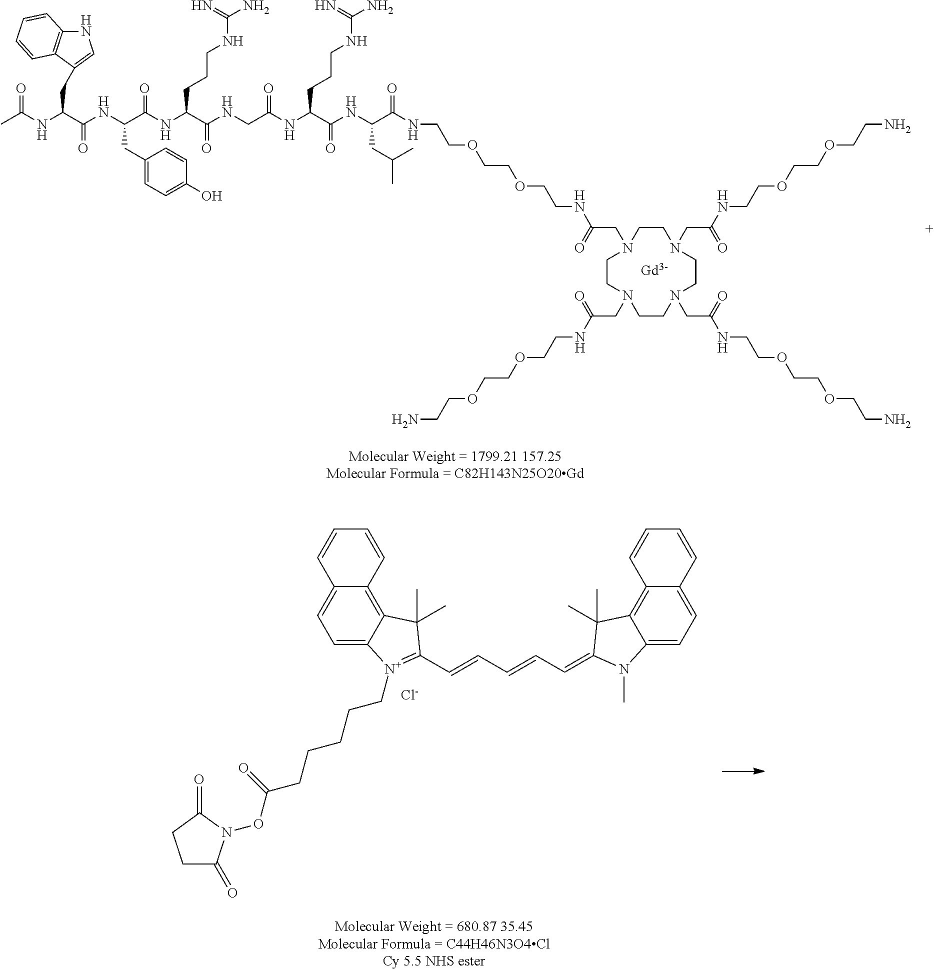

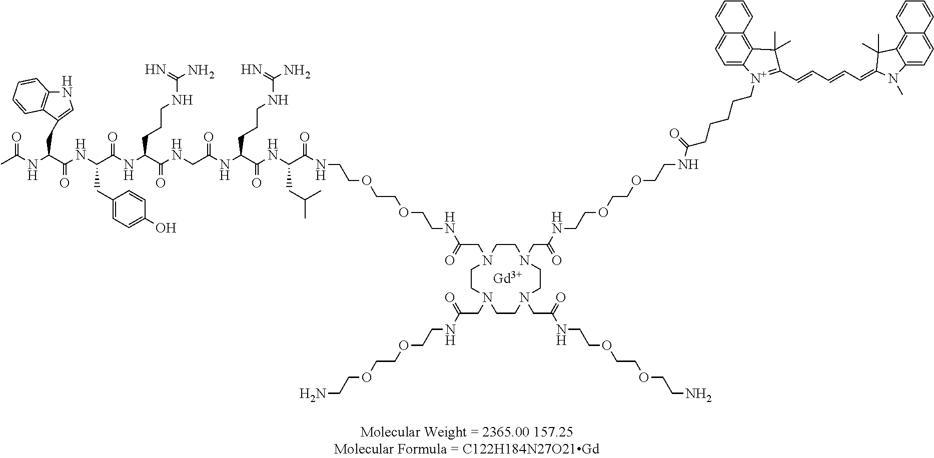

HHY-316Gd (19 mg, 10 μmol) was dissolved in 1.5 ml DMF, followed by the addition of 1 equiv. Cy5.5 NHS ester (7 mg, 10 μmol) and 5 equiv. DIPEA (10 μl, 50 μmol). The reaction mixture was stirred under argon at room temperature for 12 h. After the reaction was complete, the solvent was removed under reduced pressure. After the reaction was complete, the reaction solution was directly purified by HPLC to give to give HHY-338 (13 mg, Yield 51%) as a blue powder. ESI-MS found: [M]⁴⁺=630.2.

HRMS calcd. for C₁₂₂H₁₈₄N₂₈O₂₀Gd: 2521.33792. found: 2521.33802 ([M+H]⁴⁺=504.46906).

### EXAMPLE 4

### Compound HHY-327

Targeting Peptide Drug Conjugate AcWYRGRL-DOTA-Pepstatin A

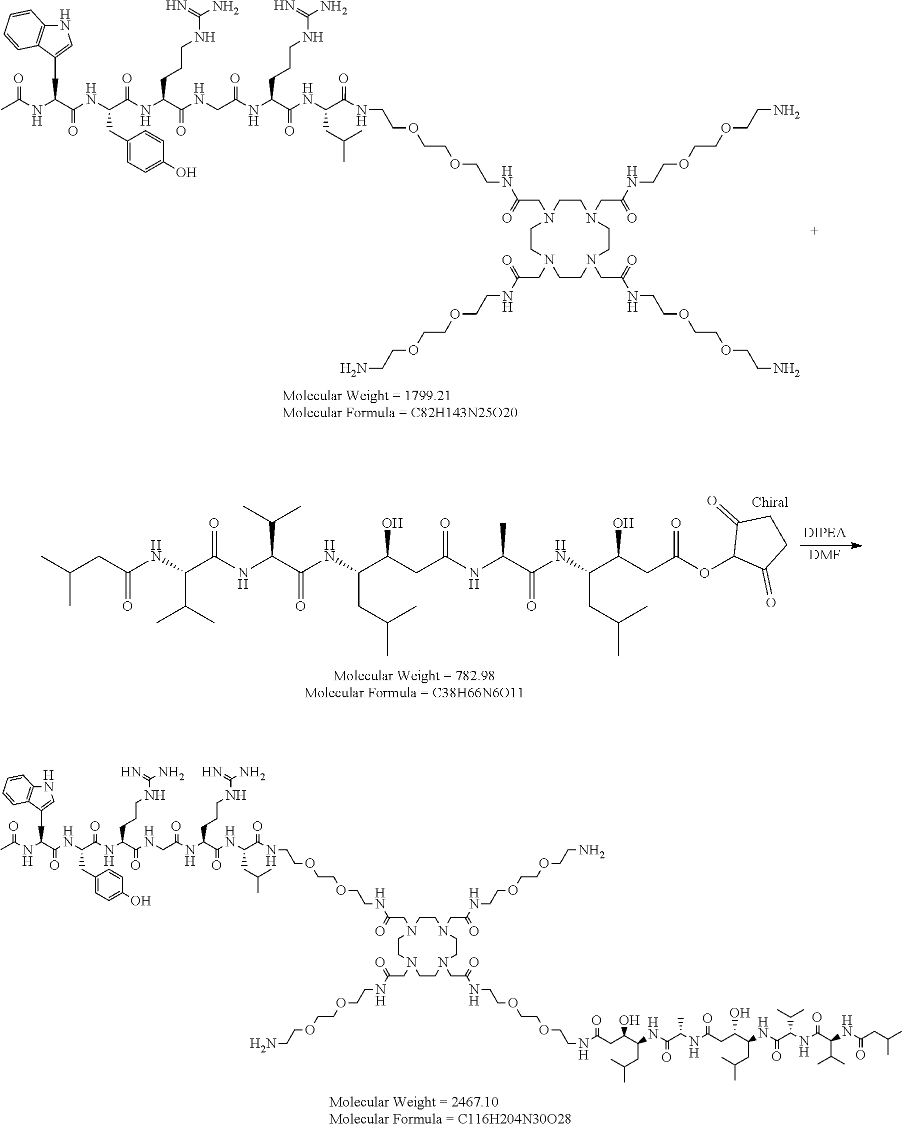

2 equiv. HHY-316 (36 mg, 20 μmol) was dissolved in 2 ml DMF, followed by the addition of 1 equiv. pepstatin A NHS ester HHY-298 (8 mg, 10 μmol) and 6 equiv. DIPEA (10 μl, 60 μmol). The reaction mixture was stirred under argon at room temperature for 2 days. The solvent was directly purified by preparative HPLC to give HHY-327 (5 mg, Yield 20%) as a white powder. ESI-MS found: [M+2H]²⁺=824.7.

ESI-MS calcd. for C₁₁₆H₂₀₄N₃₀O₂₈: 2465.5. found: [M+3H]³⁺=824.7.

### EXAMPLE 5

### Compound HHY328

Targeting Peptide Drug Conjugate AcWYRGRL-DOTA-(Pepstatin A)₃

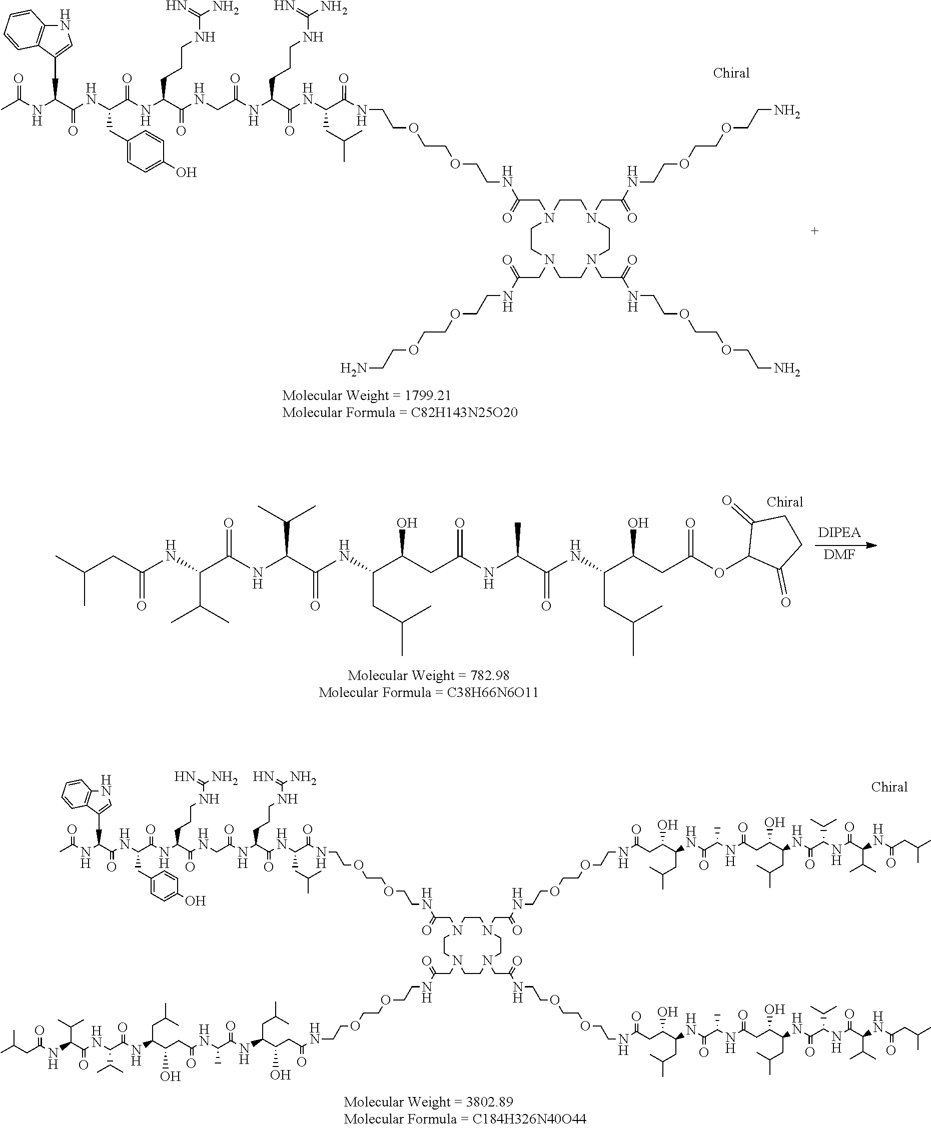

1 equiv. HHY-316 (9 mg, 5 μmol) was dissolved in 1.5 ml DMF, followed by the addition of 3 equiv. pepstatin A NHS ester HHY-298 (12 mg, 15 μmol) and 12 equiv. DIPEA (10 λl, 60 μmol). The reaction mixture was stirred under argon at room temperature for 2 days. The solvent was directly purified by preparative HPLC to give HHY-328 (4.3 mg, Yield: 22.6%) as a white powder. The product is only slightly soluble in DMSO after lyophilization.

ESI-MS calcd. for C₁₈₄H₃₂₆N₄₀O₄₄: 3802.9. found: [M+3H]³⁺=1268.5.

Synthesis of Scrambled Control of Example 1: Compound HHY-322 AcYRLGRW-DOTA-Cy5.5

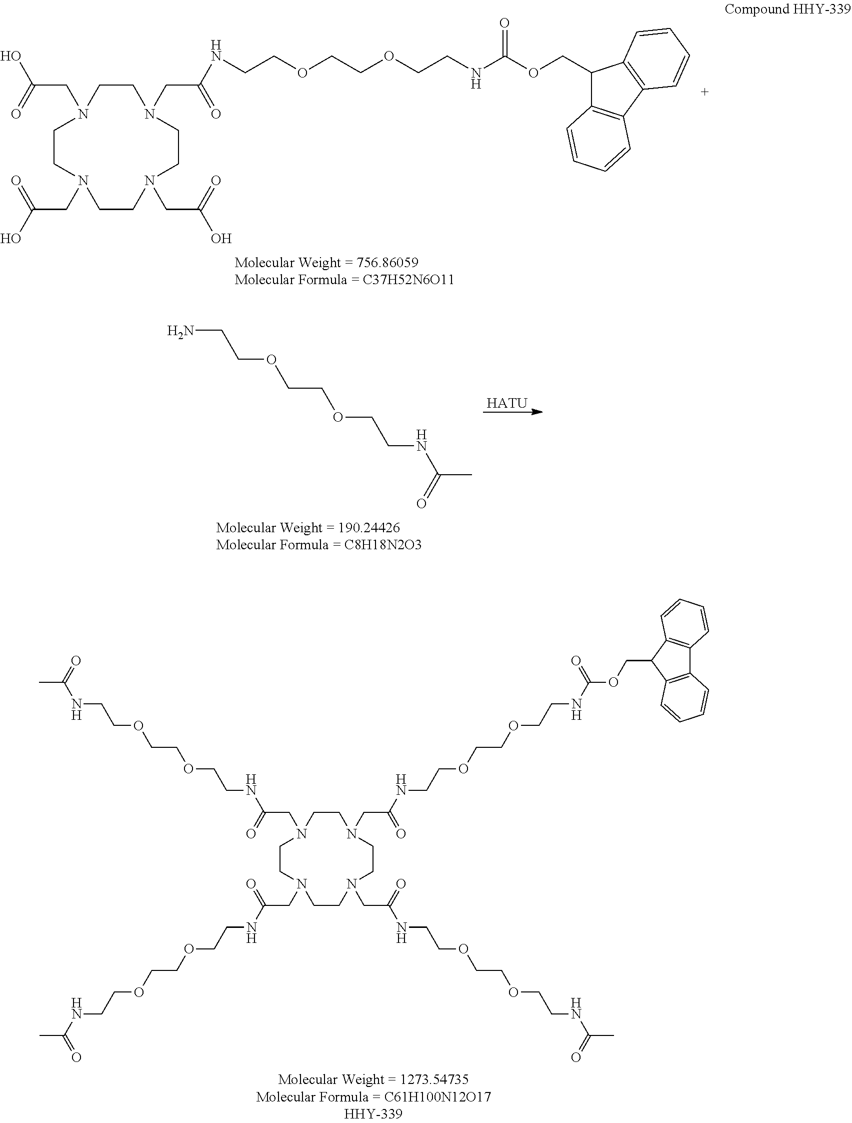 [SMILES: O=C(O)CN1CCN(CC(=O)O)CCN(CC(=O)NCCOCCOCCNC(=O)OCC2c3ccccc3-c3ccccc32)CCN(CC(=O)O)CC1 ; CC(=O)NCCOCCOCCN ; "HATU" ; CC(=O)NCCOCCOCCNC(=O)CN1CCN(CC(=O)NCCOCCOCCNC(C)=O)CCN(CC(=O)NCCOCCOCCNC(=O)OCC2c3ccccc3-c3ccccc32)CCN(CC(=O)NCCOCCOCCNC(C)=O)CC1]

1 equiv. HHY-334 (0.1 mmol), 3 equiv. HATU (114 mg, 0.3 mmol) and 12 equiv. DIPEA (210 μl, 1.2 mmol) were dissolved in 10 ml DMF. After 10 min, 3 equiv. N-[2-[2-[2-Amino-ethoxy]-ethoxy]-ethyl]-acetamide (57 mg, 0.3 mmol) was added to the reaction mixture and stirred 30 min. under argon at room temperature. The reaction was monitored by LC-MS. After the reaction was complete, the reaction solution was directly purified by HPLC to give HHY-339 (90 mg, yield: 70%) as a white powder. ESI-MS found: [M+H]⁺=1273.9.

¹H NMR (d₆-DMSO, 400 MHz): δ 8.32 (bs, 3H, −8.20, —NH), 7.90-7.85 (m, 6H, —Ar, —NH), 7.68 (d, J=7.2 Hz, 2H, —Ar), 7.42 (t, J=7.2 Hz, 2H, —Ar), 7.34-7.29 (m, 3H, —Ar, —NH), 4.29 (d, J=7.0 Hz, 2H, —CH₂O—), 4.21 (t, J=7.0 Hz, 1H, —CHAr—), 3.68-3.13 (m, 72H, —CH₂—), 1.80 (bs, 9H, —CH₃).

¹³C NMR (d₆-DMSO, 100 MHz): δ 127.6, 127.1, 125.1, 120.2, 99.8, 69.6, 69.5, 69.1, 68.8, 40.0, 39.8, 39.6, 38.7, 38.5, 22.6.

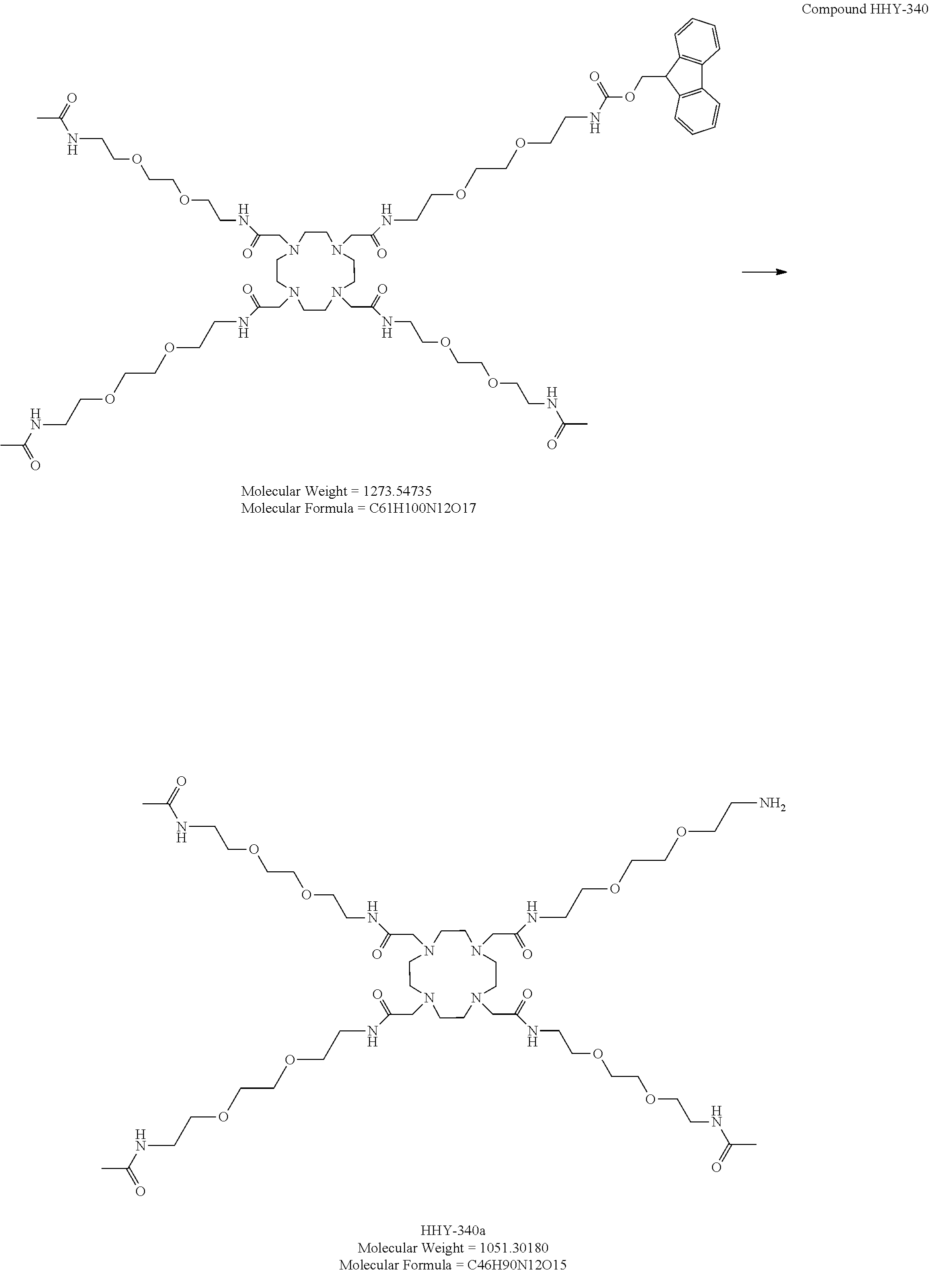 [SMILES: CC(=O)NCCOCCOCCNC(=O)CN1CCN(CC(=O)NCCOCCOCCNC(C)=O)CCN(CC(=O)NCCOCCOCCNC(=O)OCC2c3ccccc3-c3ccccc32)CCN(CC(=O)NCCOCCOCCNC(C)=O)CC1 ; CC(=O)NCCOCCOCCNC(=O)CN1CCN(CC(=O)NCCOCCOCCN)CCN(CC(=O)NCCOCCOCCNC(C)=O)CCN(CC(=O)NCCOCCOCCNC(C)=O)CC1]

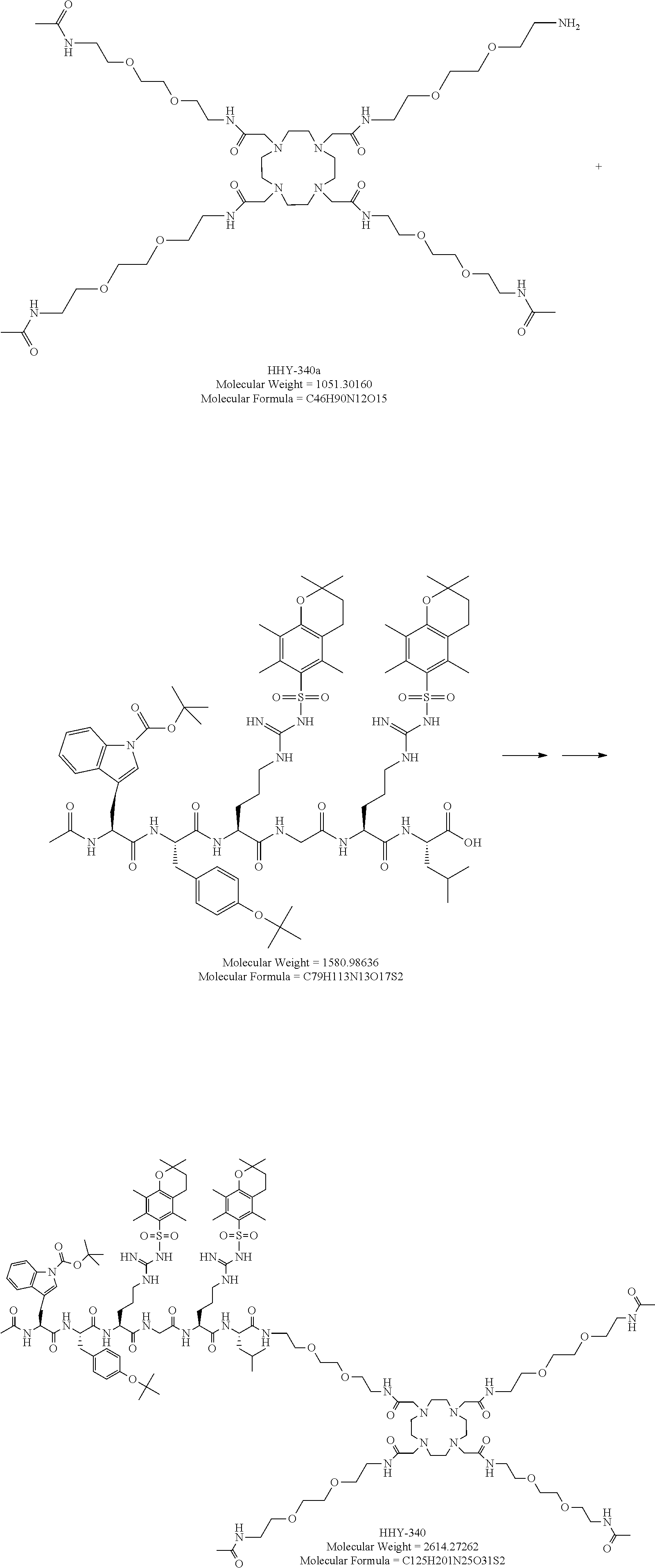

For removal of the Fmoc-group, HHY-339 (50 μmol) was dissolved in 2 ml Et₂NH/DMF (1/4) and the reaction mixture was stirred for 10 min. at room temperature. The solvent was removed under reduced pressure. The crude product HHY-340a was used without further purification in the next step.

ESI-MS calcd. for C₄₆H₉₀N₁₂O₁₅: 1051.3. found: [M+2H]²⁺=526.6.

Scrambled peptide sequence YRLGRW (79 mg, 50 μmol), 1 equiv. HATU (19 mg, 50 μmol) and 3 equiv. DIPEA (26 μl, 150 μmol) were solved in 2 ml DMF and stirred for 10 min. at room temperature. Subsequently 1 equiv. HHY-340a was added to the reaction mixture, which was stirred under argon at room temperature for 1 h. The reaction was monitored by LC-MS. After the reaction was complete, the reaction solution was directly purified by HPLC to give HHY-340 (81 mg, yield: 62%) as a white powder. ESI-MS found: [M+2H]²⁺=1308.1

HRMS calcd. for C₁₂₅H₂₀₁N₂₅O₃₁S₂: 2612.43616. found: 2612.43666 ([M+2H]²⁺=1307.22561).

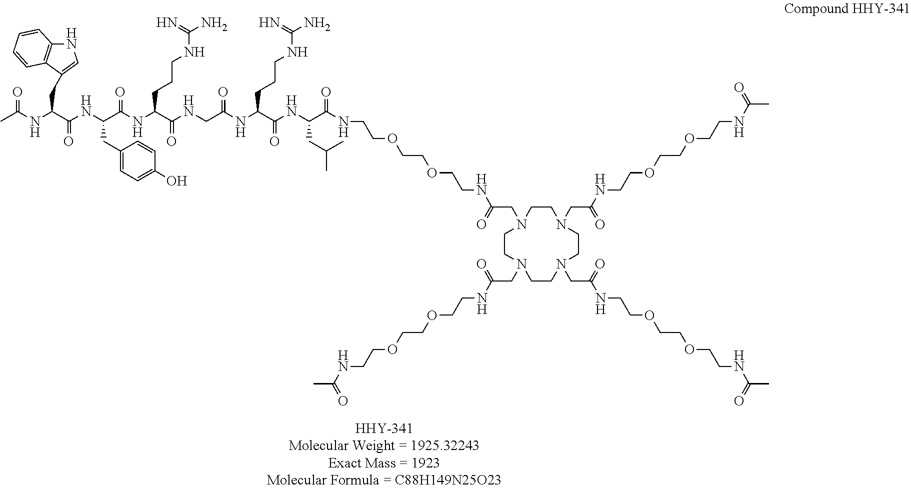 [SMILES: CC(=O)NCCOCCOCCNC(=O)CN1CCN(CC(=O)NCCOCCOCCNC(C)=O)CCN(CC(=O)NCCOCCOCCNC(=O)[C@H](CC(C)C)NC(=O)[C@H](CCCNC(=N)N)NC(=O)CNC(=O)[C@H](CCCNC(=N)N)NC(=O)[C@H](Cc2ccc(O)cc2)NC(=O)[C@H](Cc2c[nH]c3ccccc23)NC(C)=O)CCN(CC(=O)NCCOCCOCCNC(C)=O)CC1]

For removal of all of the protection groups, compound HHY-340 (52 mg, 0.02 mmol) was dissolved in 2 ml 95% TFA/H₂O and the reaction mixture was stirred for 3 hours under argon at room temperature (LC-MS determination). The solvent was 3 times co-evaporated with toluene, purified by HPLC to give HHY-341 (17 mg, yield: 44%) as a white powder. ESI-MS found: [M+H]²⁺=963.7.

HRMS calcd. for C₈₈H₁₄₉N₂₅O₂₃: 1924.12581. found: 1924.12671 ([M+3H]³⁺=642.38285).

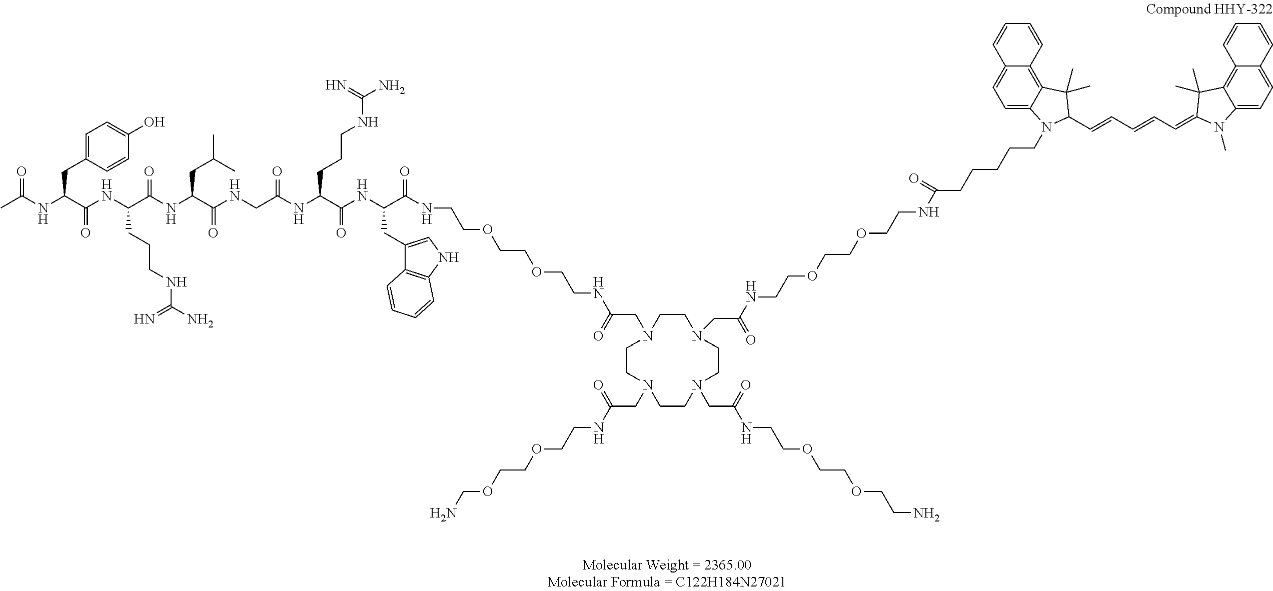 [SMILES: CC(=O)N[C@@H](Cc1ccc(O)cc1)C(=O)N[C@@H](CCCNC(=N)N)C(=O)N[C@@H](CC(C)C)C(=O)NCC(=O)N[C@@H](CCCNC(=N)N)C(=O)N[C@@H](Cc1c[nH]c2ccccc12)C(=O)NCCOCCOCCNC(=O)CN1CCN(CC(=O)NCCOCCOCN)CCN(CC(=O)NCCOCCOCCN)CCN(CC(=O)NCCOCCOCCNC(=O)CCCCCN2c3ccc4ccccc4c3C(C)(C)C2/C=C/C=C/C=C2/N(C)c3ccc4ccccc4c3C2(C)C)CC1]

The preparation of control probe HHY-322 was performed under similar conditions as those described for probe HHY-312 using scrambled peptide sequence YRLGRW. ESI-MS found: [M+2H]³⁺=789.0

FIRMS calcd. for C₁₂₂H₁₈₄N₂₇O₂₁: 2363.41543. found: 2363.41368 ([M+4H]⁵⁺=473.48856).

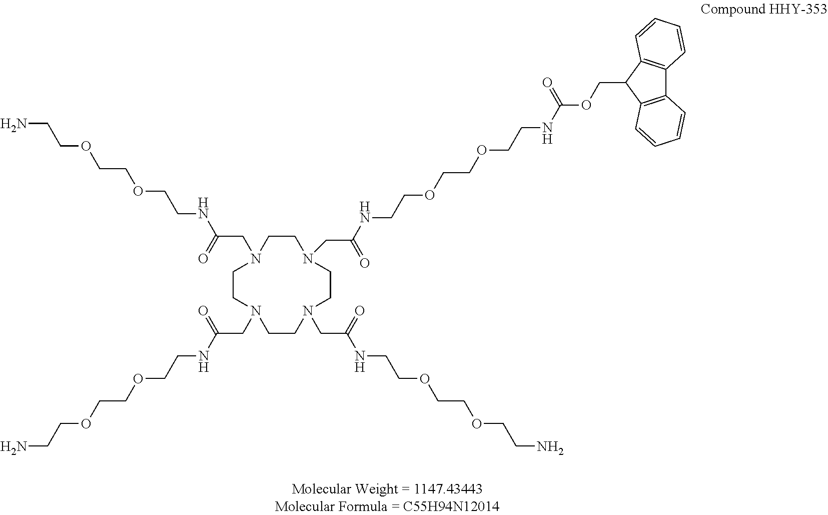 [SMILES: NCCOCCOCCNC(=O)CN1CCN(CC(=O)NCCOCCOCCN)CCN(CC(=O)NCCOCCOCCNC(=O)OCC2c3ccccc3-c3ccccc32)CCN(CC(=O)NCCOCCOCCN)CC1]

For removal of all of the protection Boc groups, compound HHY-335 was dissolved in 10 ml 50% TFA/CH₂Cl₂ and the reaction mixture was stirred for 1 hour under argon at room temperature. The reaction was monitored by LC-MS. After the reaction was complete, the solvent was 3 times co-evaporated with toluene. The crude product was used without further purification in the next step. ESI-MS calcd. for C₅₅H₉₄N₁₂O₁₄: 1147.43. found: [M+H]⁺=1148.9.

Synthesis of Scrambled Control of Example 2: Compound HHY-341Gd

AcYRLGRW-DOTA-[Gd3⁺]

ESI-MS calcd. for C₈₈H₁₄₉N₂₅O₂₃Gd: 2082.57. found: 2083.4 ([M+H]⁴⁺=521.1).

Synthesis of Key Intermediate for 3-Peptides Conjugates

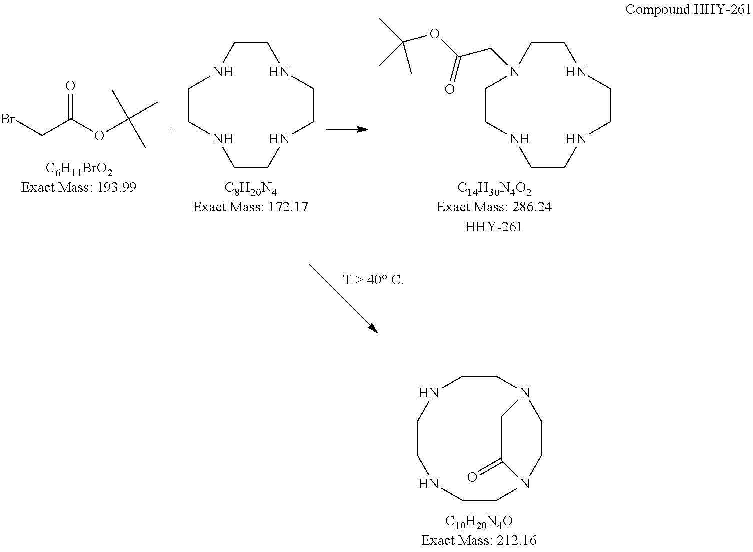

To a stirred solution of 1,4,7,10-tetraazacyclododecane (cyclen) (3.44 g, 20 mmol) in CHCl₃ (40 mL) was added t-butylbromoacetate (0.5 eq) in CHCl₃ (10 mL) within 1 h at 0° C. Stirring was continued for an additional hour. The solution became cloudy (cyclen.HBr). TLC control showed complete disappearance of the t-butylbromoacetate. The precipitate was filtered and the filtrate concentrated in vacuo. The resulting oil was purified by HPLC (95% H₂O-60% CH₃CN) to yield HHY-261 (1.43 g, 50%) as a colorless (Tetrahedron Letters. 2006, 47, 5985-5988). ESI-MS found: [M+H]⁺=287.3.

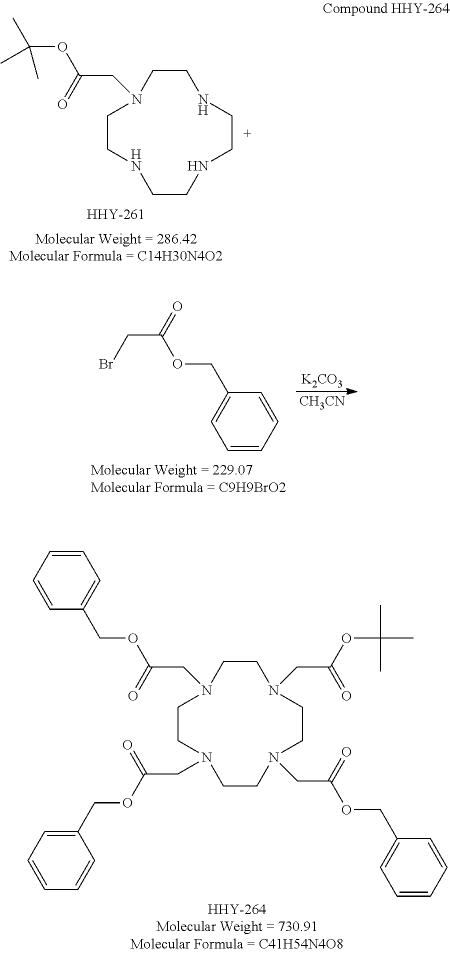

To a suspension of HHY-261 (1.43 g) and powdered K₂CO₃ (8 eq) in 100 mL acetonitrile, a solution of benzyl 2-bromoacetate (4 eq) in 50 mL acetonitrile was added dropwise over a period of one hour under argon and the reaction mixture was allowed to stand at room temperature for overnight. The reaction was monitored by LC-MS. After the reaction was complete, the K₂CO₃ was filtered away and the acetonitrile removed by evaporation. The reaction was diluted with DCM and washed three times with water, and three times with brine. The product was purified by silica gel chromatography with a gradient from 3% to 15% MeOH in DCM. Yield: 1.46 g, 40%. ESI-MS found: [M+H]⁺=731.5.

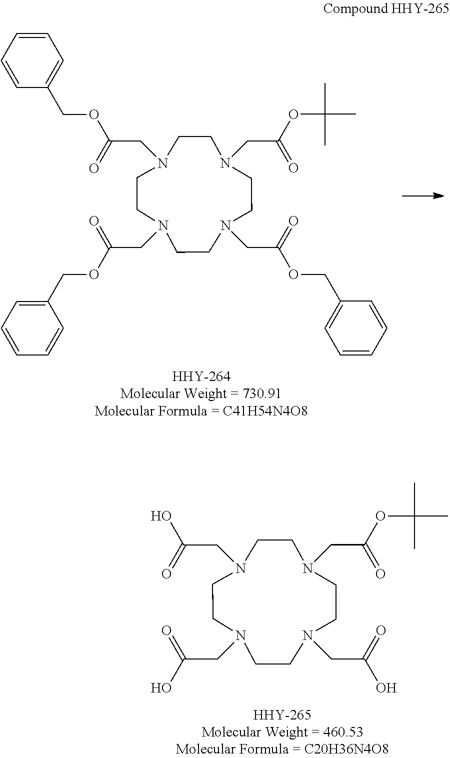 [SMILES: CC(C)(C)OC(=O)CN1CCN(CC(=O)OCc2ccccc2)CCN(CC(=O)OCc2ccccc2)CCN(CC(=O)OCc2ccccc2)CC1 ; CC(C)(C)OC(=O)CN1CCN(CC(=O)O)CCN(CC(=O)O)CCN(CC(=O)O)CC1]

HHY-264 (1.46 g, 2 mmol) was hydrogenolyzed over 10% Pd on carbon (50 mg) in MeOH (50 mL) for 12 h. The reaction was monitored by LC-MS. After the reaction was complete, the Pd/C was removed by filtration and the MeOH removed by evaporation. The crude product was used without further purification in the next step. ESI-MS found: [M+H]⁺−461.3.

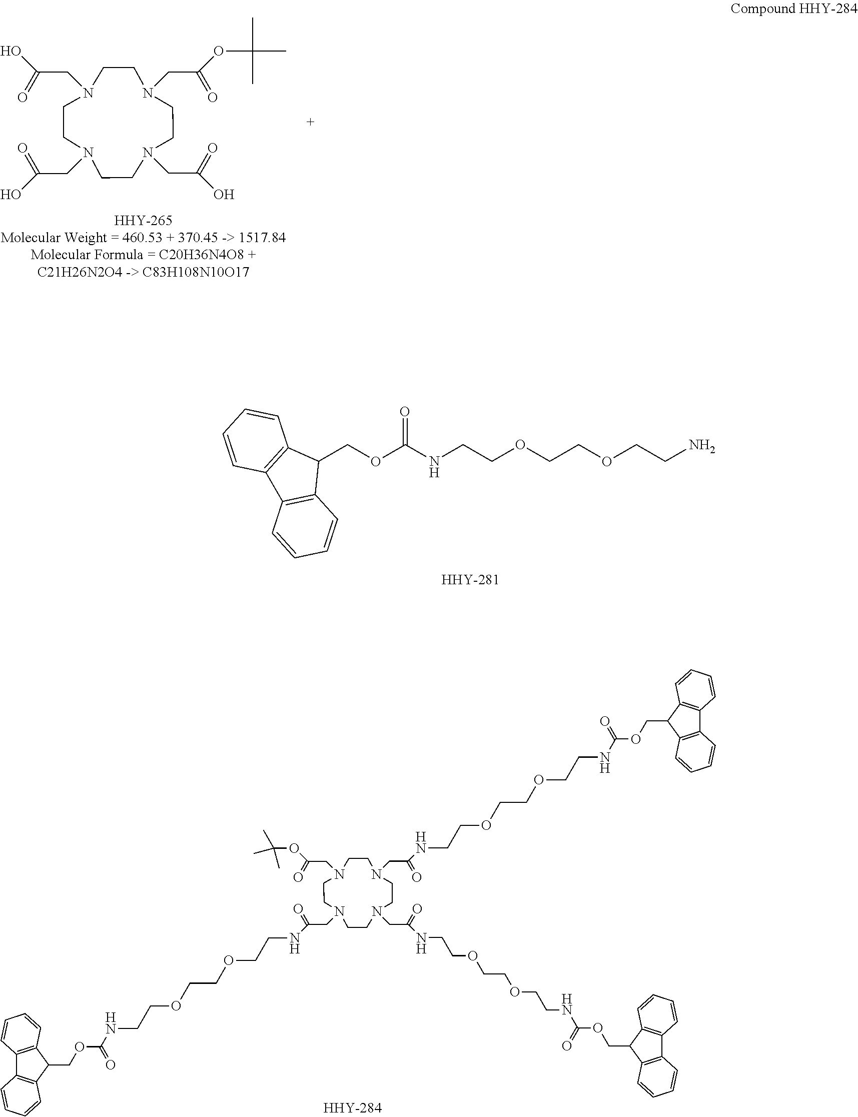 [SMILES: CC(C)(C)OC(=O)CN1CCN(CC(=O)O)CCN(CC(=O)O)CCN(CC(=O)O)CC1 ; NCCOCCOCCNC(=O)OCC1c2ccccc2-c2ccccc21 ; CC(C)(C)OC(=O)CN1CCN(CC(=O)NCCOCCOCCNC(=O)OCC2c3ccccc3-c3ccccc32)CCN(CC(=O)NCCOCCOCCNC(=O)OCC2c3ccccc3-c3ccccc32)CCN(CC(=O)NCCOCCOCCNC(=O)OCC2c3ccccc3-c3ccccc32)CC1]

1 equiv. HHY-265 (230 mg, 0.5 mmol), 3.1 equiv. HATU (589 mg, 1.55 mmol) and 12 equiv. DIPEA (1.05 ml, 6 mmol) were dissolved in 10 ml DMF. After 10 min, 4 equiv. HHY-281 (741 mg, 2 mmol) was added to the reaction mixture and stirred 30 min. under argon at room temperature. The solution was diluted with EtOAc, and washed sequentially with 1N HCl (3 times), saturated NaHCO₃, and brine. The organic layer was dried over MgSO₄. Removal of the volatiles in vacuo provided pale yellow oil, which was purified by HPLC with CH₃CN—H₂O to give HHY-284 (510 mg, 67%) as a white powder. ESI-MS found: [M+2H]²⁺=760.2.

¹H NMR (d₆-DMSO, 400 MHz): δ 8.54 (bs, 2H, —NH), 8.09 (bs, 2H, —NH), 7.88 (d, J=7.6 Hz, 6H, —Ar), 7.74 (bs, 2H, —NH), 7.67 (d, J=7.6 Hz, 6H, —Ar), 7.41 (t, J=7.5 Hz, 6H, —Ar), 7.31 (t, J=7.5 Hz, 6H, —Ar), 4.29 (d, J=6.8 Hz, 6H, —CH₂O—), 4.20 (t, J=6.8 Hz, 3H, —CHAr—), 3.50-2.99 (m, 60H, —CH₂—), 1.39 (s, 9H, —CH₃).

¹³C NMR (d₆-DMSO, 100 MHz): δ 156.2, 143.8, 127.6, 127.0, 125.1, 120.1, 69.4, 69.0, 68.7, 65.3, 46.7, 40.1, 35.7, 27.8.

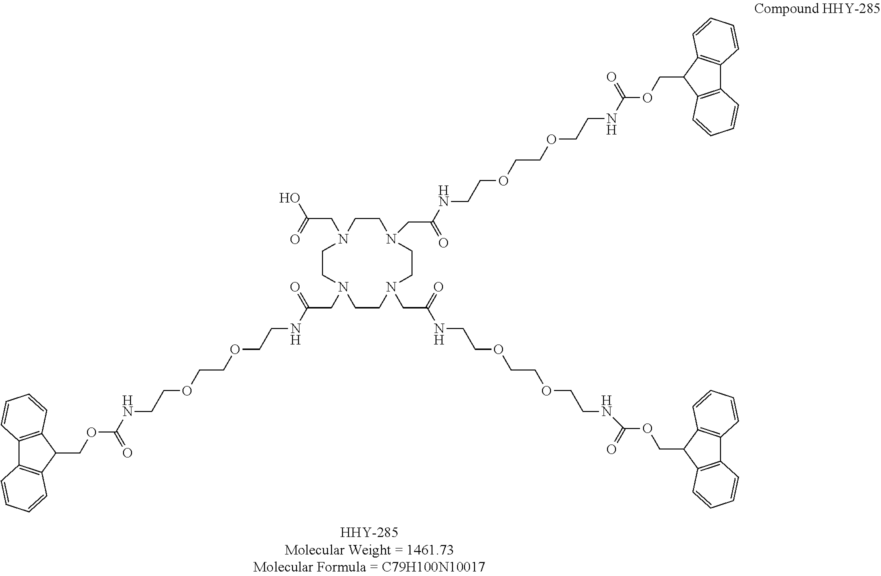 [SMILES: O=C(O)CN1CCN(CC(=O)NCCOCCOCCNC(=O)OCC2c3ccccc3-c3ccccc32)CCN(CC(=O)NCCOCCOCCNC(=O)OCC2c3ccccc3-c3ccccc32)CCN(CC(=O)NCCOCCOCCNC(=O)OCC2c3ccccc3-c3ccccc32)CC1]

For removal of the tBu-group, compound HHY-284 (152 mg, 0.1 mmol) was dissolved in 3 ml 95% TFA/H₂O and the reaction mixture was stirred for 2 hours under argon at room temperature. The reaction was monitored by LC-MS. After the reaction was complete, the solvent was 3 times co-evaporated with toluene. The crude product was used without further purification in the next step.

ESI-MS found: [M+2H]²⁺=731.6.

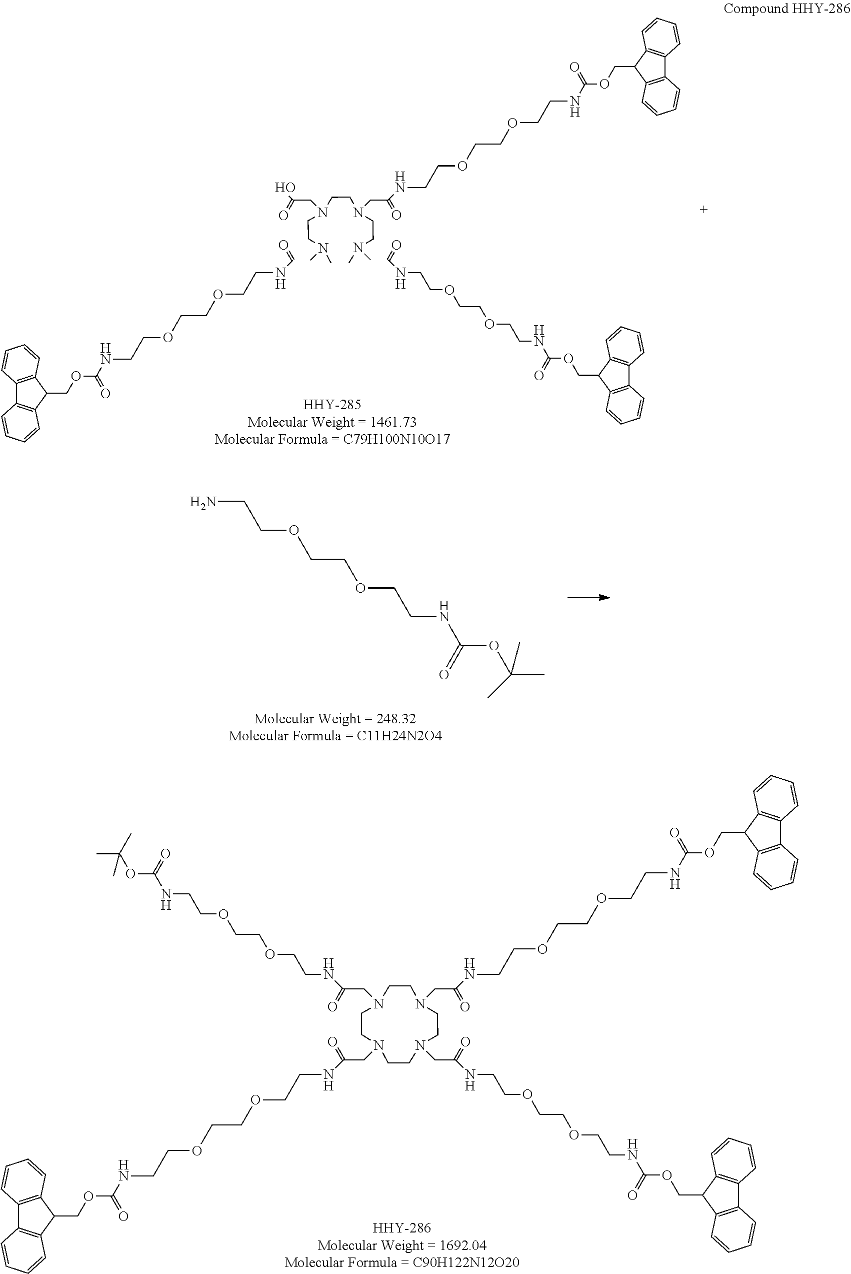

1 equiv. HHY-285 (146 mg, 0.1 mmol), 1.1 equiv. HAM (42 mg, 0.11 mmol) and 6 equiv. DIPEA (105 μl, 0.6 mmol) were dissolved in 5 ml DMF. After 30 min, 1.5 equiv. Boc-1-amino-3,6-dioxa-8-octanediamine (37 mg, 0.15 mmol) was added to the reaction mixture and stirred 30 min. under argon at room temperature. The reaction was monitored by LC-MS. After the reaction was complete, the reaction solution was directly and immediately purified by HPLC to give HHY-286 (113 mg, 67%) as a white powder. ESI-MS found: [M+2H]²⁺=846.7.

¹H NMR (d₆-DMSO, 400 MHz): δ 8.34 (bs, 4H, −8.20, —NH), 7.88 (d, J=7.6 Hz, 6H, —Ar), 7.68 (d, J=7.6 Hz, 6H, —Ar), 7.42 (t, J=6.8 Hz, 6H, —Ar), 7.34-7.30 (m, 9H, —Ar, —NH), 6.74 (t, J=4.78 Hz, 1H, —NH), 4.29 (d, J=6.2 Hz, 6H, —CH₂O—), 4.21 (t, J=6.2 Hz, 3H, —CHAr—), 3.62-3.04 (m, 72H, —CH₂—), 1.37 (bs, 9H, —CH₃).

¹³C NMR (d₆-DMSO, 100 MHz): δ 167.2, 158.3, 157.9, 157.6, 156.2, 155.6, 143.8, 140.7, 127.6, 127.0, 125.1, 120.7, 120.1, 117.8, 114.9, 111.9, 77.6, 69.5, 69.4, 69.39, 69.1, 68.8, 65.3, 54.9, 54.6, 49.6, 40.1, 38.7, 31.3, 28.2.

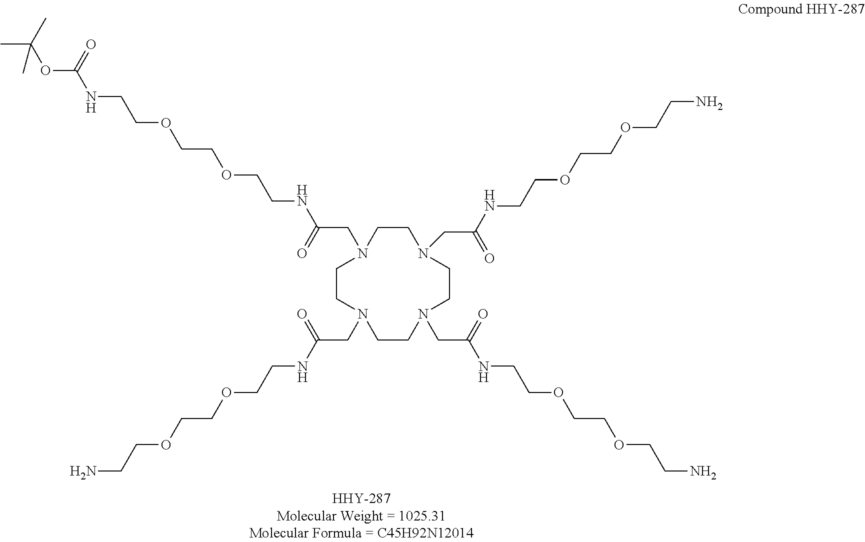 [SMILES: CC(C)(C)OC(=O)NCCOCCOCCNC(=O)CN1CCN(CC(=O)NCCOCCOCCN)CCN(CC(=O)NCCOCCOCCN)CCN(CC(=O)NCCOCCOCCN)CC1]

For removal of the Fmoc-group compound HHY-286 (51 mg, 0.03 mmol) was dissolved in 2 ml Et₂NH/DMF (1/4) and the reaction mixture was stirred for 10 min. at room temperature. The reaction was monitored by LC-MS. After the reaction was complete, the solvent was removed under reduced pressure. The crude product was used without further purification in the next step. ESI-MS found: [M+H]⁺=1026.8.

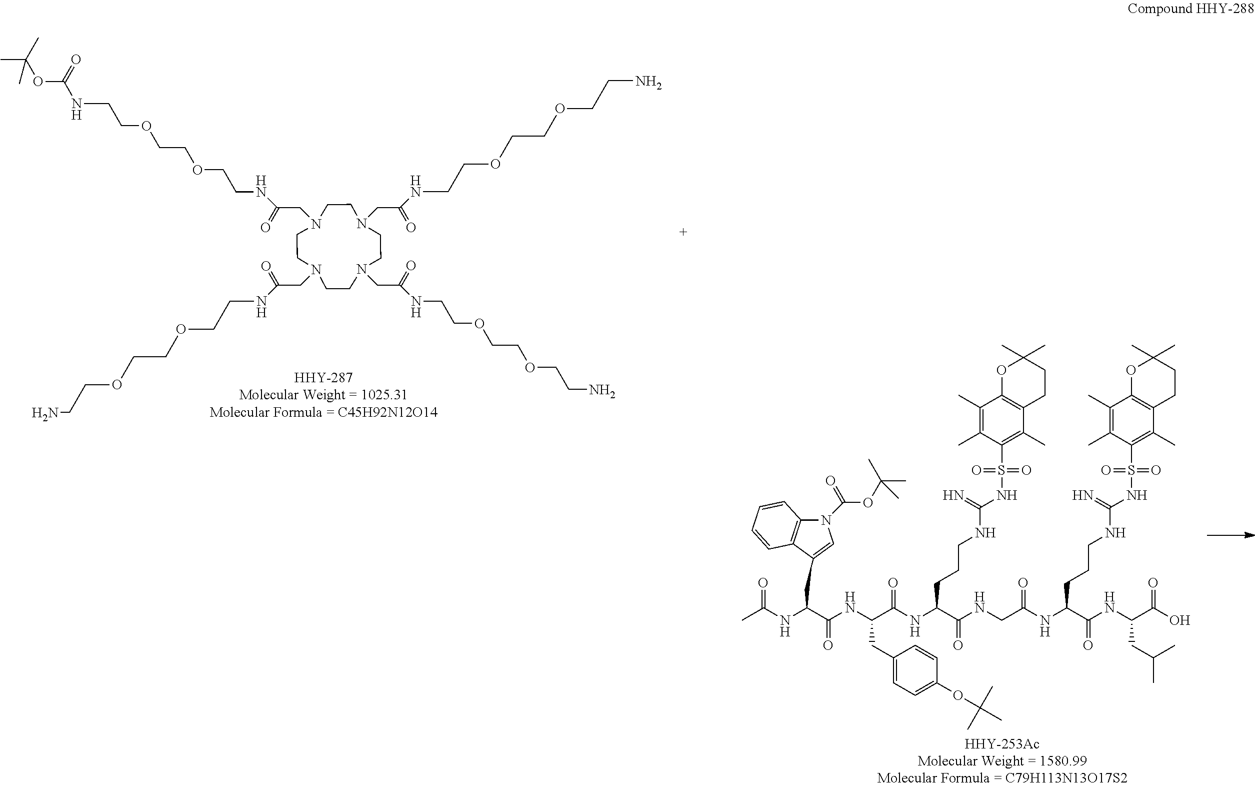 [SMILES: CC(C)(C)OC(=O)NCCOCCOCCNC(=O)CN1CCN(CC(=O)NCCOCCOCCN)CCN(CC(=O)NCCOCCOCCN)CCN(CC(=O)NCCOCCOCCN)CC1 ; CC(=O)N[C@@H](Cc1cn(C(=O)OC(C)(C)C)c2ccccc12)C(=O)N[C@@H](Cc1ccc(OC(C)(C)C)cc1)C(=O)N[C@@H](CCCNC(=N)NS(=O)(=O)c1c(C)c(C)c2c(c1C)CCC(C)(C)O2)C(=O)NCC(=O)N[C@@H](CCCNC(=N)NS(=O)(=O)c1c(C)c(C)c2c(c1C)CCC(C)(C)O2)C(=O)N[C@@H](CC(C)C)C(=O)O]

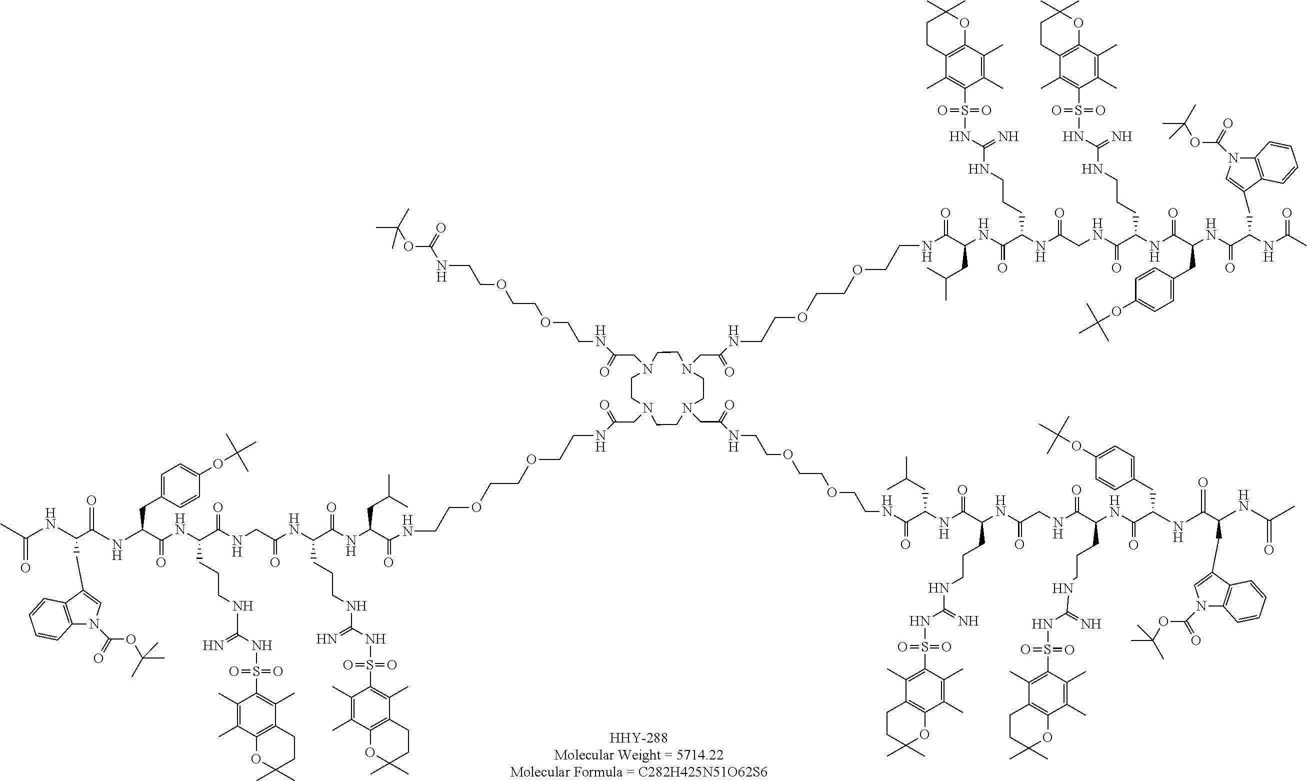

4 equiv. peptide WYRGRL (HHY-253Ac) (190 mg, 0.12 mmol), 4 equiv. HATU (46 mg, 0.12 mmol) and 12 equiv. DIPEA (63 μl, 0.36 mmol) were dissolved in 3 ml DMF. After 20 min, 1 equiv. HHY-287 (31 mg, 0.03 mmol) was added to the reaction mixture. After stirred 30 min under argon at room temperature, the solution was diluted with EtOAc, and washed sequentially with 1N HCl, saturated NaHCO₃, and brine. The organic layer was dried over MgSO4. Removal of the volatiles in vacuo provided pale yellow oil, which was purified by HPLC with CH₃CN—H₂O (20-95%) to give HHY-288 (105 mg, 61%) as a white powder. ESI-MS found: [M+4H]⁴⁺=0.1429.6.

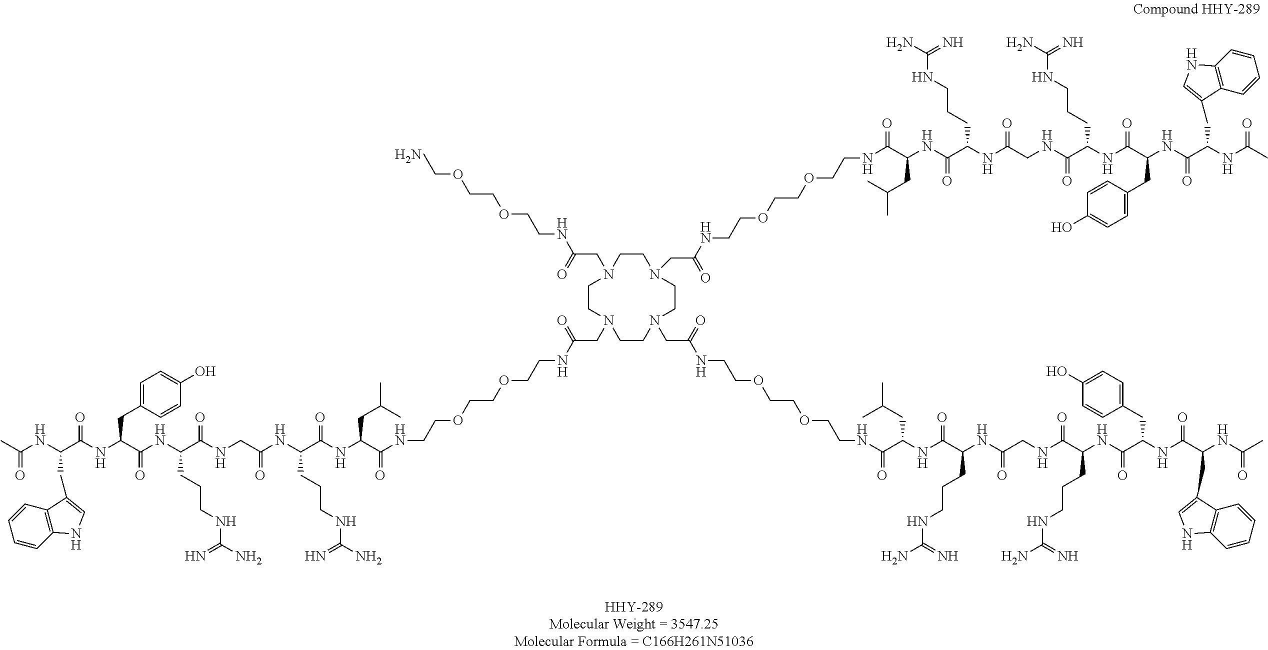 [SMILES: CC(=O)N[C@@H](Cc1c[nH]c2ccccc12)C(=O)N[C@@H](Cc1ccc(O)cc1)C(=O)N[C@@H](CCCNC(=N)N)C(=O)NCC(=O)N[C@@H](CCCNC(=N)N)C(=O)N[C@@H](CC(C)C)C(=O)NCCOCCOCCNC(=O)CN1CCN(CC(=O)NCCOCCOCCN)CCN(CC(=O)NCCOCCOCCNC(=O)[C@H](CC(C)C)NC(=O)[C@H](CCCNC(=N)N)NC(=O)CNC(=O)[C@H](CCCNC(=N)N)NC(=O)[C@H](Cc2ccc(O)cc2)NC(=O)[C@H](Cc2c[nH]c3ccccc23)NC(C)=O)CCN(CC(=O)NCCOCCOCCNC(=O)[C@H](CC(C)C)NC(=O)[C@H](CCCNC(=N)N)NC(=O)CNC(=O)[C@H](CCCNC(=N)N)NC(=O)[C@H](Cc2ccc(O)cc2)NC(=O)[C@H](Cc2c[nH]c3ccccc23)NC(C)=O)CC1]

For removal of all of the protection groups, compound HHY-288 (29 mg, 5 μmol) was dissolved in 3 ml 95% TFA/H₂O and the reaction mixture was stirred for 3 hours under argon at room temperature. The reaction was monitored by LC-MS. After the reaction was complete, the solvent was 3 times co-evaporated with toluene. The crude product was used without further purification in the next step. ESI-MS found: [M+4H]⁴⁺=887.9.

### EXAMPLE 6

### Compound HHY-290

Targeting Peptide Fluorescent Conjugate (AcWYRGRL)₃-DOTA-Cy5.5

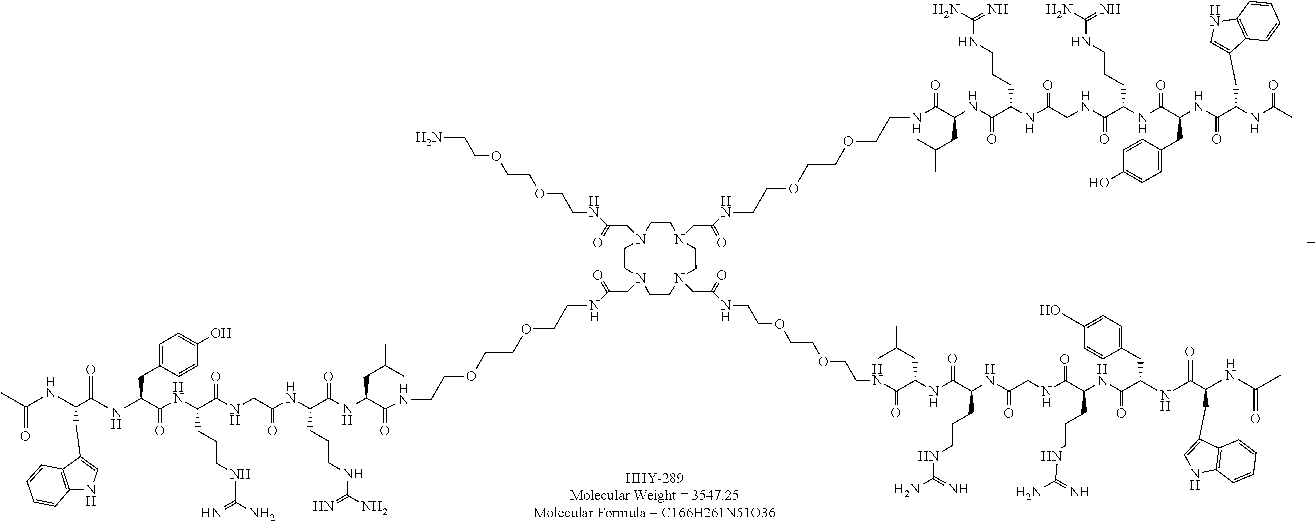 [SMILES: CC(=O)N[C@@H](Cc1c[nH]c2ccccc12)C(=O)N[C@@H](Cc1ccc(O)cc1)C(=O)N[C@@H](CCCNC(=N)N)C(=O)NCC(=O)N[C@@H](CCCNC(=N)N)C(=O)N[C@@H](CC(C)C)C(=O)NCCOCCOCCNC(=O)CN1CCN(CC(=O)NCCOCCOCCN)CCN(CC(=O)NCCOCCOCCNC(=O)[C@H](CC(C)C)NC(=O)[C@H](CCCNC(=N)N)NC(=O)CNC(=O)[C@H](CCCNC(=N)N)NC(=O)[C@H](Cc2ccc(O)cc2)NC(=O)[C@H](Cc2c[nH]c3ccccc23)NC(C)=O)CCN(CC(=O)NCCOCCOCCNC(=O)[C@H](CC(C)C)NC(=O)[C@H](CCCNC(=N)N)NC(=O)CNC(=O)[C@H](CCCNC(=N)N)NC(=O)[C@H](Cc2ccc(O)cc2)NC(=O)[C@H](Cc2c[nH]c3ccccc23)NC(C)=O)CC1]

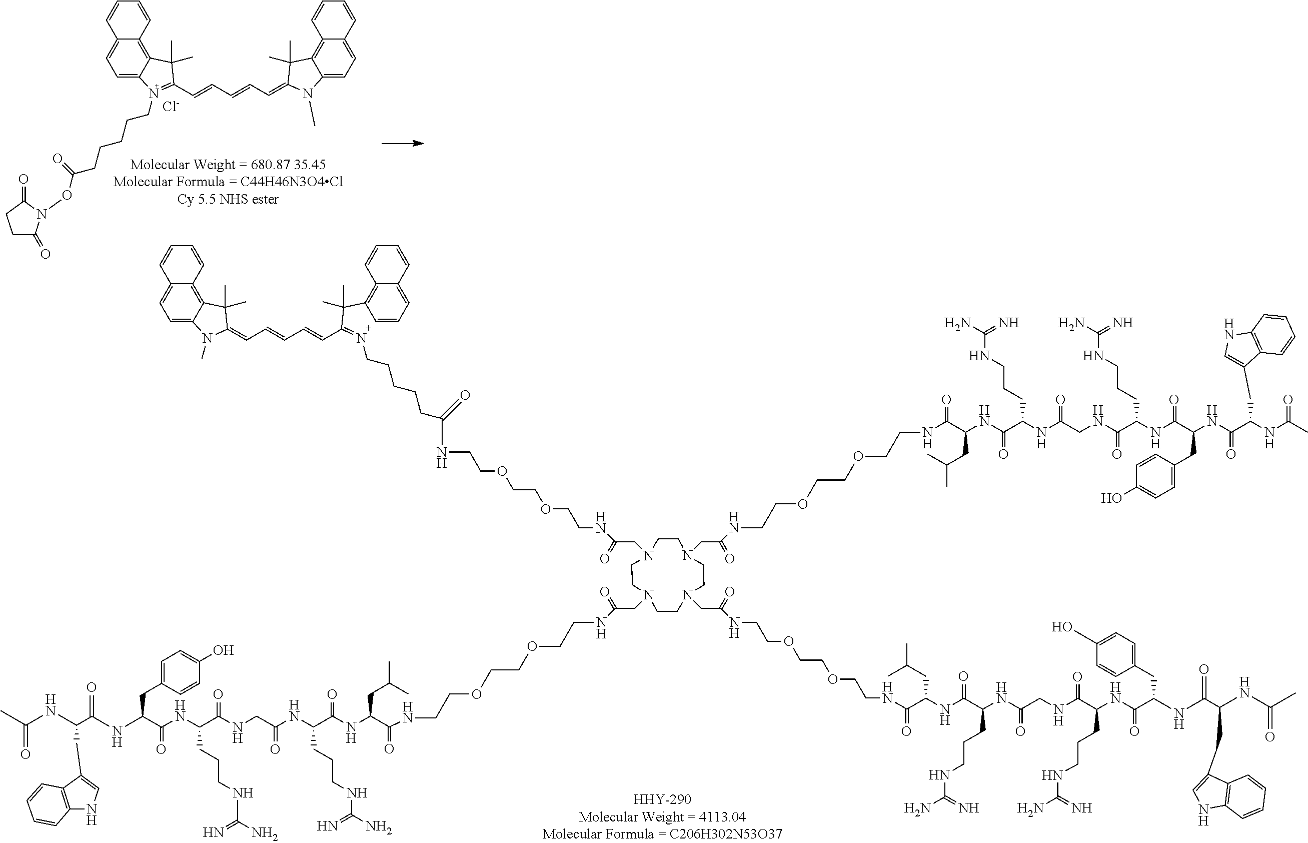

HHY-289 (15 mg, 4.2 μmol) was dissolved in 1.5 ml DMF, followed by the addition of 1.3 equiv. Cy5.5 NHS ester (4 mg, 5.5 μmol) and 6 equiv. DIPEA (5 μl, 25 μmol). The reaction mixture was stirred under argon at room temperature for 12 h. After the reaction was complete, the solvent was removed under reduced pressure. After the reaction was complete, the reaction solution was directly purified by HPLC to give to give HHY-290 (13 mg, Yield 75%) as a blue powder.

ESI-MS found: [M+3H]⁴⁺=1029.0. HRMS calcd. for C₂₀₆H₃₀₂N₅₃O₃₇: 4110.33738. found: 4110.33888 ([M+5H]⁶⁺=685.89588).

### EXAMPLE 7

### Compound HHY-299

Targeting Peptide Drug Conjugate (AcWYRGRL)₃-DOTA-Pepstatin A

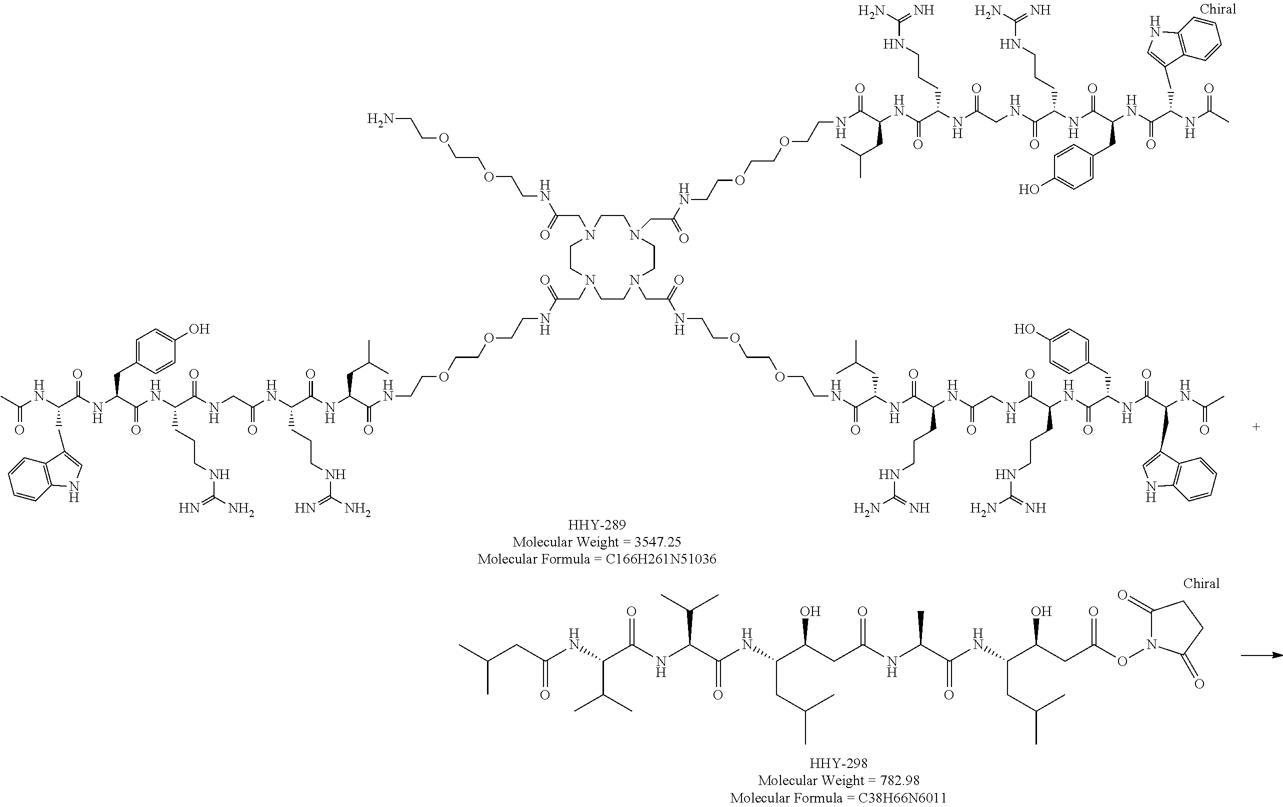

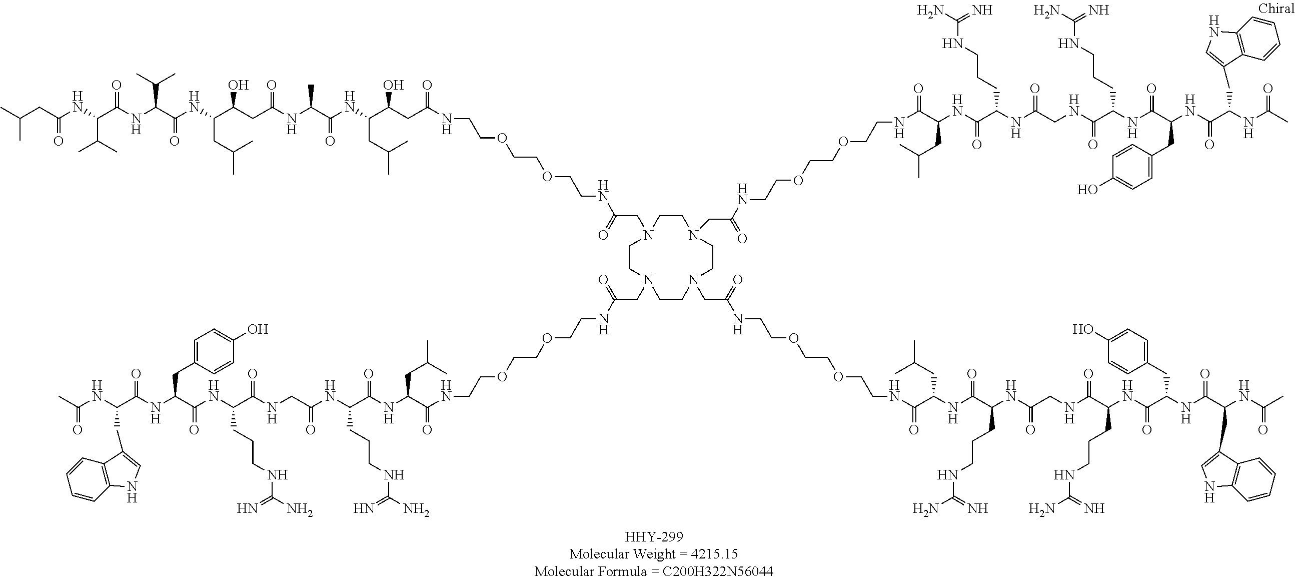

HHY-289 (10 mg, 2.8 μmol) was dissolved in 1.5 ml DMF, followed by the addition of 1.5 equiv. pepstatin A NHS ester HHY-298 (3.3 mg, 4.2 μmol) and 6 equiv. DIPEA (3 μl, 17 μmol). The reaction mixture was stirred under argon at room temperature for 12 h. The solvent was directly purified by preparative HPLC to give HHY-299 (3.2 mg, Yield 27.2%) as a white powder.

ESI-MS calcd. for C₂₀₀H₃₂₂N₅₆O₄₄: 4215.2. found: [M+4H]⁴⁺=1054.7.

Synthesis of Scrambled Control of Example 6: Compound HHY-293 (AcYRLGRW)₃-DOTA-Cy5.5

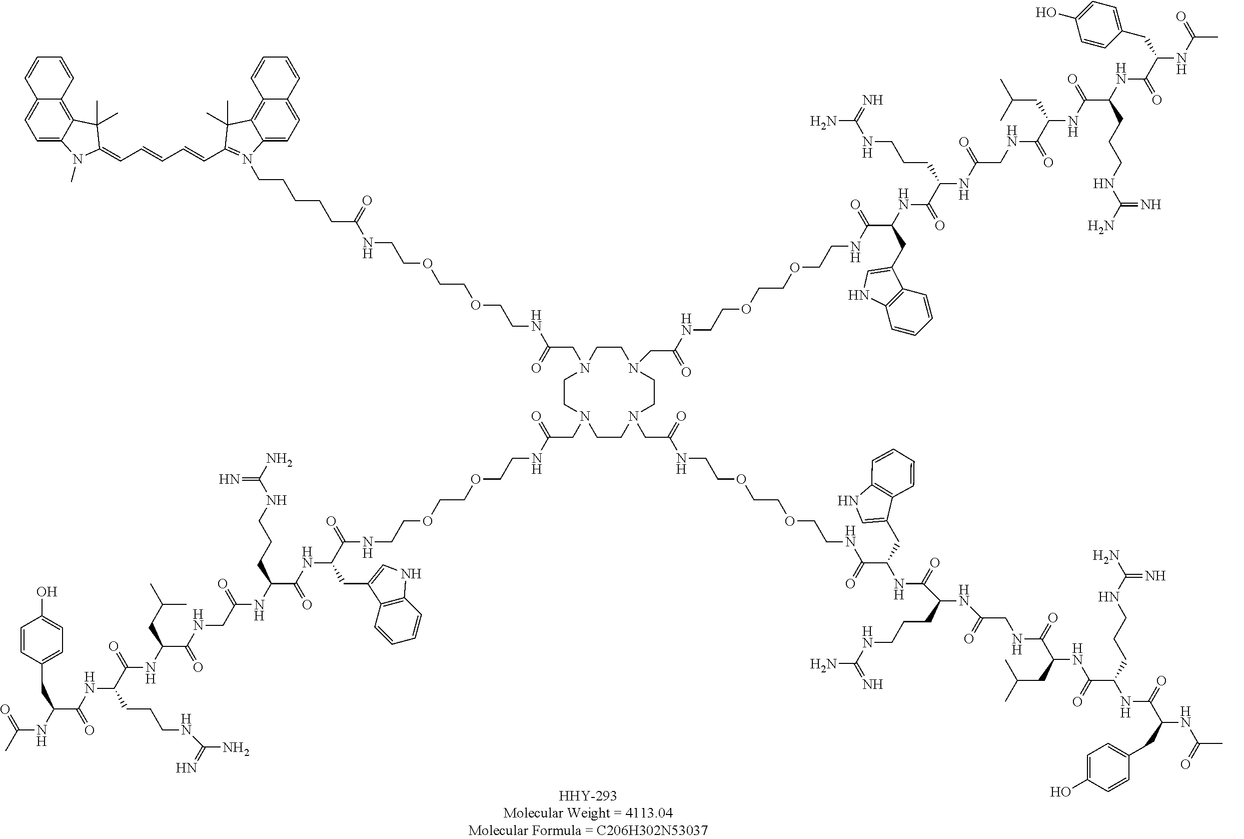

The preparation of control probe HHY-293 was performed under similar conditions as those described for probe HHY-290 using scrambled peptide sequence YRLGRW. ESI-MS found: [M+3H]⁴⁺=1029.1.

HRMS calcd. for C₂₀₆H₃₀₂N₅₃O₃₇: 4110.33738. found: 4110.33798 ([M+5H]⁶⁺=685.89573).

Synthesis of Key Intermediate for 2-Peptides Conjugates

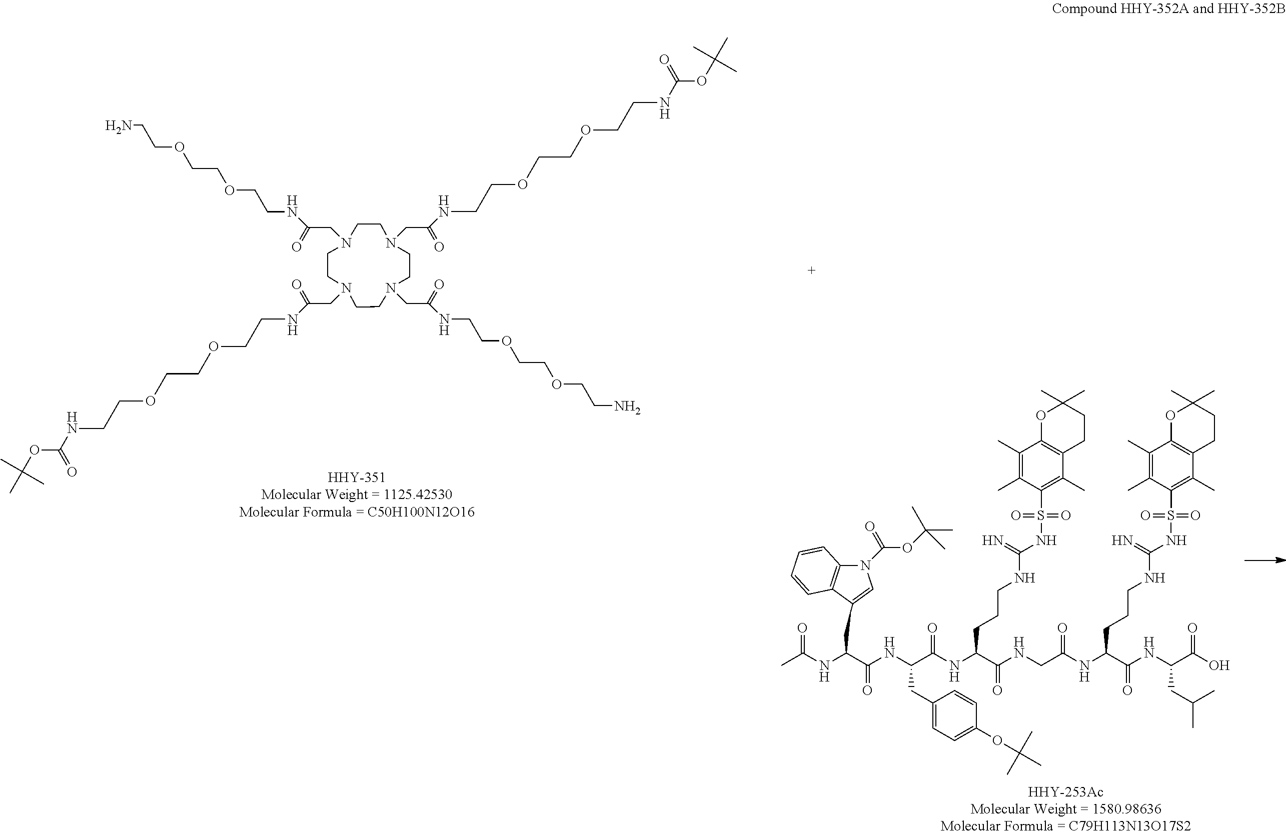

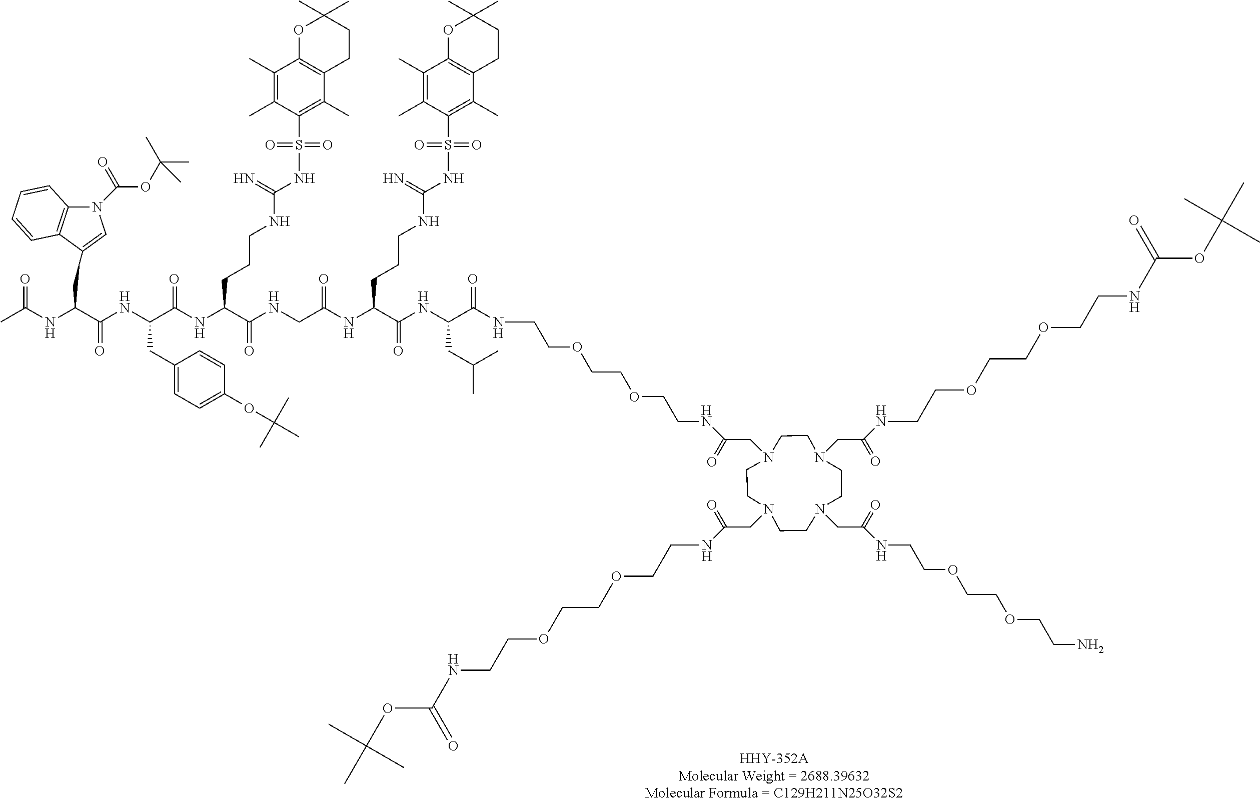 [SMILES: CC(=O)N[C@@H](Cc1cn(C(=O)OC(C)(C)C)c2ccccc12)C(=O)N[C@@H](Cc1ccc(OC(C)(C)C)cc1)C(=O)N[C@@H](CCCNC(=N)NS(=O)(=O)c1c(C)c(C)c2c(c1C)CCC(C)(C)O2)C(=O)NCC(=O)N[C@@H](CCCNC(=N)NS(=O)(=O)c1c(C)c(C)c2c(c1C)CCC(C)(C)O2)C(=O)N[C@@H](CC(C)C)C(=O)NCCOCCOCCNC(=O)CN1CCN(CC(=O)NCCOCCOCCNC(=O)OC(C)(C)C)CCN(CC(=O)NCCOCCOCCN)CCN(CC(=O)NCCOCCOCCNC(=O)OC(C)(C)C)CC1]

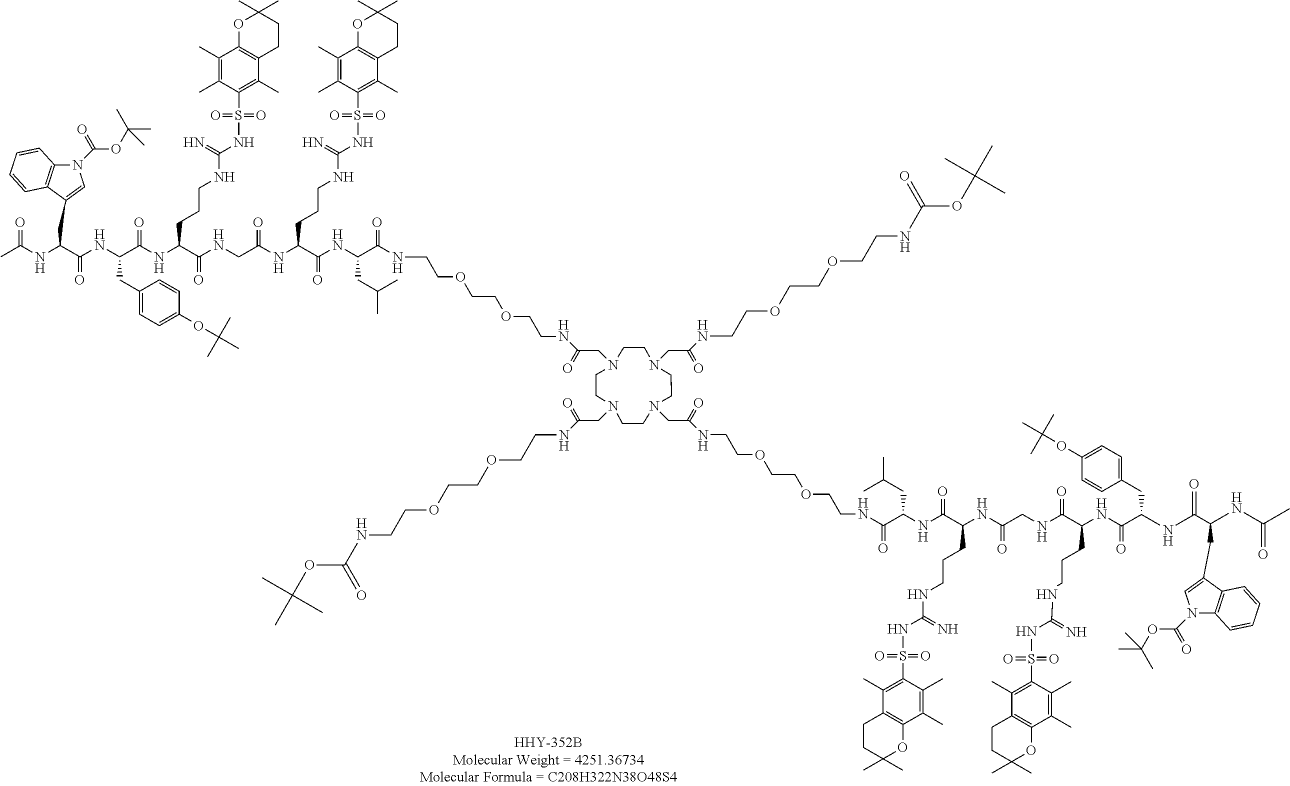

1 equiv. peptide WYRGRL (HHY-253Ac) (140 mg, 0.09 mmol), 1 equiv. HATU (34 mg, 0.09 mmol) and 3 equiv. DIPEA (46 μl, 0.37 mmol) were dissolved in 7 ml DMF. After 20 min, 1 equiv. HHY-351 (100 mg, 0.09 mmol) was added to the reaction mixture. After stirring for 2 h under argon at room temperature, the solution was diluted with EtOAc, and washed sequentially with saturated NaHCO₃ and brine. The organic layer was dried over MgSO₄. Removal of the volatiles in vacuo provided a pale yellow oil, which was purified by HPLC with CH₃CN—H₂O (20-95%) to give HHY-352A (129 mg, 54%) as a white powder, along with 75 mg (20%) of HHY-352B.

ESI-MS found: HHY-352A [M+2H]²⁺=1345.0. HHY-352B [M+3H]³⁺=1418.1.

To increase the yield of HHY-352B, 3 equivalents of HHY-253Ac, HATU and 9 eq. of DIPEA can be employed.

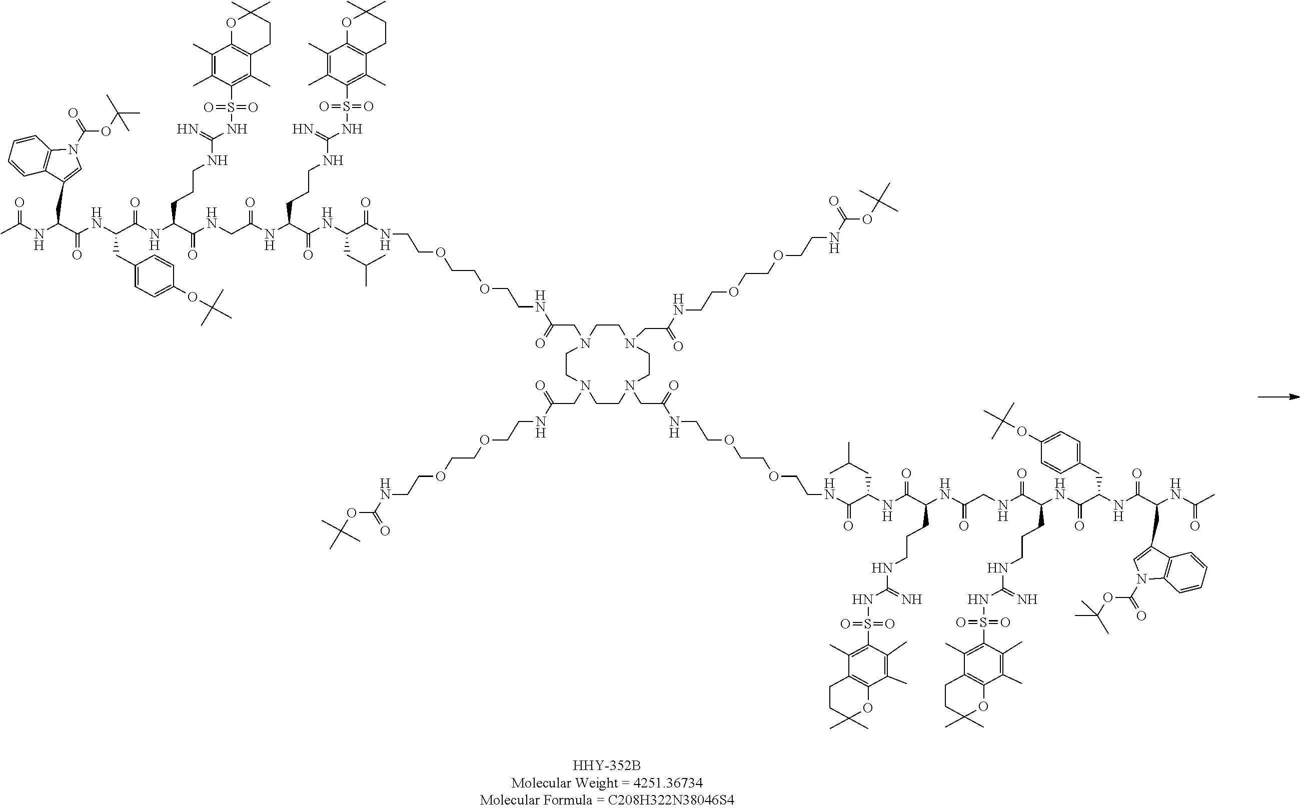

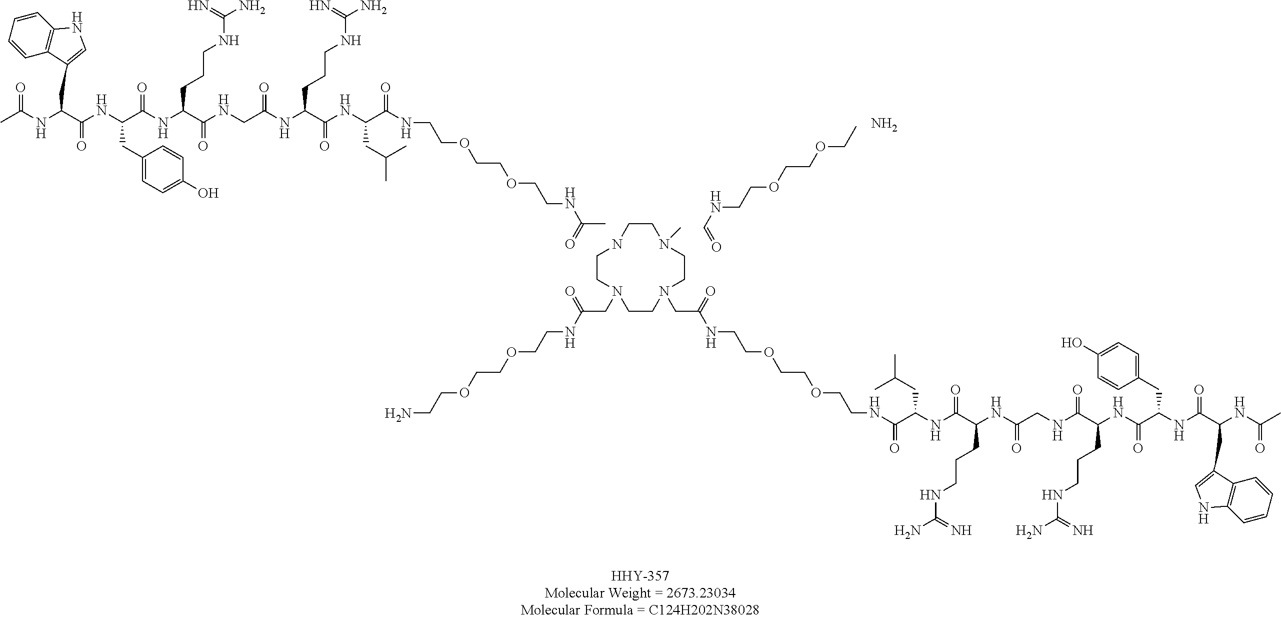

For removal of all of the protection groups, 75 mg HHY-352B was dissolved in 10 ml 95% TFA/H₂O and the reaction mixture was stirred for 3 hours under argon at room temperature. The reaction was monitored by LC-MS. After the reaction was complete, the solvent was 3 times co-evaporated with toluene. The crude product was purified by HPLC to give HHY-357 (yield: 28 mg, 60%) as a white powder.

ESI-MS found: [M+3H]³⁺=892.1

Pharmacological Testing

Measurement of In Vivo Half Life of Compounds in Mice

Male C57 BL/6J mice aged 10 weeks were purchased from Harlan Laboratories (Blackthorn, Bicester, UK). Mice were housed in groups of 6 in individually vented cages, maintained at 21±2° C. on a 12-hour light/dark cycle and with food provided ad libitum. All experimental protocols were performed in compliance with the UK Animals (Scientific Procedures) Act 1986 regulations for the handling and use of laboratory animals (Home Office project license PPL no: 70/7288).

One hundred nanomoles of each of the compounds were injected into the intra-articular space following induction of anaesthesia by gaseous anaesthetic (2% isofluorane and O₂). Planar fluorescence images of the knee joints was captured at the time points indicated by capturing the emission at 700 nm for 1 min after exposure at 630 nm under gaseous anaesthesia (2.5% isofluorane and O₂) in a Kodak In Vivo FX Pro (Carestream, Woodbridge, USA). Planar X-ray images were obtained by exposure to X-rays for 20 seconds for co-registration purposes.

To calculate the half lives of the compounds in the joint, fluorescence images were analysed using the Carestream MI software (version 5.1). Circular regions of interest (ROI) of 25 pixels in diameter were defined to encompass the knee joint, which was defined by co-registration of X-ray images. The mean fluorescence intensity (MFI) of the ROI was obtained. Fluorescence images shown are false coloured using the rainbow spectrum, with purple being the lowest value and red being the highest value, and the range indicated in the figures. The data was fitted to a two phase decay model to obtain two half lives, a fast half life (t1) representing loss of the compounds from the joint space, and a slower half life (t2), representing loss of compounds which has bound to the articular cartilage. Results are shown in Table 2. All statistical analysis was carried out using the Prism Software (Graphpad, La Jolla, USA).

TABLE 2

| | | First Half | Terminal |
|---|---|---|---|
| Probe | Structure | Life (h) | Half Life (h) |
| HHY-306 | AcNH-DOTA-Cy 5.5 | 0.5 | 9.8 |
| HHY-259B | AcWYRGRL-Cy 5.5 | 2.3 | 205.4 |
| HHY-258B | Scrambled (AcYRLGRW-Cy5.5) | 2.0 | 35.04 |
| HHY-312 | AcWYRGRL-DOTA-Cy 5.5 | 4.0 | 210.7 |
| HHY-322 | Scrambled AcYRLGRW-DOTA-Cy 5.5 | 1.67 | 12.5 |
| HHY-290 | (AcWYRGR)3-DOTA-Cy 5.5 | 8.9 | 1284 |
| HHY-293 | Scrambled (AcYRLGRW)3-DOTA-Cy 5.5 | 3.0 | 18.75 |

As an essentially non-binding control, probe HHY-306 with three capped amine groups was synthesized as reference.

Cryosectioning and Confocal Imaging

Knee joints were dissected from the mice and embedded in OCT (Sakura Finetek UK, Thatcham, UK) before freezing in isopentane (VWR, Leistershire, UK) cooled by liquid nitrogen, before storage overnight at −80° C. The joints were sectioned at a thickness of 10 μm using a Leica CM1900UV cryotome (Leica Biosystems, Milton Keynes, UK).

The cryo-sections were fixed in ice cold acetone (VWR, Leistershire, UK) before being air dried and blocked in 5% bovine serum albumin and 1% goat serum in PBS. The primary anti-heparan sulphate proteoglycan (perlecan) antibody (Millipore, Watford, UK) was incubated at a 1:100 in blocking solution overnight with the sections at 4° C. Alexa-488-conjugated goat anti-mouse antibodies (Life Technologies, Paisley, UK) was used as the secondary antibody. The cryosections were mounted using Prolong Gold (Life Technologies, Paisley, UK). Cryosections were viewed under the Nikon TE2000-U Ultraview confocal microscopes (Perkin Elmer, Seer Green, UK) using the 488 nm excitation laser with 525 nm emission filter to visualise the Alexa-488 label and the 640 nm excitation laser with 700 nm emission filter to visualise the Cy 5.5 label.

Ex Vivo Binding Assay with Pig Articular Cartilage Explants

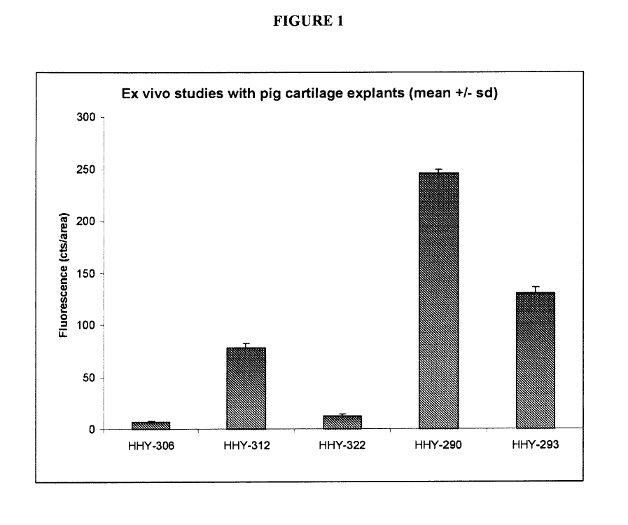

To evaluate the cartilage targeting affinities of these five DOTAM-derived compounds, we performed an ex vivo binding assay of these Cy 5.5-labeled probes with pig articular cartilage explants. In these ex vivo experiments, whole-depth pig articular cartilage blocks were incubated with 5 μM of each probe for 24 h, washed 3 times for 10 min each with PBS buffer at 37° C. to remove non-binding probes, then imaged using fluorescent microscopy. The intensity of the near infrared fluorescent signal from Cy 5.5 presents the relevant amount of these probes remaining in the cartilage. Probes that entered the cartilage were observed both in the matrix compartment and within chondrocytes. The pericellular matrix and the cell nuclei were substantially free of probes. It can be clearly seen that increasing the number of positive charges and collagen II targeting peptides, the probes were immobilized in the cartilage to a much greater extent. Quantification of probe accumulation by integrated fluorescence in the cartilage revealed dramatically more targeted probes than non-targeted (FIG. 1). There was an 11.2 fold mean increase of probe HHY-312 with one targeted peptide comparing with non-targeted HHY-306 and 6.0 fold comparing with non binding scrambled HHY-322 containing the same net charge as HHY-312 but lacking its specific collagen targeting properties. For the probe HHY-290 with three targeted peptides, there was a mean 36.6-fold greater accumulation in the cartilage than non-targeted HHY-306, 3.1-fold than HHY-312 (with two terminal charges and one targeting peptide) and 1.8-fold than HHY-293 with three scrambled peptides.

In Vivo Binding to Cartilage Using Optical Imaging

We further evaluated the binding capacity of the probes to cartilage in vivo using optical imaging. 0.1 μmol of each of the compounds were injected intra-articularly into the knees of mice, and in vivo fluorescent microscopy and X-ray were used to quantify localization and retention. The decay in signal was fitted to a two-phase exponential decay model to describe the rapid clearance of unbound probe from the joint and the slow clearance of bound probe from the cartilage (n=5).

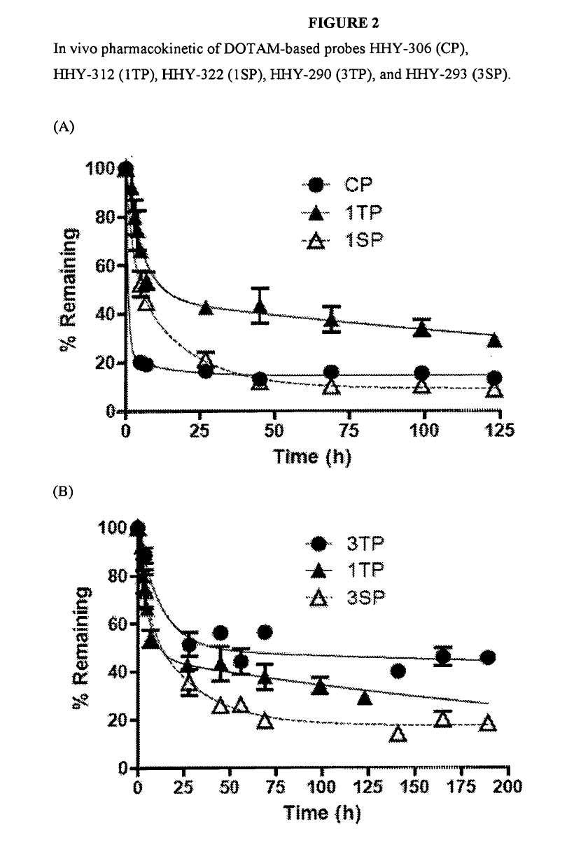

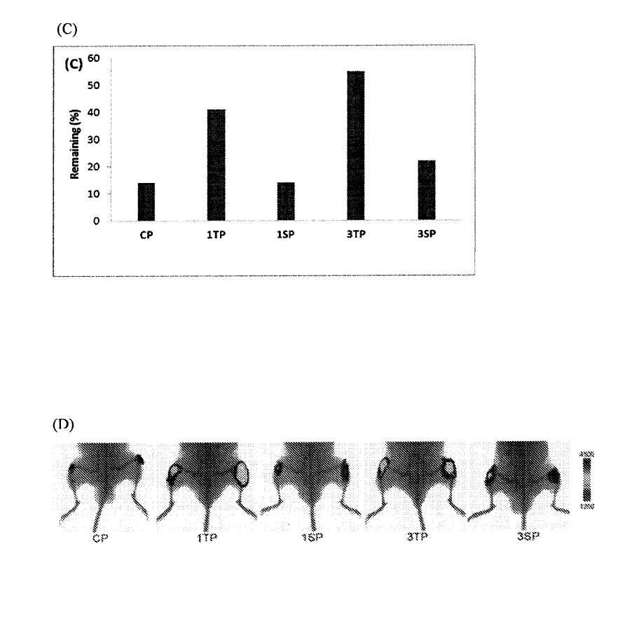

As shown in FIGS. 2(A) and 2(B), the probes showed a biphasic clearance pattern with a rapid initial clearance of unbound probes from the intra-articular space, followed by a slower second phase of cartilage-bound probe. The probe with the highest retention in cartilage, HHY-290 (3TP), retained 40% of the initial signal 8 days after injection, whereas the non-targeted HHY-306 (CP) was rapidly cleared, with less than 20% retention after 6 hours. The fitted second phase half-lives (t_{1/2beta}) of the targeting probes of 210 h for HHY-312 (1TP) and 1284 h for HHY-290 (3TP) were much longer compared to the scrambled control probes which had t_{1/2} of 13 h for HHY-322 (1SP) and 19 h for HHY-293 (3SP). FIG. 2(C) shows the remaining fluorescence signal from the knee joints of each probe after 48 hours, which also illustrates that the targeting probes HHY-312 (1TP) and HHY-290 (3TP) have a much longer retention time within the collagenous compartment of the knee joints than the control probes HHY-306 (CP), HHY-322 (1SP) and HHY-293 (3SP) due to their active targeting properties. FIG. 2(D) contains representative merged fluorescent and x-ray images showing the remaining amounts of probe remaining in the knee joints 48 h after intra-articular injection.

Encouragingly, the probes were well tolerated, with no adverse effects observed during the seven days of treatment.

Cathepsin D Enzyme Assay:

For quantification of Cathepsin D inhibitory activity, an enzymatic assay was used that measures the turnover of a quenched, fluorescenctly-labeled, Cathepsin D-cleavable, peptide substrate over time.

Specifically, 13 ng of purified Cathepsin D enzyme (Biomol; Cat.No. Se-199) was administered per well of a 96 well plate, substance/inhibitor of interest was added and the mixture incubated for 10 min. at 37° C. Then, the enzymatic reaction was started by addition of fluorescenctly-labeled, Cathepsin D-Substrat (10 mM (7-Methoxycoumarin-4-yl)-acetyl-Pro-Leu-Gly-Leu-(3 [2,4-dinitrophenyl]-L-2,3-diaminopropionyl)-Ala-Arg-NH2 (Bachem M-1895). The reaction volume amounts to 30 μl and contains 50 mM acetic acid, 200 mM NaCl and 0.003% Brij 35 and was adjusted to pH 4.5 with acetic acid. By cleavage of the peptide chain, the quencher 7-Methoxycoumarin is released and the emission of fluorescence dye is measured using excitation/emission wavelengths (330 nM/390 nM) on a Tecan Spectrafluor Plus—instrument at 37° C. for 10 min. For the determination of the compound dose response, a 10 mM DMSO stock solution was diluted and tested in a ten-point, three-fold dilution series run in duplicate beginning at 30 μM final concentration. Data were analyzed using a four-parameter curve fit with a fixed minimum and maximum experimentally defined as the average positive and negative controls on each plate. IC₅₀ values (in nM (micromol/liter)) for inhibition of Cathepsin D inhibitory determined in this assay are given in Table 3 below.

TABLE 3

| Tested compound | IC₅₀ [nM] |
|---|---|
| Control (compound HHY-325) | 2.9 |
| Pepstatin A | 0.55 |
| Example 7 (compound RHY-299) | 3.3 |
| Example 4 (compound HHY-327) | 2.1 |

After conjugation the compounds displays uncompromised activity on the target cathepsin D, although steric demand is tremendous.

In addition, the solubility of the compound HHY-327 (>5.92 mg/mL in phosphate buffer at pH 7.4) is increased compared to solubility of pepstatin A (0.82 mg/mL).

The loss of proteoglycans, mainly aggrecan, from articular cartilage is one of the initial hallmarks of the osteoarthritic pathogenesis. When cartilage is maintained at an acidic pH it has been shown that there is a rapid loss of aggrecan from the tissue and it has been suggested that the pH dependent activation of cathepsin D is responsible for this loss. By inhibition of cathepsin D with pepstatin A, the proteoglycan and cartilage degradation at pH 5 is attenuated.

In order to explore the cartilage targeting affinity of the conjugates in comparison to free pepstatin A as well as to determine the efficiency of these inhibitors on the aggrecan degradation in articular cartilage occurring during osteoarthritis, an ex vivo GAG release model with pig articular cartilage explant was carried out.

Preparation and Treatment of Cartilage Explants

The cartilage degradation model was performed using pig articular cartilage explants. Whole-depth pig articular cartilage was dissected into pieces (d=3 mm). Briefly, the knee joints were dissected for articular cartilage from 20-24 week-old pigs. These were then rested in Dulbecco's modified Eagle's medium (DMEM) containing 200 units/ml of penicillin and 10% (v/v) fetal bovine serum and 5% CO2 at 37° C. for 24 hours m before being washed twice with DMEM without serum. The concentration of pepstatin A/HHY-327 stock solution was 10 mM in DMSO. Inhibitors were diluted with DMEM buffer to 1 μM. Each cartilage piece was placed in one well of a round-bottomed 48-well plate with 1000 μl serum-free DMEM at pH 7.4, together with cathepsin D inhibitor pepstatin A or HHY-327. After incubation at 37° C. for 24 h, the samples were washed 3 times for 10 min each with DMEM buffer at 37° C., then incubated with 1 mL of DMEM at pH 7.4 as control or 0.1 M Tris-acetate buffer at pH 5.0. After 4 h, 8 h, 24 h, the conditioned media were harvested and stored at −20° C. until use. It should be noted that all experiments were performed in n=8 using tissue from one animal donor.

Measurement of s-GAG Concentration

The sulfated glycosaminoglycan (s-GAG) was determined using the dimethylmethylene blue (DMMB) assay (Farndale R W, Buttle D J, Barrett A J, Improved quantitation and discrimination of sulphated glycosaminoglycans by use of dimethylmethylene blue. Biochim Biophys Acta 1986, 883, 173-177.) with a standard of shark cartilage chondroitin sulfate C (Sigma-Aldrich®, USA). The DMMB solution was used to dilute the sample, the standards and the appropriate blank solution. The absorbance of the resulting solution was measured at 525 nm using a microplate reader spectrophotometer.

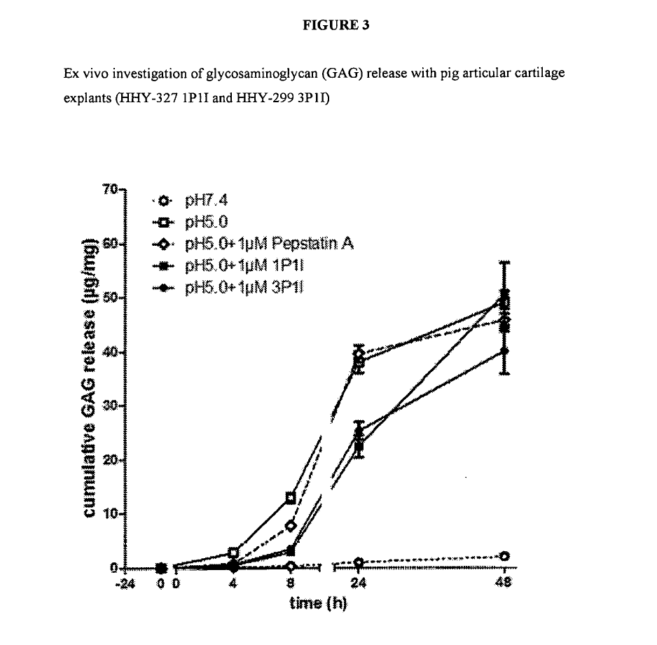

Results are shown in FIG. 3. It reflects GAG degradation in acidic (physiologically relevant) pH environment. It is observed that non-targeted native pepstatin A shows no effect at all, while conjugated, targeted drug compound HHY-327 (example 4, 1P1I in FIG. 3) displays a very impressive effect.

At 24 hours after stimulation, the negative control explants without pre-incubated cathepsin D inhibitor showed an approximately 40-fold increase in GAG release at pH 5.0 over controls (pH 7.4 buffer). The free pepstatin A pre-incubation was not effective at blocking aggrecan breakdown, represented by GAG release, which indicate that pepstatin A is not retained in the cartilage explants. In contrast, the acid-stimulated GAG release was significantly inhibited by the cartilage targeting conjugates. HHY-327 (1P1I in FIG. 3), carrying one targeting moiety, could inhibit 41% of GAG release at 24 hours but no differentiating effect could be observed at 48 hours after stimulation; while for the derivative with the inhibitor conjugated to three targeting peptides (HHY-299, example 7, 3P1I in FIG. 3), inhibition of 34% of GAG release at 24 hours and 18% at 48 hours was observed. The ex-vivo experiments in this very harsh model clearly demonstrate that the DOTAM-based inhibitor conjugate can deliver and retain a therapeutic molecule to cartilage tissue much more efficiently than this is possible for a non-conjugated inhibitor. Utilization of collagen targeting moiteies proved to be superior than purely charge-dependent effects. This is of particular importance, as GAG loss is pronounced even at early to moderate stages of OA, thereby resulting in an overall reduction of negative charges in cartilage layers. The active cartilage targeting properties and the resulting inhibitory effect can be prolonged by increasing the targeting peptide number from one to three on the adaptor.

Ex Vivo MRI Investigation with Pig Articular Cartilage Explants

1. Whole-depth pig articular cartilage (d=4 mm) was maintained in culture for 17 h to stabilize.

2. Incubated with MRI contrast agent (Gd-DTPA or MnCl₂) at 37° C. for 24 h. In achieve optimal penetration conditions, various concentrations (0.2 mM, 0.5 mM, 2 mM, 5 mM) were tested.

3. Measured with or without washing (3 times for 10 min each)

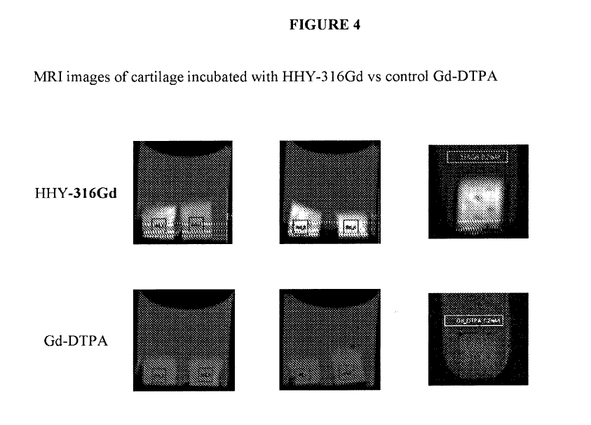

The T₁ signal enhancement of the targeting contrast agent HHY-316Gd in water was measured and compared with Gd-DTPA_ENREF_27, as commercially available contrast agent in clinical use (FIG. 4). The T₁ measurement for HHY-316Gd incubated cartilage showed a four-fold signal enhancement compared to Gd-DTPA (T1: 344 ms vs 1399 ms). The level of signal enhancement demonstrates the effectiveness of the cartilage targeting principle of HHY-316Gd for the visualization by MRI.

These ex-vivo cartilage experiments show the unambiguous superiority of the developed probe compared to commercial Gd-DTPA (DOTA based). Only the unique combination of charge effect (to target cartilage-contained GAG) and highly specific collagen II targeting peptide is responsible for this effect.

In Vivo MRI Investigation with Health Rats

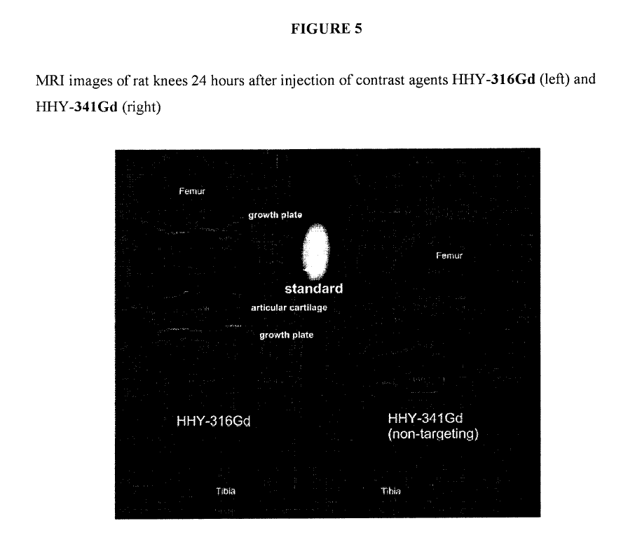

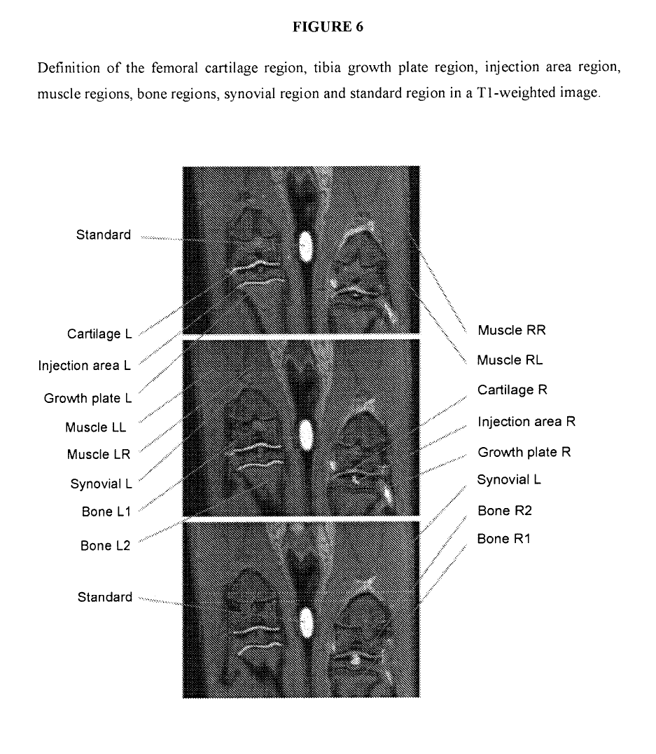

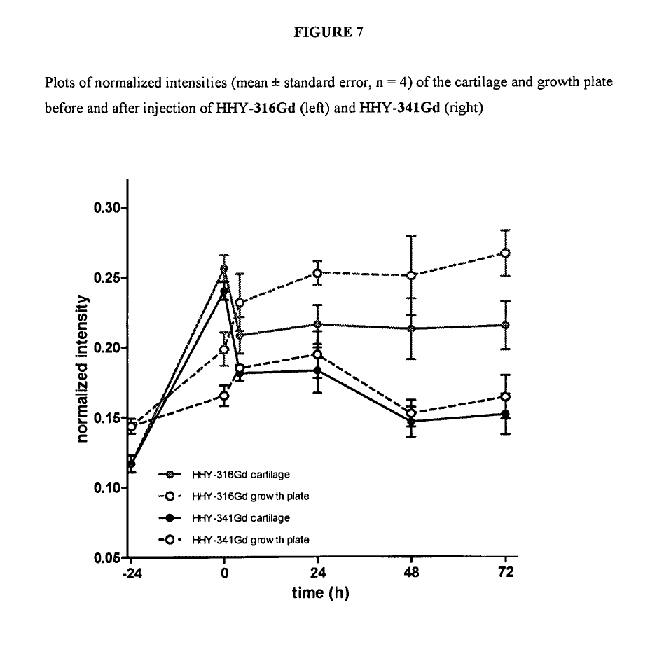

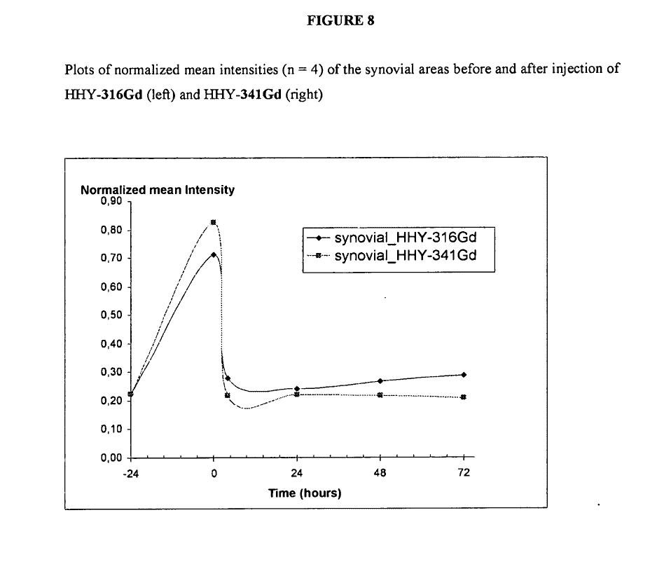

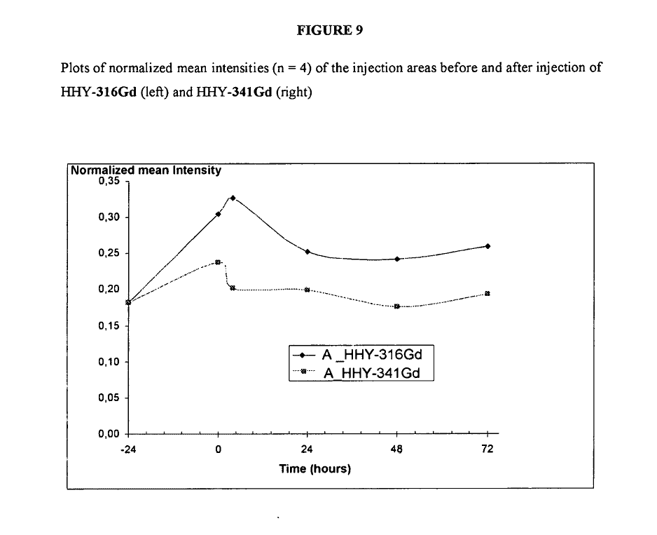

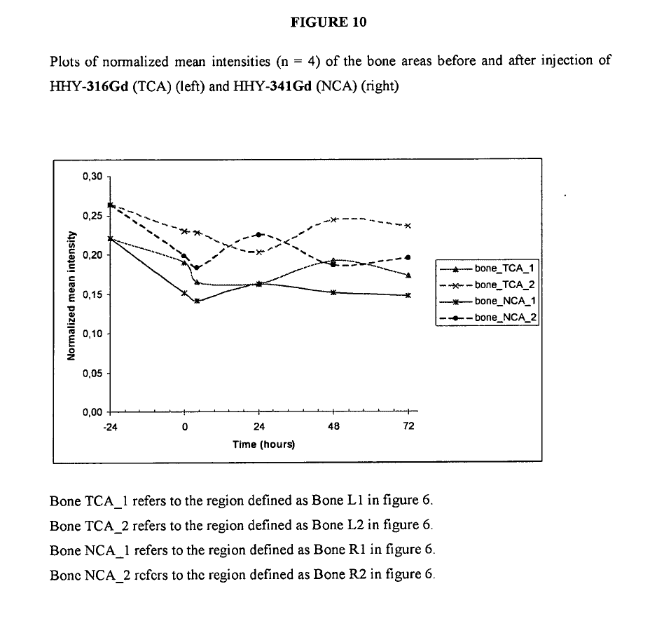

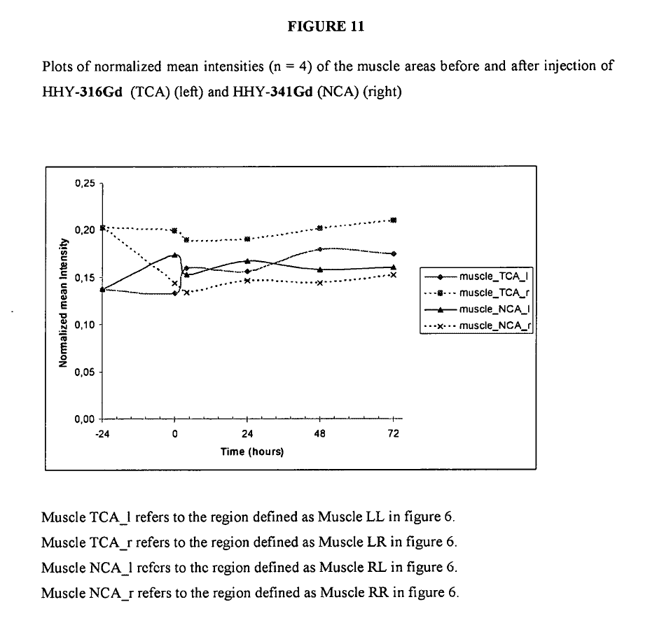

To explore the potential of cartilage targeting agents as practical MRI contrast agents for accurate diagnostic imaging, we investigated HHY-316Gd in vivo with healthy rats. As an essentially non-binding control, contrast agent HHY-341Gd (T₁=1368±10 ms, 0.2 mM in DPBS) with the non-binding scrambled peptide sequence YRLGRW and acetylated amine groups was used as reference. MRI was performed using a 4.7 T Bruker BioSpec whole body MR imager. Male skeletally mature Lewis rats were anesthetized (by Isoflurane), fixed in supine position and both knees were placed within a transmitting/receiving wrist coil. Both knees were visualized simultaneously allowing the comparison of one knee with the other, in which the labeling characteristics of two different contrast agents HHY-316Gd and HHY-341Gd and their clearance from the knee joint could be monitored over time Kinetic studies in rat knee joints showed a rapid clearance of both contrast agents from non-cartilage compartments of the joint like meniscus, synovium, fat tissues, muscles or bones within 4 hours. FIG. 5 shows the imaging obtained 24 hours after intra-articular injection of contrast agents. In the HHY-316Gd applied knees there is a significant MRI signal enhancement of the articular and growth plate cartilages which consist principally of collagen II and proteoglycan. The signal intensity (SI) in the articular cartilage and growth plate increased 86% and 77% after 24 hours following administration of HHY-316Gd and increased 84% and 85% at 72 hours after intra-articular injection, respectively (FIGS. 6-11). Comparison with the results obtained after intra-articular injection of the non-targeting contrast agent HHY-341Gd further confirm the assumption that the MRI signal enhancement of the articular and growth plate cartilage is generated by accumulation of HHY-316Gd due to the specific tissue targeting effect of the probe. In contrast, there is no SIs enhancement of the joint region. With HHY-341Gd before and 72 hours after intra-articular injection almost no accumulation of the non-targeting reference was observed

In Vivo MRI Investigation with ACLT-pMx Rats

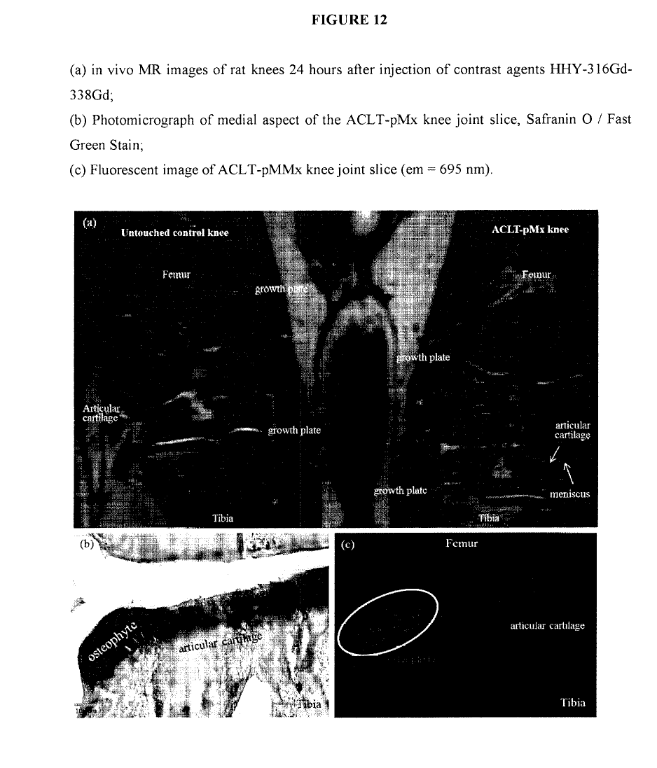

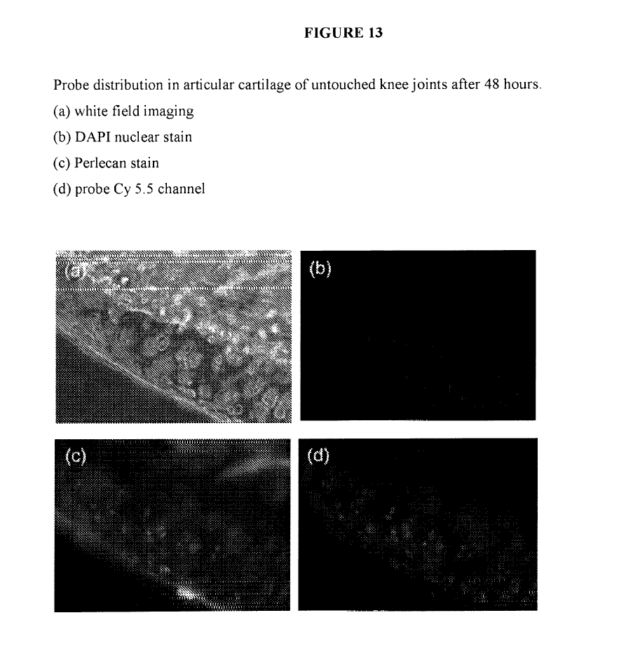

To assist histological examination of joint slices after termination of the study, we decided to co-administer a second probe, HHY-338 (T₁=11493±27 ms, 0.2 mM in DPBS), containing a Cy5.5 moiety. To account for the different sensitivities of optical and MRI based imaging, we chose to add only 1% of the fluorescent probe. 50 μL of a 10 mM DPBS solution of 1% of HHY-338 and 99% of HHY-316Gd was injected intra-articularly into both right knees with ACLT-pMx at day 28 after surgery and the left untouched control knees of rats (n=4). Post contrast images were taken immediately after administration and followed over 2 days post-administration (FIG. 12a). In the non-operated knee joints, a clear MRI signal enhancement of articular cartilage and growth plate compared to the operated knee could be observed. Interestingly, in the unstable ACLT-pMx knee, beside the increase of SIs of articular cartilage and growth plate, a region with brighter MRI signal was observed in the margin of medial tibial plateau indicating the formation of chondrophyte/osteophyte (red cycle). The results were further confirmed by histological studies. After 48 h post-administration, the rats were sacrificed, and the knee joints excised, frozen and sectioned for histology. Chondrophyte/osteophyte regions were examined in sections stained with safranin O/fast green and recorded using a Zeiss Miraxscan microscope (FIG. 12b). The non-operated joints exhibited no chondrophyte/osteophyte formation in the femoral condyles or tibial plateau. In contrast, the ACLT-pMx joints showed early chondrophyte/osteophyte (stage I) formation at the margin of medial tibial plateau. Fluorescence microscopy of the sectioned knee slices revealed a high level of fluorescence signal in the cartilage tissue, which is in agreement with the MRI data and shows the selective and high degree of accumulation of the probes in the cartilage tissue due to the intrinsic targeting effect. In the non-operated knee joint, the probe homogeneously distributed and entered articular cartilage, and NIRF signals were observed both in the matrix compartment and within chondrocytes. The pericellular matrix and the cell nuclei were substantially free from signal (FIG. 13). As shown in FIG. 12c, in the ACLT-pMx joint articular cartilage, besides the cartilage areas, more signals were obtained at the chondrophyte/osteophyte area which might be due to the expression of GAGs as well as collagen IIa, a splice variant of the collagen II gene expressed by chondroprogenitor cell, at early chondrophyte/osteophytes.

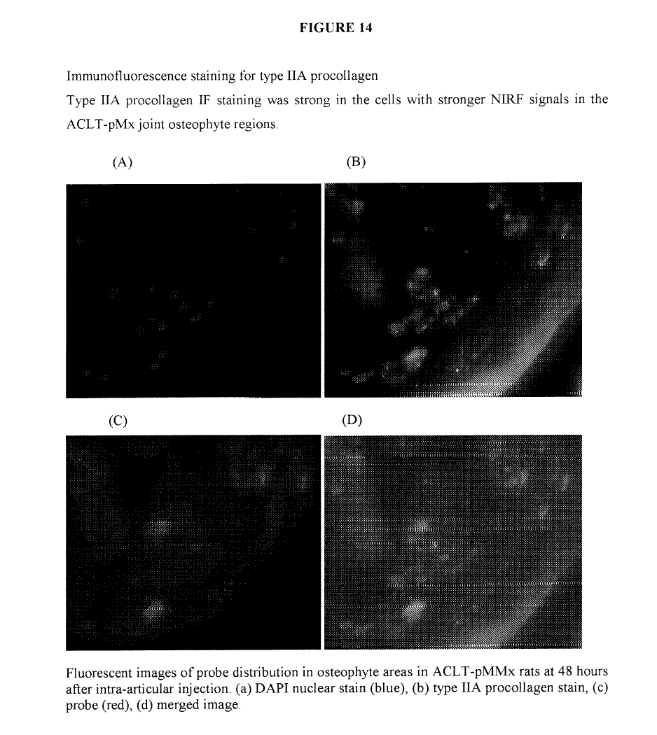

FIG. 14 shows the colocalisation of Type IIA procollagen and the probes are in the osteophyte regions.

1: A compound of formula (Ia), a stereoisomeric form thereof, or a pharmaceutically acceptable salt of any of the foregoing, 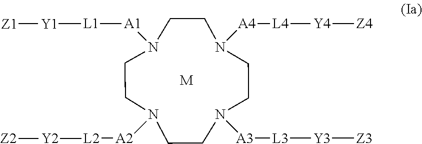
wherein
M is absent or present and, where present, is a positively charged metal ion selected from the group consisting of Gd, Yb, Mn, Cr, Cu, Fe, Pr, Nd, Sm, Tb, Yb, Dy, Ho, Er, Eu, Ga, ⁶⁸Ga, ⁶⁴Cu, ^{99m}Tc, ¹⁷⁷Lu, ⁶⁷Ga, ¹¹¹In, and ⁹⁹Mo;
A1, A2, A3 and A4 are independently selected from the group consisting of a bond, —(C₀-C₄)-alkyl-C(O)—N(R1)-, —(C₀-C₄)-alkyl-P(O)ₙ—N(R1)-, —(C₀-C₄)-alkyl-S(O)ₙ—N(R1)-, —(C₀-C₄)-alkyl-N(R2)-C(O)—N(R1)- and —(C₀-C₄)-alkyl-N(R1)-C(O)—;
n is 1 or 2;
L1, L2, L3 and L4 are independently selected from the group consisting of a bond, (C₁-C₁₈)-alkyl, and —(CH₂)ₘ[—O—(CH₂)ₚ]_{q}—;
m, q and p are independently the integer zero, 1, 2, 3, 4, 5, 6, 7, 8, 9 or 10;
Y1, Y2, Y3 and Y4 are independently selected from the group consisting of a bond, —(C₀-C₄)-alkyl-N(R1)-, —(C₀-C₄)-alkyl-C(O)—N(R1)-, —N(R1)-C(O)—(C₀-C₆)-alkyl, —(C₀-C₄)-alkyl-S(O)ₙ—N(R1)-, —(C₀-C₄)-alkyl-N(R2)-C(O)—N(R1)-, —(C₀-C₄)-alkyl-N(R1)-C(O)—, 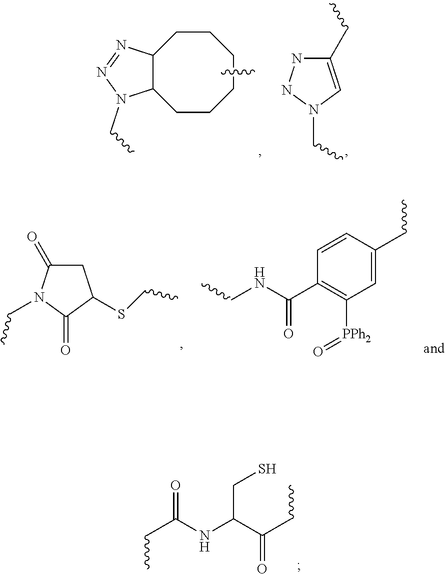
R1 and R2 are independently selected from the group consisting of hydrogen, (C₁-C₄)-alkyl, (C₃-C₇)-cycloalkyl and —(C₁-C₄)-alkyl-(C₃-C₇)-cycloalkyl; and
Z1, Z2, Z3 and Z4 are independently selected from the group consisting of:
a hydrogen atom,
a polypeptide comprising an amino acid sequence of WYRGRL-(SEQ ID NO:1), or a conservative substitution or a repetitive sequence thereof, wherein the polypeptide specifically binds to cartilage tissue, and wherein the N-terminal moiety of the polypeptide is acetylated,
a fluorophore suitable for optical imaging, and
an activity based probe (ABP) suitable to monitor aberrant expression or activity of proteins involved in the initiation and progression of osteoarthritis (OA);
provided that:
at least one of Z1, Z2, Z3 and Z4 is a polypeptide comprising an amino acid sequence of WYRGRL-(SEQ ID NO:1), or a conservative substitution or a repetitive sequence thereof, wherein the polypeptide specifically binds to cartilage tissue, and wherein the N-terminal moiety of the polypeptide is acetylated,
at least one of Z1, Z2, Z3 and Z4 is a fluorophore suitable for optical imaging or an activity based probe (ABP) suitable to monitor aberrant expression or activity of proteins involved in the initiation and progression of OA, and
not more than one of Z1, Z2, Z3 and Z4 is an activity based probe (ABP) suitable to monitor aberrant expression or activity of proteins involved in the initiation and progression of OA.

2: A compound of formula (Ib), a stereoisomeric form thereof, or a pharmaceutically acceptable salt of any of the foregoing, 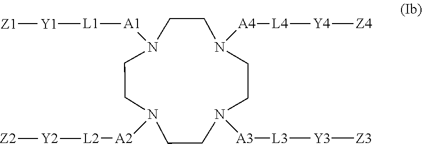
wherein
A1, A2, A3 and A4 are independently selected from the group consisting of a bond, —(C₀-C₄)-alkyl-C(O)—N(R1)-, —(C₀-C₄)-alkyl-P(O)ₙ—N(R1)-, —(C₀-C₄)-alkyl-S(O)ₙ—N(R1)-, —(C₀-C₄)-alkyl-N(R2)-C(O)—N(R1)- and —(C₀-C₄)-alkyl-N(R1)-C(O)—;
n is 1 or 2;
L1, L2, L3 and L4 are independently selected from the group consisting of a bond, (C₁-C₁₈)-alkyl, and —(CH₂)ₘ[—O—(CH₂)ₚ]_{q}—;
m, q and p are independently the integer zero, 1, 2, 3, 4, 5, 6, 7, 8, 9 or 10;
Y1, Y2, Y3 and Y4 are independently selected from the group consisting of a bond, a cleavable linker, —Y5-(Z5)ᵣ, —(C₀-C₄)-alkyl-N(R1)-, —(C₀-C₄)-alkyl-C(O)—N(R1)-, —N(R1)-C(O)—(C₀-C₆)-alkyl-, —(C₀-C₄)-alkyl-S(O)ₙ—N(R1)-, —(C₀-C₄)-alkyl-N(R2)-C(O)—N(R1)-, —(C₀-C₄)-alkyl-N(R1)-C(O)—, 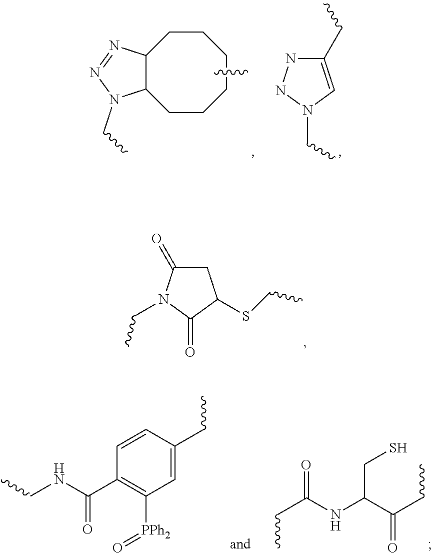
r is an integer from 1 to 3;
Y5 is selected from the group consisting of a cleavable linker, (C₀-C₄)-alkyl-N(R1), (C₀-C₄)-alkyl-C(O)—N(R1), N(R1)-C(O)—(C₀-C₆)-alkyl, (C₀-C₄)-alkyl-S(O)ₙ—N(R1), (C₀-C₄)-alkyl-N(R2)-C(O)—N(R1), and (C₀-C₄)-alkyl-N(R1)-C(O);
R1 and R2 are independently selected from the group consisting of hydrogen, (C₁-C₄)-alkyl, (C₃-C₇)-cycloalkyl and —(C₁-C₄)-alkyl-(C₃-C₇)-cycloalkyl; and
Z1, Z2, Z3, Z4, and Z5 are independently selected from the group consisting of:
a hydrogen atom,
a polypeptide comprising an amino acid sequence of WYRGRL-(SEQ ID NO:1), or a conservative substitution or a repetitive sequence thereof, wherein the polypeptide specifically binds to cartilage tissue, and wherein the N-terminal moiety of the polypeptide is acetylated, and
a compound useful for treatment of osteoarthritis, complications related to osteoarthritis or cartilage related disorders;
provided that:
at least one of Z1, Z2, Z3 and Z4 is a polypeptide comprising an amino acid sequence of WYRGRL-(SEQ ID NO:1), or a conservative substitution or a repetitive sequence thereof, wherein the polypeptide specifically binds to cartilage tissue, and wherein the N-terminal moiety of the polypeptide is acetylated, and
at least one of Z1, Z2, Z3 and Z4 is a compound that is useful for treatment of osteoarthritis, complications related to osteoarthritis or cartilage related disorders.

3: A compound of formula (Ia), a stereoisomeric form thereof, or a pharmaceutically acceptable salt of any of the foregoing, 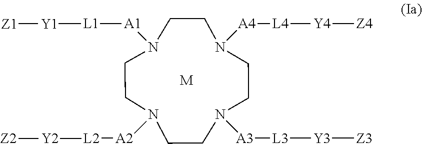
wherein
M is absent or present and, where present, is a positively charged metal ion selected from the group consisting of Gd, Yb, Mn, Cr, Cu, Fe, Pr, Nd, Sm, Tb, Yb, Dy, Ho, Er, Eu, Ga, ⁶⁸Ga, ⁶⁴Cu, ^{99m}Tc, ¹⁷⁷Lu, ⁶⁷Ga, ¹¹¹In, and ⁹⁹Mo;
A1, A2, A3 and A4 are independently selected from the group consisting of a bond, —(C₀-C₄)-alkyl-C(O)—N(R1)-, —(C₀-C₄)-alkyl-P(O)ₙ—N(R1)-, —(C₀-C₄)-alkyl-S(O)ₙ—N(R1)-, —(C₀-C₄)-alkyl-N(R2)-C(O)—N(R1)- and —(C₀-C₄)-alkyl-N(R1)-C(O)—;
n is 1 or 2;
L1, L2, L3 and L4 are independently selected from the group consisting of a bond, (C₁-C₁₈)-alkyl, and —(CH₂)ₘ[—O—(CH₂)ₚ]_{q}—;
m, q and p are independently the integer zero, 1, 2, 3, 4, 5, 6, 7, 8, 9 or 10;
Y1, Y2, Y3 and Y4 are independently selected from the group consisting of a bond, —(C₀-C₄)-alkyl-N(R1)-, —(C₀-C₄)-alkyl-C(O)—N(R1)-, —N(R1)-C(O)—(C₀-C₆)-alkyl-, —(C₀-C₄)-alkyl-S(O)ₙ—N(R1)-, —(C₀-C₄)-alkyl-N(R2)-C(O)—N(R1)-, —(C₀-C₄)-alkyl-N(R1)-C(O)—, 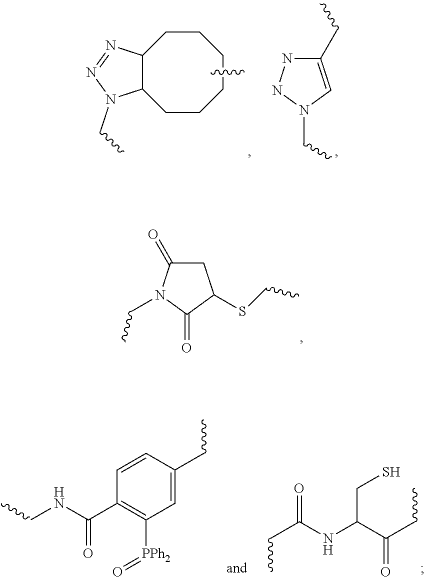
R1 and R2 are independently selected from the group consisting of hydrogen, (C₁-C₄)-alkyl, (C₃-C₇)-cycloalkyl and —(C₁-C₄)-alkyl-(C₃-C₇)-cycloalkyl; and
Z1, Z2, Z3 and Z4 are independently selected from the group consisting of:
a hydrogen atom,
a polypeptide comprising an amino acid sequence of WYRGRL-(SEQ ID NO:1), or a conservative substitution or a repetitive sequence thereof, wherein the polypeptide specifically binds to cartilage tissue, and wherein the N-terminal moiety of the polypeptide is acetylated,
a fluorophore, and
an activity based probe (ABP);
provided that:
at least one of Z1, Z2, Z3 and Z4 is a polypeptide comprising an amino acid sequence of WYRGRL-(SEQ ID NO:1), or a conservative substitution or a repetitive sequence thereof, wherein the polypeptide specifically binds to cartilage tissue, and wherein the N-terminal moiety of the polypeptide is acetylated,
at least one of Z1, Z2, Z3 and Z4 is a fluorophore or an activity based probe (ABP), and
not more than one of Z1, Z2, Z3 and Z4 is an activity based probe (ABP).

4: The compound of claim 2, a stereoisomeric form thereof, or a pharmaceutically acceptable salt of any of the foregoing, wherein the compound useful for treatment of osteoarthritis, complications related to osteoarthritis or cartilage related disorders is an inhibitor of MMP13; recombinant metalloproteinase inhibitor-3; ADAMTS4/5; Cathepsin D; a Cathepsin K inhibitor; an inhibitor of Cathepsin S or B; a serine protease inhibitor against uPA, tPA, HTRA1, or PACE4; an inhibitor of the complement system; an inhibitor against C1s, C1r, C3, CS or Membrane attack complex; an inhibitor against proinflammtory cytokines; interleukin 1 converting enzyme; recombinant Interleukin 1 receptor antagonist; an inhibitor against proinflammatory intracellular signaling pathways or p38-pathways; an inhibitor of FAK-signaling; an inhibitor against Toll-like receptors; doxycycline; glucosamin-hydrochloride; chondroitin sulfate; an inhibitor of canonical WNT signaling; an inhibitor of Frizzled receptors; a modulator of GSK3β; an inhibitor of SGK-1; recombinant Wif1; recombinant SFRP; recombinant DKK-1; an inhibitor of LRP5 or LRP6; recombinant Sost; an inhibitor of chondrocyte hypertrophic differentiation; an HDAC-4 modulator; an FGFR3 agonist; recombinant PTHrP; a chondrocyte anabolic molecule; an activator of TGFβ-Smad2-/3 signaling; an activator of BMP-/Samd1,-5-,-8 signaling; a modulator of Notch signaling; a tyrpsone kinase inhibitor; a Syndecan-4 inhibitor; an inhibitor of leptin or leptin signaling; a therapeutic principle with primary symptomatic treatment; an inhibitor of TRPV1; an inhibitor of P2X7; an inhibitor of Nav1.7; an inhibitor of PGE2-formation; a non-steroidal anti-inflammatory drug; a corticosteroid; an inhibitor of TrkA; or a COX2 inhibitor.

5: A method of diagnosing or monitoring aberrant expression or activity of proteins involved in the initiation and progression of OA in an animal in need thereof comprising administering the compound of claim 1, a stereoisomeric form thereof or a mixture of stereoisomeric forms thereof in any ratio, or a pharmaceutically acceptable salt of any of the foregoing, to said animal.

6: A method of treating OA in an animal in need thereof comprising administering the compound of claim 1, a stereoisomeric form thereof or a mixture of stereoisomeric forms thereof in any ratio, or a pharmaceutically acceptable salt of any of the foregoing, to said animal.

7: A method of treating OA in an animal in need thereof comprising administering the compound of claim 2, a stereoisomeric form thereof or a mixture of stereoisomeric forms thereof in any ratio, or a pharmaceutically acceptable salt of any of the foregoing, to said animal.

8: A method of preventing OA in an animal in need thereof comprising administering the compound of claim 1, a stereoisomeric form thereof or a mixture of stereoisomeric forms thereof in any ratio, or a pharmaceutically acceptable salt of any of the foregoing, to said animal.

9: A method of treating pain associated with OA in an animal in need thereof comprising administering the compound of claim 1, a stereoisomeric form thereof or a mixture of stereoisomeric forms thereof in any ratio, or a pharmaceutically acceptable salt of any of the foregoing, to said animal.

10: A pharmaceutical composition comprising the compound of claim 1, a stereoisomeric form thereof or a mixture of stereoisomeric forms thereof in any ratio, or a pharmaceutically acceptable salt of any of the foregoing, and a pharmaceutically acceptable carrier.

11: A pharmaceutical composition comprising the compound of claim 2, a stereoisomeric form thereof or a mixture of stereoisomeric forms thereof in any ratio, or a pharmaceutically acceptable salt of any of the foregoing, and a pharmaceutically acceptable carrier.

12: A method of preventing OA in an animal in need thereof comprising administering the compound of claim 2, a stereoisomeric form thereof or a mixture of stereoisomeric forms thereof in any ratio, or a pharmaceutically acceptable salt of any of the foregoing, to said animal.

13: A method of preventing OA in an animal in need thereof comprising administering the compound of claim 3, a stereoisomeric form thereof or a mixture of stereoisomeric forms thereof in any ratio, or a pharmaceutically acceptable salt of any of the foregoing, to said animal.

14: A method of treating OA in an animal in need thereof comprising administering the compound of claim 3, a stereoisomeric form thereof or a mixture of stereoisomeric forms thereof in any ratio, or a pharmaceutically acceptable salt of any of the foregoing, to said animal.

15: A method of treating pain associated with OA in an animal in need thereof comprising administering the compound of claim 2, a stereoisomeric form thereof or a mixture of stereoisomeric forms thereof in any ratio, or a pharmaceutically acceptable salt of any of the foregoing, to said animal.

16: A method of treating pain associated with OA in an animal in need thereof comprising administering the compound of claim 3, a stereoisomeric form thereof or a mixture of stereoisomeric forms thereof in any ratio, or a pharmaceutically acceptable salt of any of the foregoing, to said animal.

17: A pharmaceutical composition comprising the compound of claim 3, a stereoisomeric form thereof or a mixture of stereoisomeric forms thereof in any ratio, or a pharmaceutically acceptable salt of any of the foregoing, and a pharmaceutically acceptable carrier.
